SEQUENCE LISTING (92 sequences)

SEQ ID NO: 1 - protein, 247 aa, Artificial Sequence
  Synthetic Construct
  MISC_FEATURE (156)..(156): Xaa is Asp or Glu
  MISC_FEATURE (158)..(158): Xaa is Leu or Met
NVNHKPSNTKVDKKVEPKSSDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRXEXTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 247 aa, Artificial Sequence
  Synthetic Construct
NVNHKPSNTKVDKKVEPKSSDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 247 aa, Artificial Sequence
  Synthetic Construct
NVNHKPSNTKVDKKVEPKSSDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 247 aa, Artificial Sequence
  Synthetic Construct
  MISC_FEATURE (156)..(156): Xaa is Asp or Glu
  MISC_FEATURE (158)..(158): Xaa is Leu or Met
NVNHKPSNTKVDKKVEPKSSDKTHTCPPCPAPELLGGPSVFLFPPKPKDTLYITREPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRXEXTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 247 aa, Artificial Sequence
  Synthetic Construct
NVNHKPSNTKVDKKVEPKSSDKTHTCPPCPAPELLGGPSVFLFPPKPKDTLYITREPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 247 aa, Artificial Sequence
  Synthetic Construct
NVNHKPSNTKVDKKVEPKSSDKTHTCPPCPAPELLGGPSVFLFPPKPKDTLYITREPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - protein, 247 aa, Artificial Sequence
  Synthetic Construct
  MISC_FEATURE (1)..(1): Xaa is Asn or absent
  MISC_FEATURE (97)..(97): Xaa is Asn or Ala
  MISC_FEATURE (156)..(156): Xaa is Asp or Glu
  MISC_FEATURE (158)..(158): Xaa is Leu or Met
  MISC_FEATURE (247)..(247): Xaa is Lys or absent
XVNHKPSNTKVDKKVEPKSSDKTHTCPPCPAPELLGGPSVFLFPPKPKDTLYITREPEVTCVVVDVSHEDPEVKFNWYVDGVEVHNAKTKPREEQYXSTYRVVSVLTVLHQDWLNGKEYKCKVSNKALPAPIEKTISKAKGQPREPQVYTLPPSRXEXTKNQVSLTCLVKGFYPSDIAVEWESNGQPENNYKTTPPVLDSDGSFFLYSKLTVDKSRWQQGNVFSCSVMHEALHNHYTQKSLSLSPGX

SEQ ID NO: 8 - protein, 246 aa, Artificial Sequence
  Synthetic Construct
VNHKPSNTKVDKKVEPKSSDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - protein, 246 aa, Artificial Sequence
  Synthetic Construct
VNHKPSNTKVDKKVEPKSSDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 246 aa, Artificial Sequence
  Synthetic Construct
NVNHKPSNTKVDKKVEPKSSDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 11 - protein, 246 aa, Artificial Sequence
  Synthetic Construct
NVNHKPSNTKVDKKVEPKSSDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 12 - protein, 245 aa, Artificial Sequence
  Synthetic Construct
VNHKPSNTKVDKKVEPKSSDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 13 - protein, 245 aa, Artificial Sequence
  Synthetic Construct
VNHKPSNTKVDKKVEPKSSDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 14 - protein, 246 aa, Artificial Sequence
  Synthetic Construct
VNHKPSNTKVDKKVEPKSSDKTHTCPPCPAPELLGGPSVFLFPPKPKDTLYITREPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 15 - protein, 246 aa, Artificial Sequence
  Synthetic Construct
VNHKPSNTKVDKKVEPKSSDKTHTCPPCPAPELLGGPSVFLFPPKPKDTLYITREPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - protein, 246 aa, Artificial Sequence
  Synthetic Construct\
NVNHKPSNTKVDKKVEPKSSDKTHTCPPCPAPELLGGPSVFLFPPKPKDTLYITREPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 17 - protein, 246 aa, Artificial Sequence
  Synthetic Construct
NVNHKPSNTKVDKKVEPKSSDKTHTCPPCPAPELLGGPSVFLFPPKPKDTLYITREPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 18 - protein, 245 aa, Artificial Sequence
  Synthetic Construct
VNHKPSNTKVDKKVEPKSSDKTHTCPPCPAPELLGGPSVFLFPPKPKDTLYITREPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 19 - protein, 245 aa, Artificial Sequence
  Synthetic Construct
VNHKPSNTKVDKKVEPKSSDKTHTCPPCPAPELLGGPSVFLFPPKPKDTLYITREPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 20 - protein, 247 aa, Artificial Sequence
  Synthetic Construct
  MISC_FEATURE (1)..(1): Xaa is Asn or absent
  MISC_FEATURE (156)..(156): Xaa is Asp or Glu
  MISC_FEATURE (158)..(158): Xaa is Leu or Met
  MISC_FEATURE (247)..(247): Xaa is Lys is absent
XVNHKPSNTKVDKKVEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRXEXTKNQVSLTCLVKGFYPSDIAVEWESNGQPENNYKTTPPVLDSDGSFFLYSKLTVDKSRWQQGNVFSCSVMHEALHNHYTQKSLSLSPGX

SEQ ID NO: 21 - protein, 247 aa, Artificial Sequence
  Synthetic Construct
NVNHKPSNTKVDKKVEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 22 - protein, 247 aa, Artificial Sequence
  Synthetic Construct
NVNHKPSNTKVDKKVEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 23 - protein, 247 aa, Artificial Sequence
  Synthetic Construct
  MISC_FEATURE (1)..(1): Xaa is Asn or absent
  MISC_FEATURE (52)..(52): Xaa is Met or Tyr
  MISC_FEATURE (54)..(54): Xaa is Ser or Thr
  MISC_FEATURE (56)..(56): Xaa is Thr or Glu
  misc_feature (97)..(97): Xaa can be any naturally occurring amino acid
  MISC_FEATURE (156)..(156): Xaa is Asp or Glu
  MISC_FEATURE (158)..(158): Xaa is Leu or Met
  MISC_FEATURE (247)..(247): Xaa is Lys or absent
XVNHKPSNTKVDKKVEPKSSDKTHTCPPCPAPELLGGPSVFLFPPKPKDTLXIXRXPEVTCVVVDVSHEDPEVKFNWYVDGVEVHNAKTKPREEQYXSTYRVVSVLTVLHQDWLNGKEYKCKVSNKALPAPIEKTISKAKGQPREPQVYTLPPSRXEXTKNQVSLTCLVKGFYPSDIAVEWESNGQPENNYKTTPPVLDSDGSFFLYSKLTVDKSRWQQGNVFSCSVMHEALHNHYTQKSLSLSPGX

SEQ ID NO: 24 - protein, 246 aa, Artificial Sequence
  Synthetic Construct
  MISC_FEATURE (51)..(51): Xaa is Met or Tyr
  MISC_FEATURE (53)..(53): Xaa is Ser or Thr
  MISC_FEATURE (55)..(55): Xaa is Thr or Glu
  MISC_FEATURE (96)..(96): Xaa is Asn or Ala
  MISC_FEATURE (155)..(155): Xaa is Asp or Glu
  MISC_FEATURE (157)..(157): Xaa is Leu or Met
VNHKPSNTKVDKKVEPKSSDKTHTCPPCPAPELLGGPSVFLFPPKPKDTLXIXRXPEVTCVVVDVSHEDPEVKFNWYVDGVEVHNAKTKPREEQYXSTYRVVSVLTVLHQDWLNGKEYKCKVSNKALPAPIEKTISKAKGQPREPQVYTLPPSRXEXTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 25 - protein, 246 aa, Artificial Sequence
  Synthetic Construct
  MISC_FEATURE (51)..(51): Xaa is Met or Tyr
  MISC_FEATURE (53)..(53): Xaa is Ser or Thr
  MISC_FEATURE (55)..(55): Xaa is Thr or Glu
  MISC_FEATURE (96)..(96): Xaa is Asn or Ala
VNHKPSNTKVDKKVEPKSSDKTHTCPPCPAPELLGGPSVFLFPPKPKDTLXIXRXPEVTCVVVDVSHEDPEVKFNWYVDGVEVHNAKTKPREEQYXSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 26 - protein, 246 aa, Artificial Sequence
  Synthetic Construct
  MISC_FEATURE (51)..(51): Xaa is Met or Tyr
  MISC_FEATURE (53)..(53): Xaa is Ser or Thr
  MISC_FEATURE (55)..(55): Xaa is Thr or Glu
  MISC_FEATURE (96)..(96): Xaa is Asn or Ala
VNHKPSNTKVDKKVEPKSSDKTHTCPPCPAPELLGGPSVFLFPPKPKDTLXIXRXPEVTCVVVDVSHEDPEVKFNWYVDGVEVHNAKTKPREEQYXSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 27 - protein, 245 aa, Artificial Sequence
  Synthetic Construct
  MISC_FEATURE (51)..(51): Xaa is Met or Tyr
  MISC_FEATURE (53)..(53): Xaa is Ser or Thr
  MISC_FEATURE (55)..(55): Xaa is Thr or Glu
  MISC_FEATURE (96)..(96): Xaa is Asn or Ala
  MISC_FEATURE (155)..(155): Xaa is Asp or Glu
  MISC_FEATURE (157)..(157): Xaa is Leu or Met
VNHKPSNTKVDKKVEPKSSDKTHTCPPCPAPELLGGPSVFLFPPKPKDTLXIXRXPEVTCVVVDVSHEDPEVKFNWYVDGVEVHNAKTKPREEQYXSTYRVVSVLTVLHQDWLNGKEYKCKVSNKALPAPIEKTISKAKGQPREPQVYTLPPSRXEXTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 28 - protein, 245 aa, Artificial Sequence
  Synthetic Construct
  MISC_FEATURE (51)..(51): Xaa is Met or Tyr
  MISC_FEATURE (53)..(53): Xaa is Ser or Thr
  MISC_FEATURE (55)..(55): Xaa is Thr or Glu
  MISC_FEATURE (96)..(96): Xaa is Asn or Ala
VNHKPSNTKVDKKVEPKSSDKTHTCPPCPAPELLGGPSVFLFPPKPKDTLXIXRXPEVTCVVVDVSHEDPEVKFNWYVDGVEVHNAKTKPREEQYXSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 29 - protein, 245 aa, Artificial Sequence
  Synthetic Construct
  MISC_FEATURE (51)..(51): Xaa is Met or Tyr
  MISC_FEATURE (53)..(53): Xaa is Ser or Thr
  MISC_FEATURE (55)..(55): Xaa is Thr or Glu
  MISC_FEATURE (96)..(96): Xaa is Asn or Ala
VNHKPSNTKVDKKVEPKSSDKTHTCPPCPAPELLGGPSVFLFPPKPKDTLXIXRXPEVTCVVVDVSHEDPEVKFNWYVDGVEVHNAKTKPREEQYXSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 30 - protein, 266 aa, Artificial Sequence
  Synthetic Construct
MGWSCIILFLVATATGVHSNVNHKPSNTKVDKKVEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 31 - protein, 247 aa, Artificial Sequence
  Synthetic Construct
  MISC_FEATURE (1)..(1): Xaa is Asn or absent
  MISC_FEATURE (20)..(20): Xaa is Cys or Ser
  MISC_FEATURE (52)..(52): Xaa is Met or Tyr
  MISC_FEATURE (54)..(54): Xaa is Ser or Thr
  MISC_FEATURE (56)..(56): Xaa is Thr or Glu
  MISC_FEATURE (97)..(97): Xaa is Asn or Ala
  MISC_FEATURE (109)..(109): Xaa is Leu or Asp
  MISC_FEATURE (111)..(111): Xaa is Gln or His
  MISC_FEATURE (156)..(156): Xaa is Asp or Glu
  MISC_FEATURE (158)..(158): Xaa is Leu or Met
  MISC_FEATURE (228)..(228): Xaa is Asn or Ser
  MISC_FEATURE (234)..(234): Xaa is Asn or Ser
  MISC_FEATURE (247)..(247): Xaa is Lys or Absent
XVNHKPSNTKVDKKVEPKSXDKTHTCPPCPAPELLGGPSVFLFPPKPKDTLXIXRXPEVTCVVVDVSHEDPEVKFNWYVDGVEVHNAKTKPREEQYXSTYRVVSVLTVXHXDWLNGKEYKCKVSNKALPAPIEKTISKAKGQPREPQVYTLPPSRXEXTKNQVSLTCLVKGFYPSDIAVEWESNGQPENNYKTTPPVLDSDGSFFLYSKLTVDKSRWQQGNVFSCSVXHEALHXHYTQKSLSLSPGX

SEQ ID NO: 32 - protein, 247 aa, Artificial Sequence
  Synthetic Construct
  MISC_FEATURE (1)..(1): Xaa is Asn or absent
  MISC_FEATURE (97)..(97): Xaa is Asn or Ala
  MISC_FEATURE (109)..(109): Xaa is Leu or Asp
  MISC_FEATURE (111)..(111): Xaa is Gln or His
  MISC_FEATURE (156)..(158): Xaa is Asp or Glu
  MISC_FEATURE (158)..(158): Xaa is Leu or Met
  MISC_FEATURE (228)..(228): Xaa is Met or Leu
  MISC_FEATURE (234)..(234): Xaa is Asn or Ser
  MISC_FEATURE (247)..(247): Xaa is Lys or absent
XVNHKPSNTKVDKKVEPKSSDKTHTCPPCPAPELLGGPSVFLFPPKPKDTLMISRTPEVTCVVVDVSHEDPEVKFNWYVDGVEVHNAKTKPREEQYXSTYRVVSVLTVXHXDWLNGKEYKCKVSNKALPAPIEKTISKAKGQPREPQVYTLPPSRXEXTKNQVSLTCLVKGFYPSDIAVEWESNGQPENNYKTTPPVLDSDGSFFLYSKLTVDKSRWQQGNVFSCSVXHEALHXHYTQKSLSLSPGX

SEQ ID NO: 33 - protein, 247 aa, Artificial Sequence
  Synthetic Construct
  MISC_FEATURE (1)..(1): Xaa is Asn or absent
  MISC_FEATURE (97)..(97): Xaa is Asn or Ala
  MISC_FEATURE (156)..(156): Xaa is Asp or Glu
  MISC_FEATURE (158)..(158): Xaa is Leu or Met
  MISC_FEATURE (228)..(228): Xaa is Met or Leu
  MISC_FEATURE (234)..(234): Xaa is Asn or Ser
  MISC_FEATURE (247)..(247): Xaa is Lys or absent
XVNHKPSNTKVDKKVEPKSSDKTHTCPPCPAPELLGGPSVFLFPPKPKDTLMISRTPEVTCVVVDVSHEDPEVKFNWYVDGVEVHNAKTKPREEQYXSTYRVVSVLTVDHHDWLNGKEYKCKVSNKALPAPIEKTISKAKGQPREPQVYTLPPSRXEXTKNQVSLTCLVKGFYPSDIAVEWESNGQPENNYKTTPPVLDSDGSFFLYSKLTVDKSRWQQGNVFSCSVXHEALHXHYTQKSLSLSPGX

SEQ ID NO: 34 - protein, 247 aa, Artificial Sequence
  Synthetic Construct
  MISC_FEATURE (97)..(97): Xaa is Asn or Ala
  MISC_FEATURE (156)..(156): Xaa is Asp or Glu
  MISC_FEATURE (158)..(158): Xaa is Leu or Met
NVNHKPSNTKVDKKVEPKSSDKTHTCPPCPAPELLGGPSVFLFPPKPKDTLMISRTPEVTCVVVDVSHEDPEVKFNWYVDGVEVHNAKTKPREEQYXSTYRVVSVLTVDHHDWLNGKEYKCKVSNKALPAPIEKTISKAKGQPREPQVYTLPPSRXEXTKNQVSLTCLVKGFYPSDIAVEWESNGQPENNYKTTPPVLDSDGSFFLYSKLTVDKSRWQQGNVFSCSVMHEALHSHYTQKSLSLSPGK

SEQ ID NO: 35 - protein, 247 aa, Artificial Sequence
  Synthetic Construct
  MISC_FEATURE (156)..(156): Xaa is Asp or Glu
  MISC_FEATURE (158)..(158): Xaa is Leu or Met
NVNHKPSNTKVDKKVEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVDHHDWLNGKEYKCKVSNKALPAPIEKTISKAKGQPREPQVYTLPPSRXEXTKNQVSLTCLVKGFYPSDIAVEWESNGQPENNYKTTPPVLDSDGSFFLYSKLTVDKSRWQQGNVFSCSVMHEALHSHYTQKSLSLSPGK

SEQ ID NO: 36 - protein, 247 aa, Artificial Sequence
  Synthetic Construct
NVNHKPSNTKVDKKVEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVDHHDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHSHYTQKSLSLSPGK

SEQ ID NO: 37 - protein, 247 aa, Artificial Sequence
  Synthetic Construct
NVNHKPSNTKVDKKVEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVDHHDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHSHYTQKSLSLSPGK

SEQ ID NO: 38 - protein, 246 aa, Artificial Sequence
  Synthetic Construct
VNHKPSNTKVDKKVEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVDHHDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHSHYTQKSLSLSPGK

SEQ ID NO: 39 - protein, 246 aa, Artificial Sequence
  Synthetic Construct
VNHKPSNTKVDKKVEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVDHHDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHSHYTQKSLSLSPGK

SEQ ID NO: 40 - protein, 247 aa, Artificial Sequence; identical to SEQ ID NO: 36
  Synthetic Construct

SEQ ID NO: 41 - protein, 247 aa, Artificial Sequence; identical to SEQ ID NO: 37
  Synthetic Construct

SEQ ID NO: 42 - protein, 245 aa, Artificial Sequence
  Synthetic Construct
VNHKPSNTKVDKKVEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVDHHDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHSHYTQKSLSLSPG

SEQ ID NO: 43 - protein, 245 aa, Artificial Sequence
  Synthetic Construct
VNHKPSNTKVDKKVEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVDHHDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHSHYTQKSLSLSPG

SEQ ID NO: 44 - protein, 247 aa, Artificial Sequence
  Synthetic Construct
  MISC_FEATURE (156)..(156): Xaa is Asp or Glu
  MISC_FEATURE (158)..(158): Xaa is Leu or Met
NVNHKPSNTKVDKKVEPKSSDKTHTCPPCPAPELLGGPSVFLFPPKPKDTLMISRTPEVTCVVVDVSHEDPEVKFNWYVDGVEVHNAKTKPREEQYASTYRVVSVLTVDHHDWLNGKEYKCKVSNKALPAPIEKTISKAKGQPREPQVYTLPPSRXEXTKNQVSLTCLVKGFYPSDIAVEWESNGQPENNYKTTPPVLDSDGSFFLYSKLTVDKSRWQQGNVFSCSVMHEALHSHYTQKSLSLSPGK

SEQ ID NO: 45 - protein, 247 aa, Artificial Sequence
  Synthetic Construct
NVNHKPSNTKVDKKVEPKSSDKTHTCPPCPAPELLGGPSVFLFPPKPKDTLMISRTPEVTCVVVDVSHEDPEVKFNWYVDGVEVHNAKTKPREEQYASTYRVVSVLTVDHHDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHSHYTQKSLSLSPGK

SEQ ID NO: 46 - protein, 247 aa, Artificial Sequence
  Synthetic Construct
NVNHKPSNTKVDKKVEPKSSDKTHTCPPCPAPELLGGPSVFLFPPKPKDTLMISRTPEVTCVVVDVSHEDPEVKFNWYVDGVEVHNAKTKPREEQYASTYRVVSVLTVDHHDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHSHYTQKSLSLSPGK

SEQ ID NO: 47 - protein, 246 aa, Artificial Sequence
  Synthetic Construct
VNHKPSNTKVDKKVEPKSSDKTHTCPPCPAPELLGGPSVFLFPPKPKDTLMISRTPEVTCVVVDVSHEDPEVKFNWYVDGVEVHNAKTKPREEQYASTYRVVSVLTVDHHDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHSHYTQKSLSLSPGK

SEQ ID NO: 48 - protein, 246 aa, Artificial Sequence
  Synthetic Construct
VNHKPSNTKVDKKVEPKSSDKTHTCPPCPAPELLGGPSVFLFPPKPKDTLMISRTPEVTCVVVDVSHEDPEVKFNWYVDGVEVHNAKTKPREEQYASTYRVVSVLTVDHHDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHSHYTQKSLSLSPGK

SEQ ID NO: 49 - protein, 247 aa, Artificial Sequence; identical to SEQ ID NO: 45
  Synthetic Construct

SEQ ID NO: 50 - protein, 247 aa, Artificial Sequence; identical to SEQ ID NO: 46
  Synthetic Construct

SEQ ID NO: 51 - protein, 245 aa, Artificial Sequence
  Synthetic Construct
VNHKPSNTKVDKKVEPKSSDKTHTCPPCPAPELLGGPSVFLFPPKPKDTLMISRTPEVTCVVVDVSHEDPEVKFNWYVDGVEVHNAKTKPREEQYASTYRVVSVLTVDHHDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHSHYTQKSLSLSPG

SEQ ID NO: 52 - protein, 245 aa, Artificial Sequence
  Synthetic Construct
VNHKPSNTKVDKKVEPKSSDKTHTCPPCPAPELLGGPSVFLFPPKPKDTLMISRTPEVTCVVVDVSHEDPEVKFNWYVDGVEVHNAKTKPREEQYASTYRVVSVLTVDHHDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHSHYTQKSLSLSPG

SEQ ID NO: 53 - protein, 248 aa, Artificial Sequence
  Synthetic Construct
ISAMVRSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSEQKLISEEDL

SEQ ID NO: 54 - protein, 225 aa, Artificial Sequence
  Synthetic Construct
KTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 55 - protein, 231 aa, Artificial Sequence
  Synthetic Construct
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 56 - protein, 246 aa, Artificial Sequence
  Synthetic Construct
NVNHKPSNTKVDKKVEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 57 - protein, 246 aa, Artificial Sequence
  Synthetic Construct
NVNHKPSNTKVDKKVEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 58 - protein, 246 aa, Artificial Sequence
  Synthetic Construct
NVNHKPSNTKVDKKVEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 59 - protein, 6 aa, Artificial Sequence
  Synthetic Construct
HHHHHH

SEQ ID NO: 60 - protein, 20 aa, Homo sapiens
MYRMQLLSCIALSLALVTNS

SEQ ID NO: 61 - protein, 3 aa, Artificial Sequence
  Synthetic Construct
GGS

SEQ ID NO: 62 - protein, 5 aa, Artificial Sequence
  Synthetic Construct
GGGGS

SEQ ID NO: 63 - protein, 4 aa, Artificial Sequence
  Synthetic Construct
GGSG

SEQ ID NO: 64 - protein, 4 aa, Artificial Sequence
  Synthetic Construct
SGGG

SEQ ID NO: 65 - protein, 4 aa, Artificial Sequence
  Synthetic Construct
GSGS

SEQ ID NO: 66 - protein, 6 aa, Artificial Sequence
  Synthetic Construct
GSGSGS

SEQ ID NO: 67 - protein, 8 aa, Artificial Sequence
  Synthetic Construct
GSGSGSGS

SEQ ID NO: 68 - protein, 10 aa, Artificial Sequence
  Synthetic Construct
GSGSGSGSGS

SEQ ID NO: 69 - protein, 12 aa, Artificial Sequence
  Synthetic Construct
GSGSGSGSGSGS

SEQ ID NO: 70 - protein, 6 aa, Artificial Sequence
  Synthetic Construct
GGSGGS

SEQ ID NO: 71 - protein, 9 aa, Artificial Sequence
  Synthetic Construct
GGSGGSGGS

SEQ ID NO: 72 - protein, 12 aa, Artificial Sequence
  Synthetic Construct
GGSGGSGGSGGS

SEQ ID NO: 73 - protein, 4 aa, Artificial Sequence
  Synthetic Construct
GGSG

SEQ ID NO: 74 - protein, 8 aa, Artificial Sequence
  Synthetic Construct
GGSGGGSG

SEQ ID NO: 75 - protein, 12 aa, Artificial Sequence
  Synthetic Construct
GGSGGGSGGGSG

SEQ ID NO: 76 - protein, 15 aa, Artificial Sequence
  Synthetic Construct
GGGGSGGGGSGGGGS

SEQ ID NO: 77 - protein, 20 aa, Artificial Sequence
  Synthetic Construct
SGGGSGGGSGGGSGGGSGGG

SEQ ID NO: 78 - protein, 4 aa, Artificial Sequence
  Synthetic Construct
GGGG

SEQ ID NO: 79 - protein, 8 aa, Artificial Sequence
  Synthetic Construct
GGGGGGGG

SEQ ID NO: 80 - protein, 12 aa, Artificial Sequence
  Synthetic Construct
GGGGGGGGGGGG

SEQ ID NO: 81 - protein, 16 aa, Artificial Sequence
  Synthetic Construct
GGGGGGGGGGGGGGGG

SEQ ID NO: 82 - protein, 20 aa, Artificial Sequence
  Synthetic Construct
GGGGGGGGGGGGGGGGGGGG

SEQ ID NO: 83 - protein, 5 aa, Artificial Sequence
  Synthetic Construct
GGGGG

SEQ ID NO: 84 - protein, 10 aa, Artificial Sequence
  Synthetic Construct
GGGGGGGGGG

SEQ ID NO: 85 - protein, 15 aa, Artificial Sequence
  Synthetic Construct
GGGGGGGGGGGGGGG

SEQ ID NO: 86 - protein, 10 aa, Artificial Sequence
  Synthetic Construct
GENLYFQSGG

SEQ ID NO: 87 - protein, 8 aa, Artificial Sequence
  Synthetic Construct
SACYCELS

SEQ ID NO: 88 - protein, 5 aa, Artificial Sequence
  Synthetic Construct
RSIAT

SEQ ID NO: 89 - protein, 17 aa, Artificial Sequence
  Synthetic Construct
RPACKIPNDLKQKVMNH

SEQ ID NO: 90 - protein, 36 aa, Artificial Sequence
  Synthetic Construct
GGSAGGSGSGSSGGSSGASGTGTAGGTGSGSGTGSG

SEQ ID NO: 91 - protein, 17 aa, Artificial Sequence
  Synthetic Construct
AAANSSIDLISVPVDSR

SEQ ID NO: 92 - protein, 36 aa, Artificial Sequence
  Synthetic Construct
GGSGGGSEGGGSEGGGSEGGGSEGGGSEGGGSGGGS

(19) United States
(12) Patent Application Publication
(10) Pub. No.: US 2022/0175943 A1
(43) Pub. Date: Jun. 9, 2022

(54) VARIANT FC DOMAINS AND USES THEREOF

(71) Applicant: Cidara Therapeutics, Inc., San Diego, CA (US)

(72) Inventor: Leslie W. TARI, Solana Beach, CA (US)

(21) Appl. No.: 17/675,775

(22) Filed: Feb. 18, 2022

Related U.S. Application Data
(63) Continuation of application No. PCT/US2020/047490, filed on Aug. 21, 2020.
(60) Provisional application No. 63/062,377, filed on Aug. 6, 2020.
(60) Provisional application No. 63/032,316, filed on May 29, 2020.
(60) Provisional application No. 63/032,488, filed on May 29, 2020.
(60) Provisional application No. 62/988,821, filed on Mar. 12, 2020.
(60) Provisional application No. 62/988,304, filed on Mar. 11, 2020.
(60) Provisional application No. 62/984,705, filed on Mar. 3, 2020.
(60) Provisional application No. 62/970,491, filed on Feb. 5, 2020.
(60) Provisional application No. 62/966,500, filed on Jan. 27, 2020.
(60) Provisional application No. 62/959,857, filed on Jan. 10, 2020.
(60) Provisional application No. 62/948,143, filed on Dec. 13, 2019.
(60) Provisional application No. 62/941,405, filed on Nov. 27, 2019.
(60) Provisional application No. 62/897,036, filed on Sep. 6, 2019.
(60) Provisional application No. 62/890,475, filed on Aug. 22, 2019.

(51) Int. Cl. A61K 47/68; C07K 16/28

(52) U.S. Cl. CPC A61K 47/68; C07K 16/283

(57) ABSTRACT

This disclosure relates to variant Fc domain monomers, fusion proteins, conjugates, compositions, and related methods for treating or preventing disease. In particular, the invention features variant Fc domain monomers which include mutations at position 220, and 252, 254, and/or 256 or 309, 311, and/or 434 according to the Kabat Index numbering. The invention also features variant Fc domain monomers including mutations at position 220 according to the Kabat index number, wherein the variant Fc domain monomer is between 200 and 300 amino acid residues in length and/or is between about 20 kDa and about 40 kDa in mass.

### BACKGROUND

The utility of many therapeutics, such as small molecule therapeutic agents and biologics such as peptides, polypeptides, and polynucleotides, suffer from inadequate serum half-lives. This necessitates the administration of such therapeutics at high frequencies and/or higher doses, or the use of sustained release formulations in order to maintain the serum levels necessary for therapeutic effects. Frequent systemic administration of drugs is associated with considerable negative side effects. For example, frequent systemic injections represent a considerable discomfort to the subject, pose a high risk of administration related infections, and may require hospitalization or frequent visits to the hospital, in particular when the therapeutic is to be administered intravenously. Moreover, in long term treatments, daily intravenous injections can also lead to considerable side effects of tissue scarring and vascular pathologies caused by the repeated puncturing of vessels. Similar problems are known for all frequent systemic administrations of therapeutics. All these factors lead to a decrease in patient compliance and increased cost for the health system.

New and more effective ways of increasing therapeutic half-life and efficacy are needed.

### SUMMARY

The present disclosure provides Fc domain monomers, conjugates including an Fc domain monomer, and fusion proteins including an Fc domain monomer, wherein the Fc domain monomer is a mutational variant of a parent Fc polypeptide (e.g., an IgG1 or IgG2 polypeptide). The Fc domain monomers may include one or more mutations that contribute to increased half-life and/or efficacy. The one or more mutations may also minimize aggregation during manufacturing, thereby increasing production and lowering cost. The Fc domain monomers may also be optimized for size (e.g., as measured by kDa or amino acid residues) so as to maximize tissue distribution to a tissue or interest and/or to minimize renal clearance.

In one aspect, the disclosure provides a variant Fc domain monomer (e.g., a variant of a parent Fc polypeptide). The variant Fc domain monomer may include an amino acid substitution at position 220. The variant Fc domain monomer may include amino acid substitutions at position 220 and positions 252, 254, and 256. The variant Fc domain monomer may include amino acid substitutions at positions 309, 311, and 434. In some embodiments, the substitution at position 220 is a serine, the substitution at position 252 is a tyrosine, the substitution at position at position 254 is a threonine, the substitution at position 256 is a glutamic acid, the substitution at position 309 is an aspartic acid, the substitution at position at position 311 is a histidine, and/or the substitution at position 434 is a serine. In some embodiments, the variant Fc domain monomer includes substitutions at positions 220, 252, 254, and 256, where numbering is according to the EU index as in Kabat, and where the substitution at position 220 is a serine, the substitution at position 252 is a tyrosine, the substitution at position at position 254 is a threonine, and the substitution at position 256 is a glutamic acid. In some embodiments, the substitution at position 220 is a cysteine to serine (C220S). In some embodiments, the substitution at position 252 is a methionine to tyrosine (M252Y). In some embodiments, the substitution at position 254 is a serine to threonine (S254T). In some embodiments, the substitution at position 252 is a threonine to glutamate (T256E). In some embodiments, the substitution at position 309 is a valine to aspartic acid (V309D). In some embodiments, the substitution at position 311 is a glutamine to histidine (Q311H). In some embodiments, the substitution at position 434 is an asparagine to serine (N434S). The amino acid numbering of a variant Fc monomer as indicated above and throughout the disclosure is according to the EU index as in Kabat. Amino acid substitutions are relative to a wild-type Fc monomer amino acid sequence, e.g., wild-type human IgG1 or IgG2.

In some embodiments, the variant Fc domain monomer includes less than about 300 amino acid residues (e.g., less than about 300, less than about 295, less than about 290, less than about 285, less than about 280, less than about 275, less than about 270, less than about 265, less than about 260, less than about 255, less than about 250, less than about 245, less than about 240, less than about 235, less than about 230, less than about 225, or less than about 220 amino acid residues). In some embodiments, the variant Fc domain monomer is less than about 40 kDa (e.g., less than about 35 kDa, less than about 30 kDa, less than about 25 kDa).

In some embodiments, the variant Fc domain monomer includes at least 200 amino acid residues (e.g., at least 210, at least 220, at least 230, at least 240, at least 250, at least 260, at least 270, at least 280, at least 290, or at least 300 amino residues). In some embodiments, the variant Fc domain monomer is at least 20 kDa (e.g., at least 25 kDa, at least 30 kDa, or at least 35 kDa).

In some embodiments, the variant Fc domain monomer includes 200 to 400 amino acid residues (e.g., 200 to 250, 250 to 300, 300 to 350, 350 to 400, 200 to 300, 250 to 350, or 300 to 400 amino acid residues). In some embodiments, the variant Fc domain monomer is between 200 and 300 amino acid residues (e.g., between 210 and 300, between 230 and 300, between 250 and 300, between 270 and 300, between 290 and 300, between 210 and 290, between 220 and 280, between 230 and 270, between 240 and 260, or between 245 and 255 amino acid residues) in length. In particular embodiments, the variant Fc domain monomer is between 240 and 255 amino acid residues (e.g., 241 amino acid residues, 242 amino acid residues, 243 amino acid residues, 244 amino acid residues, 245 amino acid residues, 246 amino acid residues, 247 amino acid residues, 248 amino acid residues, 249 amino acid residues, 250 amino acid residues, 251 amino acid residues, 252 amino acid residues, 253 amino acid residues, or 254 amino acid residues). In even more particular embodiments, the variant Fc domain monomer is 246 amino acid residues in length. In some embodiments, the variant Fc domain monomer is 20 to 40 kDa (e.g., 20 to 25 kDa, 25 to 30 kDa, 35 to 40 kDa, 20 to 30 kDa, 25 to 35 kDa, or 30 to 40 KDa). In some embodiments, the variant Fc domain monomer is between about 20 kDa and about 40 kDa (e.g., 20 kDa to 25 kDa, 25 k Da to 30 k Da, 30 k Da to 35 k Da, 35 k Da to 40 kDa) in mass.

In some embodiments, the variant Fc domain monomer includes an amino acid sequence at least 90% identical (e.g., at least 95%, at least 98%) to the sequence of any one of SEQ ID NOs: 1-52 or 56-58, or a region thereof. In some embodiments, the variant Fc domain monomer includes the amino acid sequence of any one of SEQ ID NOs: 1-52 or 56-58, or a region thereof. In some embodiments, the variant Fc domain monomer includes an amino acid sequence at least 90% identical (e.g., at least 95%, at least 98%) to the sequence of any one of SEQ ID NOs: 1-19, or a region thereof. In some embodiments, the variant Fc domain monomer includes the amino acid sequence of any one of SEQ ID NOs: 1-19, or a region thereof.

In some embodiments, the variant Fc domain monomer includes a region of any one of SEQ ID NOs: 1-19, 23-29, or 31, wherein the region includes positions 220, 252, 254, and 256. In some embodiments, the region includes at least 40 amino acid residues, at least 50 amino acid residues, at least 60 amino acid residues, at least 70 amino acids residues, at least 80 amino acids residues, at least 90 amino acid residues, at least 100 amino acid residues, at least 110 amino acid residues, at least 120 amino residues, at least 130 amino acid residues, at least 140 amino acid residues, at least 150 amino acid residues, at least 160 amino acid residues, at least 170 amino acid residues, at least 180 amino acid residues, at least 190 amino acid residues, or at least 200 amino acid residues.

In some embodiments, the variant Fc domain monomer includes a region of any one of SEQ ID NOs: 31-52, wherein the region includes positions 220, 309, 311, and 434. In some embodiments, the region includes at least 215 amino acid residues, at least 220 amino acid residues, at least 225 amino acid residues, at least 230 amino acid residues, at least 235 amino acid residues, at least 240 amino acid residues, or at least 245 amino acid residues.

In another aspect, the disclosure provides a variant Fc domain monomer including a serine at amino acid position 220, wherein the amino acid numbering is according to the EU index as in Kabat, and wherein the variant Fc domain monomer is between 200 and 300 amino acid residues (e.g., between 210 and 300, between 230 and 300, between 250 and 300, between 270 and 300, between 290 and 300, between 210 and 290, between 220 and 280, between 230 and 270, between 240 and 260, or between 245 and 255 amino acid residues) in length. In some embodiments, the variant Fc domain monomer includes a serine at amino acid position 220, a tyrosine at position 252, a threonine at position 254, and/or a glutamic acid at position 256. In some embodiments, the variant Fc domain monomer includes a serine at amino acid position 220, an aspartic acid at position 309, a histidine at position 311, and/or a serine at position 434. In some embodiments, the variant Fc domain monomer further includes one or more (one, two, three, four, five, six, seven, eight, nine, ten or more) additional mutations (e.g., amino acid deletions, additions, and/or substitutions) relative to the corresponding human wild-type Fc sequence.

In another aspect, the disclosure provides a variant Fc domain monomer including a serine at amino acid position 220, wherein the amino acid numbering is according to the EU index as in Kabat, and wherein the variant Fc domain monomer is between about 20 kDa and about 40 kDa (e.g., 20 kDa to 25 kDa, 25 kDa to 30 kDa, 30 kDa to 35 kDa, 35 kDa to 40 kDa) in mass. In some embodiments, the variant Fc domain monomer includes a serine at amino acid position 220, a tyrosine at position 252, a threonine at position 254, and/or a glutamic acid at position 256. In some embodiments, the variant Fc domain monomer includes a serine at amino acid position 220, an aspartic acid at position 309, a histidine at position 311, and/or a serine at position 434.

In some embodiments, the variant Fc domain monomer is a variant of human IgG1 or human IgG2. In some embodiments, the variant Fc domain monomer is a variant of human IgG1.

In some embodiments, the N-terminus of the variant Fc domain monomer includes between 10 and 20 residues (e.g., 11, 12, 13, 14, 15, 16, 17, 18, or 19 residues) of the Fab domain. In certain embodiments, the N-terminus of the variant Fc domain monomer is any one of amino acid residues 198-205. In some embodiments, the N-terminus of the variant Fc domain monomer is amino acid residue 201 (e.g., Asn 201). In certain embodiments, the N-terminus of the variant Fc domain monomer is amino acid residue 202 (e.g., Val 202). In other embodiments, the C-terminus of the variant Fc domain monomer is any one of amino acid residues 437-447. In another embodiment, the C-terminus of the variant Fc domain monomer is amino acid residue 446 (e.g., Gly 446). In some embodiments, the C-terminus of the variant Fc domain monomer is amino acid residue 447 (e.g. Lys 447).

In some embodiments, the variant Fc domain monomer includes an amino acid sequence at least 90% identical (e.g., 91%, 92%, 93%, 94%, 95%, 96%, 97%, 98%, or 99%, or 100% identical) to the sequence of SEQ ID NOs: 1-29, 31-52, and 56-58 (e.g., SEQ ID NOs: 1-19, SEQ ID NOs: 20-29, SEQ ID NOs: 31-52, and 56-58), or a region thereof.

In another aspect, the invention provides a variant Fc domain including a dimer of variant Fc domain monomers each independently selected from any one of the variant Fc domain monomers described herein, where the variant Fc domain is between about 50 kDa and about 70 kDa (e.g., about 51 kDa, about 52 kDa, about 53 kDa, about 54 kDa, about 55 kDa, about 56 kDa, about 57 kDa, about 58 kDa, about 59 kDa, about 60 kDa, about 61 kDa, about 62 kDa, about 63 kDa, about 64 kDa, about 65 kDa, about 66 kDa, about 67 kDa, about 68 kDa, or about 69 kDa) in mass. In some embodiments, the variant Fc domain monomer dimerizes (e.g., a homodimer or a heterodimer) to form a variant Fc domain. In some embodiments, the variant Fc domain is at least 40 kDa (e.g., at least 45 kDa, at least 50 kDa, at least 55 kDa, at least 60 kDa, at least 65 kDa, at least 70 kDa, at least 75 kDa, or at least 80 kDa). In some embodiments, the variant Fc domain is between 40 kDa and 80 kDa (e.g., between about 42 kDa and about 50 kDa, about 48 kDa and about 55 kDa, about 53 kDa about 60 kDa, about 58 kDa and about 65 kDa, about 62 kDa and about 70 kDa, about 68 kDa and about 75 kDa, or about 72 kDa and about 80 kDa) in mass. In particular embodiments, the variant Fc domain is between 55 kDa and 62 kDa (e.g., about 56 kDa, about 57 kDa, about 58 kDa, about 59 kDa, about 60 kDa, or about 61 kDa). In preferred embodiments, the variant Fc domain is a homodimer including two variant Fc domain monomers (e.g., a homodimer in which each variant Fc domain monomer includes the sequence of any one of SEQ ID NOs: 1-52 or 56-58).

In another aspect, the disclosure provides a conjugate including a variant Fc domain described herein and at least one therapeutic agent, wherein the variant Fc domain monomer is covalently conjugated to the at least one therapeutic agent by a linker. In some embodiments, the conjugate is described by formula (1):

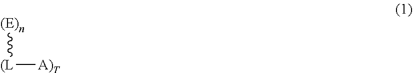

where each A is independently a therapeutic agent;
each E includes a variant Fc domain monomer or a polypeptide including a variant Fc domain monomer;
L is a linker;
n is 1 or 2;
T is an integer from 1 to 20; and
the squiggly line connected to the E indicates that each L-A is covalently attached to E (e.g., by way of a linker or a bond),
   or a pharmaceutically acceptable salt thereof.

In some embodiments, the therapeutic agent (A) is a small molecule therapeutic agent. In certain embodiments, the therapeutic agent (A) is a monomer (e.g., a single) small molecule therapeutic agent. In some embodiments, the therapeutic agent (A) is a multimer (e.g., 2 or more, 3 or more, 4 or more, or 5 or more) of small molecule therapeutic agents. In some embodiments, where (A) is a multimer (e.g., 2 or more, 3 or more, 4 or more, or 5 or more) of small molecule therapeutic agents, each of (A) can be the same small molecule agent or a different small molecule agent. In certain embodiments, where the therapeutic agent (A) is a multimer (e.g., 2 or more, 3 or more, 4 or more, or 5 or more) of small molecule agents, each of the small molecule agents are linked by any linker described herein. In some embodiments, linker that has a trivalent structure (e.g., a trivalent linker). A trivalent linker has three arms, in which each arm is covalently linked to a component of the conjugate (e.g., a first arm conjugated to a first therapeutic agent, a second arm conjugated to a second therapeutic agent, and a third arm conjugated to the fusion protein or the variant Fc domain monomer).

In some embodiments, each linker includes a polyethylene glycol (PEG) linker including between about 2-10 (e.g., 2, 3, 4, 5, 6, 7, 8, 9, or 10) PEG units. In some embodiments, at least one arm of the trivalent linker includes a polyethylene glycol (PEG) linker including between about 2-10 (e.g., 2, 3, 4, 5, 6, 7, 8, 9, or 10) PEG units.

In some embodiments, the therapeutic agent (A) is an antiviral agent, an antifungal agent, or an antibacterial agent. In some embodiments, the therapeutic agent is an antiviral agent. In some embodiments, the therapeutic agent is an antifungal agent. In further embodiments, the therapeutic agent is an antibacterial agent.

In some embodiments, the conjugate is at least 40 kDa (e.g., at least 45 kDa, at least 50 kDa, at least 55 kDa, at least 60 kDa, at least 65 kDa, at least 70 kDa, at least 75 kDa, or at least 80 kDa). In some embodiments, the conjugate is between about 40 kDa and about 80 kDa (e.g., 40 kDa to 50 kDa, 45 kDa to 55 kDa, 50 kDa to 60 kDa, 55 kDa to 65 kDa, 60 kDa to 70 kDa, 65 kDa to 75 kDa, or 70 kDa to 80 kDa) in mass. In particular embodiments, the conjugate is between 58 kDa and 70 kDa (e.g., about 59 kDa, about 60 kDa, or about 61 kDa, 62 kDa, 63 kDa, 64 kDa, 65 kDa, 66 kDa, 67 kDa, 68 kDa, or 69 kDa) in mass.

In another aspect, the disclosure provides a fusion protein comprising a variant Fc domain monomer and at least one polypeptide therapeutic agent, wherein the variant Fc domain monomer is covalently conjugated to the polypeptide therapeutic agent by a linker. In some embodiments, the fusion protein includes the structure:

(P₂-L₂)ₙ₂-B-(L₁-P₁)ₙ₁
wherein B is a variant Fc domain monomer, a polypeptide including a variant Fc domain monomer, or a conjugate (e.g., any conjugate described herein); P₁ and P₂ are each independently a polypeptide therapeutic agent; L₁ and L₂ are each independently a linker; and n₁ and n₂ are each independently 0 or 1, wherein at least one of n₁ and n₂ is 1.

In some embodiments, the fusion protein includes less than about 500 amino acid residues (e.g., less than about 495, less than about 490, less than about 485, less than about 480, less than about 475, less than about 470, less than about 465, less than about 460, less than about 455, less than about 450, less than about 445, less than about 440, less than about 435, less than about 430, less than about 425, less than about 420, less than about 415, less than about 410, less than about 405, less than about 400, less than about 395, less than about 390, less than about 385, less than about 380, less than about 375, less than about 370, less than about 365, less than about 360, less than about 355, less than about 350, less than about 345, less than about 340, less than about 335, less than about 330, less than about 325, less than about 320, less than about 315, less than about 310, less than about 305, less than about 300, less than about 295, less than about 290, less than about 285, less than about 280, less than about 275, less than about 270, less than about 265, less than about 260, or less than about 255). In some embodiments, the variant Fc domain monomer is less than about 50 kDa (e.g., less than about 45 kDa, less than about 40 kDa, less than about 35 kDa, or less than about 30 kDa).

In some embodiments, the fusion protein includes at least 250 amino acid residues (e.g., at least about 250, at least about 260, at least about 270, at least about 280, at least about 290, at least about 300 amino residues, at least about 310, at least about 320, at least about 330, at least about 340, at least about 350, at least about 360, at least about 370, at least about 380, at least about 390, at least about 400, at least about 410, at least about 420, at least about 430, at least about 440, at least about 450, at least about 460, at least about 470, at least about 480, or at least about 490). In some embodiments, the fusion protein is at least about 30 kDa (e.g., at least at least about 35 kDa, at least at least about 40 kDa, or at least at least about 45).

In some embodiments, the fusion protein includes 250 to 500 amino acid residues (e.g., 250 to 300, 300 to 350, 350 to 400, 200 to 300, 250 to 350, 300 to 400, 350 to 450, or 400 to 500 amino acid residues). In some embodiments, the variant Fc domain monomer is 30 to 50 kDa (e.g., 30 to 35 kDa, 30 to 40 kDa, 35 to 45 kDa, or 40 to 50 kDa).

In some embodiments, the therapeutic polypeptides each independently include less than about 200 amino acid residues (e.g., less than about 195, less than about 190, less than about 185, less than about 180, less than about 175, less than about 170, less than about 165, less than about 160, less than about 155, less than about 150, less than about 145, less than about 140, less than about 135, less than about 130, less than about 125, less than about 120, less than about 115, less than about 110, less than about 105, less than about 100, less than about 95, less than about 90, less than about 85, less than about 80, less than about 75, less than about 70, less than about 65, less than about 60, less than about 55, less than about 50, less than about 45, less than about 40, less than about 35, less than about 30, less than about 25, less than about 20, or less than about 15 amino acid residues).

In some embodiments, the therapeutic polypeptides each independently include at least about 10 amino acid residues (e.g., at least about 15, at least about 20, at least about 25, at least about 30, at least about 35, at least about 40, at least about 45, at least about 50, at least about 55, at least about 60, at least about 65, at least about 70, at least about 75, at least about 80, at least about 85, at least about 90, at least about 95 amino acid residues, at least about 100, at least about 105, at least about 110, at least about 115, at least about 120, at least about 125, at least about 130, at least about 135, at least about 140, at least about 145, at least about 150, at least about 155, at least about 160, at least about 165, at least about 170, at least about 175, at least about 180, at least about 185, at least about 190, or at least about 195 amino acid residues).

In some embodiments, n₁ is 1, n₂ is 0, and the fusion protein includes the structure:

B-L₁-P₁.

In some embodiments the linker (L₁) is conjugated to C-terminus of the Fc domain monomer (B) and to the N-terminus of the polypeptide therapeutic agent (P₁). In some embodiments, the linker (L₁) is conjugated to N-terminus of the Fc domain monomer (B) and to the C-terminus of the polypeptide therapeutic agent (P₁). In some embodiments, L₁ is a peptide linker including between 2 and 200 amino acids. In some embodiments, L₁ is a peptide linker including between 5 and 25 amino acids. In some embodiments, L₁ is a peptide linker including the amino acid sequence of any one of (GS)ₓ, (GGS)ₓ, (GGGGS)ₓ, (GGSG)ₓ, (SGGG)ₓ, wherein x is an integer from 1 to 10. In some embodiments, when B, L₁, and P₁ are expressed as a single polypeptide chain. In some embodiments, the linker (L₁) is conjugated to N-terminus of the Fc domain monomer (B) and to the N-terminus of the polypeptide therapeutic agent (P₁). In some embodiments, the linker (L₁) is conjugated to C-terminus of the Fc domain monomer (B) and to the C-terminus of the polypeptide therapeutic agent (P₁). In some embodiments, L₁ includes a chemical linker that is covalently conjugated to each of B and P₁. In some embodiments, B and P₁ are expressed as separate polypeptide chains and are subsequently each covalently conjugated to L₁.

In some embodiments, n₁ is 1, n₂ is 1, and the fusion protein includes the structure:

P₂-L₂-B-L₁-P₁.

In some embodiments, the linker (L₂) is conjugated to the C-terminus of the polypeptide therapeutic agent (P₂) and to the N-terminus of the Fc domain monomer (B), and the linker (L₁) is conjugated to the C-terminus of the Fc domain monomer (B) and to the N-terminus of the polypeptide therapeutic agent (P₁). In some embodiments, L₁ and L₂ are each an independently selected peptide linker including between 2 and 200 amino acids. In some embodiments, L₁ and L₂ are each an independently selected peptide linker including between 5 and 25 amino acids. In some embodiments, L₁ and L₂ are each an independently selected peptide linker including the amino acid sequence of any one of (GS)ₓ, (GGS)ₓ, (GGGGS)ₓ, (GGSG)ₓ, (SGGG)ₓ, wherein x is an integer from 1 to 10 (e.g., 1, 2, 3, 4, 5, 6, 7, 8, 9, or 10). In some embodiments, P₂, L₂, B, L₁, and P₁ are expressed together as a single polypeptide chain. In some embodiments, the linker (L₂) is conjugated to the N-terminus of the polypeptide therapeutic agent (P₂) and to the N-terminus of the Fc domain monomer (B), and the linker (L₁) is conjugated to the N-terminus of the polypeptide therapeutic agent (P₁) and to the C-terminus of the Fc domain monomer (B). In some embodiments, the linker (L₂) is conjugated to the C-terminus of the polypeptide therapeutic agent (P₂) and to the N-terminus of the Fc domain monomer (B), and the linker (L₁) is conjugated to the C-terminus of the polypeptide therapeutic agent (P₁) and to the C-terminus of the Fc domain monomer (B). In some embodiments, L₂ includes a chemical linker that is covalently conjugated to each of B and P₂, and L₁ includes a chemical linker that is covalently conjugated to each of B and P₁. In some embodiments, P₂, B, and P₁ are expressed as separate polypeptide chains, P₂ and B are subsequently each covalently conjugated to L₂, and P₁ and B are subsequently each covalently conjugated to L₁.

In some embodiments of any aspect described herein, the variant Fc domain monomer dimerizes to form an Fc domain. In some embodiments, each of the variant Fc domain monomers in an Fc domain have the same amino acid sequence, thereby forming a homodimer Fc domain.

In another aspect, the disclosure provides, a pharmaceutical composition including any variant Fc domain monomer described herein, any conjugate described herein, any fusion protein described herein, or any Fc domain, and a pharmaceutically acceptable carrier.

In another aspect, the disclosure provides a method of treating or preventing a respiratory disorder in a subject, the method including administering to the subject any composition described herein. In some embodiments, the respiratory disorder is an infection. In some embodiments, the infection is a viral infection. In some embodiments, the viral infection is selected from the group including RSV, Influenza, Dengue, a beta coronavirus (e.g., COVID-19), and Zika virus. In some embodiments, the infection is a bacterial infection. In some embodiments, the respiratory disorder is selected from the group including chronic obstructive pulmonary disease (COPD), chronic bronchitis, cystic fibrosis, bronchiectasis, and pneumonia.

In some embodiments, a ratio of the concentration of the Fc domain monomer, the conjugate, the fusion protein, or Fc domain in epithelial lining fluid is at least 30% of the concentration of the Fc domain monomer, the conjugate, the fusion protein, or the Fc domain in plasma within 2 hours after administration. In some embodiments, the ratio of the concentration is at least 45% within 2 hours after administration. In some embodiments, the ratio of concentration is at least 55% within 2 hours after administration. In some embodiments, the ratio of concentration is at least 60% within 2 hours after administration. In particular embodiments of the above, the route of administration is by injection, e.g., by intramuscular, subcutaneous, intraperitoneal, or intravenous injection. In particular embodiments of the above, the route of administration is oral.

In another aspect, the disclosure provides a method of treating or preventing a hepatic disorder in a subject, the method including administering to the subject any composition described herein. In some embodiments, the hepatic disorder is an infection (e.g., a viral infection, such as Hepatitis A, Hepatitis B, or Hepatitis C), a fungal infection or a bacterial infection. In some embodiments, the hepatic disorder is selected from the group including primary biliary cholangitis, primary sclerosing cholangitis, hepatocellular carcinoma, bile duct cancer, liver cell adenoma, nonalcoholic fatty liver disease (NAFLD), acute liver failure, and cirrhosis.

In another aspect, the disclosure provides a method of treating or preventing a central nervous system (CNS) disorder in a subject, the method including administering to the subject any composition described herein. In some embodiments, the CNS disorder is an infection. In some embodiments, the infection is a viral infection, a bacterial infection, or a fungal infection. In some embodiments, the viral infection is selected from the group including herpes simplex virus (HSV) 1, HSV 2, Epstein-Barr virus, varicella-zoster virus, poliovirus, coxsackievirus, West Nile virus, Lacrosse virus, western equine encephalitis, eastern equine encephalitis, Powassan virus, or rabies virus. In some embodiments, the CNS disorder is selected from the group including cancer, Alzheimer disease, Parkinson disease, epilepsy, multiple sclerosis, schizophrenia, and meningitis.

In another aspect, the disclosure provides a method of treating or preventing a muscle disorder in a subject, the method including administering to the subject any composition described herein. In some embodiments, the muscle disorder is myositis or cancer. In some embodiments, the myositis is caused by an injury, an infection, or an immune disorder.

In another aspect, the disclosure provides a method of treating or preventing a skin disorder in a subject, the method including administering to the subject any composition described herein. In some embodiments, the skin disorder is an infection (e.g., a viral infection (HSV 1, HSV 2, or varicella-zoster virus), a fungal infection, or a bacterial infection. In some embodiments, the skin disorder is selected from the group including eczema, psoriasis, acne, rosacea, cold sores, cellulitis, basal cell carcinoma, squamous cell carcinoma, and melanoma.

In another aspect, the disclosure provides method of treating or preventing an ocular disorder in a subject, the method including administering to the subject any composition described herein. In some embodiments, the ocular disorder is an infection (e.g., a viral infection (HSV 1 or HSV 2), a fungal infection, or a bacterial infection. In some embodiments, the ocular disorder is selected from age-related macular degeneration, cataract, and glaucoma.

In another aspect, the disclosure provides a method of treating or preventing a vascular disorder in a subject, including administering to the subject any composition described herein. In some embodiments, the vascular disorder is an infection (e.g., a viral infection, a fungal infection, or a bacterial infection).

In some embodiments of any of the aspects described herein, the variant Fc domain monomer (e.g., each variant Fc domain monomer of an Fc domain) includes the amino acid sequence of SEQ ID NO: 1. In some embodiments, the variant Fc domain monomer includes an amino acid sequence that is at least 70%, 75%, 80%, 85%, 90%, 95%, 96%, 97%, 98%, 99%, or 100% identical to the amino acid sequence of SEQ ID NO: 1.

In some embodiments of any of the aspects described herein, the variant Fc domain monomer (e.g., each variant Fc domain monomer of an Fc domain) includes the amino acid sequence of SEQ ID NO: 2. In some embodiments, the variant Fc domain monomer includes an amino acid sequence that is at least 70%, 75%, 80%, 85%, 90%, 95%, 96%, 97%, 98%, 99%, or 100% identical to the amino acid sequence of SEQ ID NO: 2.

In some embodiments of any of the aspects described herein, the variant Fc domain monomer (e.g., each variant Fc domain monomer of an Fc domain) includes the amino acid sequence of SEQ ID NO: 3. In some embodiments, the variant Fc domain monomer includes an amino acid sequence that is at least 70%, 75%, 80%, 85%, 90%, 95%, 96%, 97%, 98%, 99%, or 100% identical to the amino acid sequence of SEQ ID NO: 3.

In some embodiments of any of the aspects described herein, the variant Fc domain monomer (e.g., each variant Fc domain monomer of an Fc domain) includes the amino acid sequence of SEQ ID NO: 4. In some embodiments, the variant Fc domain monomer includes an amino acid sequence that is at least 70%, 75%, 80%, 85%, 90%, 95%, 96%, 97%, 98%, 99%, or 100% identical to the amino acid sequence of SEQ ID NO: 4.

In some embodiments of any of the aspects described herein, the variant Fc domain monomer (e.g., each variant Fc domain monomer of an Fc domain) includes the amino acid sequence of SEQ ID NO: 5. In some embodiments, the variant Fc domain monomer includes an amino acid sequence that is at least 70%, 75%, 80%, 85%, 90%, 95%, 96%, 97%, 98%, 99%, or 100% identical to the amino acid sequence of SEQ ID NO: 5.

In some embodiments of any of the aspects described herein, the variant Fc domain monomer (e.g., each variant Fc domain monomer of an Fc domain) includes the amino acid sequence of SEQ ID NO: 6. In some embodiments, the variant Fc domain monomer includes an amino acid sequence that is at least 70%, 75%, 80%, 85%, 90%, 95%, 96%, 97%, 98%, 99%, or 100% identical to the amino acid sequence of SEQ ID NO: 6.

In some embodiments of any of the aspects described herein, the variant Fc domain monomer (e.g., each variant Fc domain monomer of an Fc domain) includes the amino acid sequence of SEQ ID NO: 8. In some embodiments, the variant Fc domain monomer includes an amino acid sequence that is at least 70%, 75%, 80%, 85%, 90%, 95%, 96%, 97%, 98%, 99%, or 100% identical to the amino acid sequence of SEQ ID NO: 8.

In some embodiments of any of the aspects described herein, the variant Fc domain monomer (e.g., each variant Fc domain monomer of an Fc domain) includes the amino acid sequence of SEQ ID NO: 9. In some embodiments, the variant Fc domain monomer includes an amino acid sequence that is at least 70%, 75%, 80%, 85%, 90%, 95%, 96%, 97%, 98%, 99%, or 100% identical to the amino acid sequence of SEQ ID NO: 9.

In some embodiments of any of the aspects described herein, the variant Fc domain monomer (e.g., each variant Fc domain monomer of an Fc domain) includes the amino acid sequence of SEQ ID NO: 10. In some embodiments, the variant Fc domain monomer includes an amino acid sequence that is at least 70%, 75%, 80%, 85%, 90%, 95%, 96%, 97%, 98%, 99%, or 100% identical to the amino acid sequence of SEQ ID NO: 10.

In some embodiments of any of the aspects described herein, the variant Fc domain monomer (e.g., each variant Fc domain monomer of an Fc domain) includes the amino acid sequence of SEQ ID NO: 11. In some embodiments, the variant Fc domain monomer includes an amino acid sequence that is at least 70%, 75%, 80%, 85%, 90%, 95%, 96%, 97%, 98%, 99%, or 100% identical to the amino acid sequence of SEQ ID NO: 11.

In some embodiments of any of the aspects described herein, the variant Fc domain monomer (e.g., each variant Fc domain monomer of an Fc domain) includes the amino acid sequence of SEQ ID NO: 12. In some embodiments, the variant Fc domain monomer includes an amino acid sequence that is at least 70%, 75%, 80%, 85%, 90%, 95%, 96%, 97%, 98%, 99%, or 100% identical to the amino acid sequence of SEQ ID NO: 12.

In some embodiments of any of the aspects described herein, the variant Fc domain monomer (e.g., each variant Fc domain monomer of an Fc domain) includes the amino acid sequence of SEQ ID NO: 13. In some embodiments, the variant Fc domain monomer includes an amino acid sequence that is at least 70%, 75%, 80%, 85%, 90%, 95%, 96%, 97%, 98%, 99%, or 100% identical to the amino acid sequence of SEQ ID NO: 13.

In some embodiments of any of the aspects described herein, the variant Fc domain monomer (e.g., each variant Fc domain monomer of an Fc domain) includes the amino acid sequence of SEQ ID NO: 14. In some embodiments, the variant Fc domain monomer includes an amino acid sequence that is at least 70%, 75%, 80%, 85%, 90%, 95%, 96%, 97%, 98%, 99%, or 100% identical to the amino acid sequence of SEQ ID NO: 14.

In some embodiments of any of the aspects described herein, the variant Fc domain monomer (e.g., each variant Fc domain monomer of an Fc domain) includes the amino acid sequence of SEQ ID NO: 15. In some embodiments, the variant Fc domain monomer includes an amino acid sequence that is at least 70%, 75%, 80%, 85%, 90%, 95%, 96%, 97%, 98%, 99%, or 100% identical to the amino acid sequence of SEQ ID NO: 15.

In some embodiments of any of the aspects described herein, the variant Fc domain monomer (e.g., each variant Fc domain monomer of an Fc domain) includes the amino acid sequence of SEQ ID NO: 16. In some embodiments, the variant Fc domain monomer includes an amino acid sequence that is at least 70%, 75%, 80%, 85%, 90%, 95%, 96%, 97%, 98%, 99%, or 100% identical to the amino acid sequence of SEQ ID NO: 16.

In some embodiments of any of the aspects described herein, the variant Fc domain monomer (e.g., each variant Fc domain monomer of an Fc domain) includes the amino acid sequence of SEQ ID NO: 17. In some embodiments, the variant Fc domain monomer includes an amino acid sequence that is at least 70%, 75%, 80%, 85%, 90%, 95%, 96%, 97%, 98%, 99%, or 100% identical to the amino acid sequence of SEQ ID NO: 17.

In some embodiments of any of the aspects described herein, the variant Fc domain monomer (e.g., each variant Fc domain monomer of an Fc domain) includes the amino acid sequence of SEQ ID NO: 18. In some embodiments, the variant Fc domain monomer includes an amino acid sequence that is at least 70%, 75%, 80%, 85%, 90%, 95%, 96%, 97%, 98%, 99%, or 100% identical to the amino acid sequence of SEQ ID NO: 18.

In some embodiments of any of the aspects described herein, the variant Fc domain monomer (e.g., each variant Fc domain monomer of an Fc domain) includes the amino acid sequence of SEQ ID NO: 19. In some embodiments, the variant Fc domain monomer includes an amino acid sequence that is at least 70%, 75%, 80%, 85%, 90%, 95%, 96%, 97%, 98%, 99%, or 100% identical to the amino acid sequence of SEQ ID NO: 19.

In some embodiments of any of the aspects described herein, the variant Fc domain monomer (e.g., each variant Fc domain monomer of an Fc domain) includes the amino acid sequence of SEQ ID NO: 20. In some embodiments, the variant Fc domain monomer includes an amino acid sequence that is at least 70%, 75%, 80%, 85%, 90%, 95%, 96%, 97%, 98%, 99%, or 100% identical to the amino acid sequence of SEQ ID NO: 20.

In some embodiments of any of the aspects described herein, the variant Fc domain monomer (e.g., each variant Fc domain monomer of an Fc domain) includes the amino acid sequence of SEQ ID NO: 21. In some embodiments, the variant Fc domain monomer includes an amino acid sequence that is at least 70%, 75%, 80%, 85%, 90%, 95%, 96%, 97%, 98%, 99%, or 100% identical to the amino acid sequence of SEQ ID NO: 21.

In some embodiments of any of the aspects described herein, the variant Fc domain monomer (e.g., each variant Fc domain monomer of an Fc domain) includes the amino acid sequence of SEQ ID NO: 22. In some embodiments, the variant Fc domain monomer includes an amino acid sequence that is at least 70%, 75%, 80%, 85%, 90%, 95%, 96%, 97%, 98%, 99%, or 100% identical to the amino acid sequence of SEQ ID NO: 22.

In some embodiments of any of the aspects described herein, the variant Fc domain monomer (e.g., each variant Fc domain monomer of an Fc domain) includes the amino acid sequence of SEQ ID NO: 23. In some embodiments, the variant Fc domain monomer includes an amino acid sequence that is at least 70%, 75%, 80%, 85%, 90%, 95%, 96%, 97%, 98%, 99%, or 100% identical to the amino acid sequence of SEQ ID NO: 23.

In some embodiments of any of the aspects described herein, the variant Fc domain monomer (e.g., each variant Fc domain monomer of an Fc domain) includes the amino acid sequence of SEQ ID NO: 24. In some embodiments, the variant Fc domain monomer includes an amino acid sequence that is at least 70%, 75%, 80%, 85%, 90%, 95%, 96%, 97%, 98%, 99%, or 100% identical to the amino acid sequence of SEQ ID NO: 24.

In some embodiments of any of the aspects described herein, the variant Fc domain monomer (e.g., each variant Fc domain monomer of an Fc domain) includes the amino acid sequence of SEQ ID NO: 25. In some embodiments, the variant Fc domain monomer includes an amino acid sequence that is at least 70%, 75%, 80%, 85%, 90%, 95%, 96%, 97%, 98%, 99%, or 100% identical to the amino acid sequence of SEQ ID NO: 25.

In some embodiments of any of the aspects described herein, the variant Fc domain monomer (e.g., each variant Fc domain monomer of an Fc domain) includes the amino acid sequence of SEQ ID NO: 26. In some embodiments, the variant Fc domain monomer includes an amino acid sequence that is at least 70%, 75%, 80%, 85%, 90%, 95%, 96%, 97%, 98%, 99%, or 100% identical to the amino acid sequence of SEQ ID NO: 26.

In some embodiments of any of the aspects described herein, the variant Fc domain monomer (e.g., each variant Fc domain monomer of an Fc domain) includes the amino acid sequence of SEQ ID NO: 27. In some embodiments, the variant Fc domain monomer includes an amino acid sequence that is at least 70%, 75%, 80%, 85%, 90%, 95%, 96%, 97%, 98%, 99%, or 100% identical to the amino acid sequence of SEQ ID NO: 27.

In some embodiments of any of the aspects described herein, the variant Fc domain monomer (e.g., each variant Fc domain monomer of an Fc domain) includes the amino acid sequence of SEQ ID NO: 28. In some embodiments, the variant Fc domain monomer includes an amino acid sequence that is at least 70%, 75%, 80%, 85%, 90%, 95%, 96%, 97%, 98%, 99%, or 100% identical to the amino acid sequence of SEQ ID NO: 28.

In some embodiments of any of the aspects described herein, the variant Fc domain monomer (e.g., each variant Fc domain monomer of an Fc domain) includes the amino acid sequence of SEQ ID NO: 29. In some embodiments, the variant Fc domain monomer includes an amino acid sequence that is at least 70%, 75%, 80%, 85%, 90%, 95%, 96%, 97%, 98%, 99%, or 100% identical to the amino acid sequence of SEQ ID NO: 29.

In some embodiments of any of the aspects described herein, the variant Fc domain monomer (e.g., each variant Fc domain monomer of an Fc domain) includes the amino acid sequence of SEQ ID NO: 30. In some embodiments, the variant Fc domain monomer includes an amino acid sequence that is at least 70%, 75%, 80%, 85%, 90%, 95%, 96%, 97%, 98%, 99%, or 100% identical to the amino acid sequence of SEQ ID NO: 30.

In some embodiments of any of the aspects described herein, the variant Fc domain monomer (e.g., each variant Fc domain monomer of an Fc domain) includes the amino acid sequence of SEQ ID NO: 31. In some embodiments, the variant Fc domain monomer includes an amino acid sequence that is at least 70%, 75%, 80%, 85%, 90%, 95%, 96%, 97%, 98%, 99%, or 100% identical to the amino acid sequence of SEQ ID NO: 31.

In some embodiments of any of the aspects described herein, the variant Fc domain monomer (e.g., each variant Fc domain monomer of an Fc domain) includes the amino acid sequence of SEQ ID NO: 32. In some embodiments, the variant Fc domain monomer includes an amino acid sequence that is at least 70%, 75%, 80%, 85%, 90%, 95%, 96%, 97%, 98%, 99%, or 100% identical to the amino acid sequence of SEQ ID NO: 32.

In some embodiments of any of the aspects described herein, the variant Fc domain monomer (e.g., each variant Fc domain monomer of an Fc domain) includes the amino acid sequence of SEQ ID NO: 33. In some embodiments, the variant Fc domain monomer includes an amino acid sequence that is at least 70%, 75%, 80%, 85%, 90%, 95%, 96%, 97%, 98%, 99%, or 100% identical to the amino acid sequence of SEQ ID NO: 33.

In some embodiments of any of the aspects described herein, the variant Fc domain monomer (e.g., each variant Fc domain monomer of an Fc domain) includes the amino acid sequence of SEQ ID NO: 34. In some embodiments, the variant Fc domain monomer includes an amino acid sequence that is at least 70%, 75%, 80%, 85%, 90%, 95%, 96%, 97%, 98%, 99%, or 100% identical to the amino acid sequence of SEQ ID NO: 34.

In some embodiments of any of the aspects described herein, the variant Fc domain monomer (e.g., each variant Fc domain monomer of an Fc domain) includes the amino acid sequence of SEQ ID NO: 35. In some embodiments, the variant Fc domain monomer includes an amino acid sequence that is at least 70%, 75%, 80%, 85%, 90%, 95%, 96%, 97%, 98%, 99%, or 100% identical to the amino acid sequence of SEQ ID NO: 35.

In some embodiments of any of the aspects described herein, the variant Fc domain monomer (e.g., each variant Fc domain monomer of an Fc domain) includes the amino acid sequence of SEQ ID NO: 36. In some embodiments, the variant Fc domain monomer includes an amino acid sequence that is at least 70%, 75%, 80%, 85%, 90%, 95%, 96%, 97%, 98%, 99%, or 100% identical to the amino acid sequence of SEQ ID NO: 36.

In some embodiments of any of the aspects described herein, the variant Fc domain monomer (e.g., each variant Fc domain monomer of an Fc domain) includes the amino acid sequence of SEQ ID NO: 37. In some embodiments, the variant Fc domain monomer includes an amino acid sequence that is at least 70%, 75%, 80%, 85%, 90%, 95%, 96%, 97%, 98%, 99%, or 100% identical to the amino acid sequence of SEQ ID NO: 37.

In some embodiments of any of the aspects described herein, the variant Fc domain monomer (e.g., each variant Fc domain monomer of an Fc domain) includes the amino acid sequence of SEQ ID NO: 38. In some embodiments, the variant Fc domain monomer includes an amino acid sequence that is at least 70%, 75%, 80%, 85%, 90%, 95%, 96%, 97%, 98%, 99%, or 100% identical to the amino acid sequence of SEQ ID NO: 38.

In some embodiments of any of the aspects described herein, the variant Fc domain monomer (e.g., each variant Fc domain monomer of an Fc domain) includes the amino acid sequence of SEQ ID NO: 39. In some embodiments, the variant Fc domain monomer includes an amino acid sequence that is at least 70%, 75%, 80%, 85%, 90%, 95%, 96%, 97%, 98%, 99%, or 100% identical to the amino acid sequence of SEQ ID NO: 39.

In some embodiments of any of the aspects described herein, the variant Fc domain monomer (e.g., each variant Fc domain monomer of an Fc domain) includes the amino acid sequence of SEQ ID NO: 40. In some embodiments, the variant Fc domain monomer includes an amino acid sequence that is at least 70%, 75%, 80%, 85%, 90%, 95%, 96%, 97%, 98%, 99%, or 100% identical to the amino acid sequence of SEQ ID NO: 40.

In some embodiments of any of the aspects described herein, the variant Fc domain monomer (e.g., each variant Fc domain monomer of an Fc domain) includes the amino acid sequence of SEQ ID NO: 41. In some embodiments, the variant Fc domain monomer includes an amino acid sequence that is at least 70%, 75%, 80%, 85%, 90%, 95%, 96%, 97%, 98%, 99%, or 100% identical to the amino acid sequence of SEQ ID NO: 41.

In some embodiments of any of the aspects described herein, the variant Fc domain monomer (e.g., each variant Fc domain monomer of an Fc domain) includes the amino acid sequence of SEQ ID NO: 42. In some embodiments, the variant Fc domain monomer includes an amino acid sequence that is at least 70%, 75%, 80%, 85%, 90%, 95%, 96%, 97%, 98%, 99%, or 100% identical to the amino acid sequence of SEQ ID NO: 42.

In some embodiments of any of the aspects described herein, the variant Fc domain monomer (e.g., each variant Fc domain monomer of an Fc domain) includes the amino acid sequence of SEQ ID NO: 43. In some embodiments, the variant Fc domain monomer includes an amino acid sequence that is at least 70%, 75%, 80%, 85%, 90%, 95%, 96%, 97%, 98%, 99%, or 100% identical to the amino acid sequence of SEQ ID NO: 43.

In some embodiments of any of the aspects described herein, the variant Fc domain monomer (e.g., each variant Fc domain monomer of an Fc domain) includes the amino acid sequence of SEQ ID NO: 44. In some embodiments, the variant Fc domain monomer includes an amino acid sequence that is at least 70%, 75%, 80%, 85%, 90%, 95%, 96%, 97%, 98%, 99%, or 100% identical to the amino acid sequence of SEQ ID NO: 44.

In some embodiments of any of the aspects described herein, the variant Fc domain monomer (e.g., each variant Fc domain monomer of an Fc domain) includes the amino acid sequence of SEQ ID NO: 45. In some embodiments, the variant Fc domain monomer includes an amino acid sequence that is at least 70%, 75%, 80%, 85%, 90%, 95%, 96%, 97%, 98%, 99%, or 100% identical to the amino acid sequence of SEQ ID NO: 45.

In some embodiments of any of the aspects described herein, the variant Fc domain monomer (e.g., each variant Fc domain monomer of an Fc domain) includes the amino acid sequence of SEQ ID NO: 46. In some embodiments, the variant Fc domain monomer includes an amino acid sequence that is at least 70%, 75%, 80%, 85%, 90%, 95%, 96%, 97%, 98%, 99%, or 100% identical to the amino acid sequence of SEQ ID NO: 46.

In some embodiments of any of the aspects described herein, the variant Fc domain monomer (e.g., each variant Fc domain monomer of an Fc domain) includes the amino acid sequence of SEQ ID NO: 47. In some embodiments, the variant Fc domain monomer includes an amino acid sequence that is at least 70%, 75%, 80%, 85%, 90%, 95%, 96%, 97%, 98%, 99%, or 100% identical to the amino acid sequence of SEQ ID NO: 47.

In some embodiments of any of the aspects described herein, the variant Fc domain monomer (e.g., each variant Fc domain monomer of an Fc domain) includes the amino acid sequence of SEQ ID NO: 48. In some embodiments, the variant Fc domain monomer includes an amino acid sequence that is at least 70%, 75%, 80%, 85%, 90%, 95%, 96%, 97%, 98%, 99%, or 100% identical to the amino acid sequence of SEQ ID NO: 48.

In some embodiments of any of the aspects described herein, the variant Fc domain monomer (e.g., each variant Fc domain monomer of an Fc domain) includes the amino acid sequence of SEQ ID NO: 49. In some embodiments, the variant Fc domain monomer includes an amino acid sequence that is at least 70%, 75%, 80%, 85%, 90%, 95%, 96%, 97%, 98%, 99%, or 100% identical to the amino acid sequence of SEQ ID NO: 49.

In some embodiments of any of the aspects described herein, the variant Fc domain monomer (e.g., each variant Fc domain monomer of an Fc domain) includes the amino acid sequence of SEQ ID NO: 50. In some embodiments, the variant Fc domain monomer includes an amino acid sequence that is at least 70%, 75%, 80%, 85%, 90%, 95%, 96%, 97%, 98%, 99%, or 100% identical to the amino acid sequence of SEQ ID NO: 50.

In some embodiments of any of the aspects described herein, the variant Fc domain monomer (e.g., each variant Fc domain monomer of an Fc domain) includes the amino acid sequence of SEQ ID NO: 51. In some embodiments, the variant Fc domain monomer includes an amino acid sequence that is at least 70%, 75%, 80%, 85%, 90%, 95%, 96%, 97%, 98%, 99%, or 100% identical to the amino acid sequence of SEQ ID NO: 51.

In some embodiments of any of the aspects described herein, the variant Fc domain monomer (e.g., each variant Fc domain monomer of an Fc domain) includes the amino acid sequence of SEQ ID NO: 52. In some embodiments, the variant Fc domain monomer includes an amino acid sequence that is at least 70%, 75%, 80%, 85%, 90%, 95%, 96%, 97%, 98%, 99%, or 100% identical to the amino acid sequence of SEQ ID NO: 52.

In some embodiments of any of the aspects described herein, the variant Fc domain monomer (e.g., each variant Fc domain monomer of an Fc domain) includes the amino acid sequence of SEQ ID NO: 56. In some embodiments, the variant Fc domain monomer includes an amino acid sequence that is at least 70%, 75%, 80%, 85%, 90%, 95%, 96%, 97%, 98%, 99%, or 100% identical to the amino acid sequence of SEQ ID NO: 56.

In some embodiments of any of the aspects described herein, the variant Fc domain monomer (e.g., each variant Fc domain monomer of an Fc domain) includes the amino acid sequence of SEQ ID NO: 57. In some embodiments, the variant Fc domain monomer includes an amino acid sequence that is at least 70%, 75%, 80%, 85%, 90%, 95%, 96%, 97%, 98%, 99%, or 100% identical to the amino acid sequence of SEQ ID NO: 57.

In some embodiments of any of the aspects described herein, the variant Fc domain monomer (e.g., each variant Fc domain monomer of an Fc domain) includes the amino acid sequence of SEQ ID NO: 58. In some embodiments, the variant Fc domain monomer includes an amino acid sequence that is at least 70%, 75%, 80%, 85%, 90%, 95%, 96%, 97%, 98%, 99%, or 100% identical to the amino acid sequence of SEQ ID NO: 58.

### Definitions

To facilitate the understanding of this invention, a number of terms are defined below. Terms defined herein have meanings as commonly understood by a person of ordinary skill in the areas relevant to the present invention. Terms such as “a”, “an,” and “the” are not intended to refer to only a singular entity, but include the general class of which a specific example may be used for illustration. The terminology herein is used to describe specific embodiments of the invention, but their usage does not delimit the invention, except as outlined in the claims.

As used herein, the term “variant Fc domain monomer,” refers to a polypeptide chain that includes at least a hinge domain and second and third antibody constant domains (C_{H}2 and C_{H}3) or functional fragments thereof (e.g., fragments that are capable of (i) dimerizing with another variant Fc domain monomer to form a variant Fc domain, and (ii) binding to an Fc receptor). In some embodiments, the variant Fc domain monomer includes, at least, the following quadruple mutation C220S/M252Y/S254T/T256E. In some embodiments, the variant Fc domain monomer includes, at least, the quadruple mutation C220S/V309D/Q311H/N434S. In some embodiments, the variant Fc domain monomer has a mutation including C220S. A variant Fc domain monomer having any of the above-described amino acid substitutions may further include one or more (one, two, three, four, five, six, seven, eight, nine, ten or more) additional mutations (e.g., amino acid deletions, additions, and/or substitutions) relative to the corresponding human wild-type Fc sequence, e.g., a wild-type human IgG sequence. The variant Fc domain monomer can be an IgG subtype (e.g., IgG1, IgG2a, or IgG2b) (e.g., IgG1). A variant Fc domain monomer does not include any portion of an immunoglobulin that is capable of acting as an antigen-recognition region, e.g., a variable domain or a complementarity determining region (CDR). In some embodiments, the variant Fc domain monomer includes between 10 and 20 (e.g., 11, 12, 13, 14, 15, 16, 17, 18, or 19) amino acid residues of the Fab region. In some embodiments, a variant Fc domain monomer (e.g., an IgG heavy chain, such as IgG1) includes a region that extends from any of Asn201 or Glu216 (e.g., Asn201, Val 202, Asn203, His204, Lys 205, Pro206, Ser207, Asn208, Thr209, Lys210, Val211, Asp212, Lys 213, Lys214, Val215, or Glu216), to the carboxyl-terminus of the heavy chain, e.g., at Gly446 or Lys447. C-terminal Lys447 of the Fc region may or may not be present, without affecting the structure or stability of the Fc region. The disclosure specifically contemplates any of SEQ ID NOs: 1-29 and 31-52 that do not include the C-terminal Lys corresponding to Lys447. The variant Fc domain monomer may be expressed including a C-terminal Lys447 which then may be proteolytically cleaved upon expression of the polypeptide (e.g., the variant Fc domain monomer is expressed using a nucleic acid construct encoding the variant Fc domain monomer including a C-terminal lysine residue). The variant Fc domain monomer may also be expressed without including the C-terminal Lys447. The N-terminal Asn201 may be deamidated upon expression of the polypeptide. The N-terminal Asn201 of the variant Fc domain monomer may or may not be present. The presence or absence of the N-terminal Asn201 and/or the C-terminal Lys447 does not affect the structure or stability of the variant Fc domain monomer. The disclosure specifically contemplates any of SEQ ID NOs: 1-29, 31-52, and 56-58 that do not include the N-terminal Asn201 residue. Unless otherwise specified herein, numbering of amino acid residues in variant Fc domain monomer is according to the EU numbering system for antibodies, also called the Kabat EU index, as described, for example, in Kabat et al., Sequences of Proteins of Immunological Interest, 5th Ed. Public Health Service, National Institutes of Health, Bethesda, Md., 1991.

As used herein, the term “variant Fc domain,” refers to a dimer of two variant Fc domain monomers, e.g., that is capable of binding an Fc receptor. In the wild-type Fc domain, the two Fc domain monomers dimerize by the interaction between the two C_{H}3 antibody constant domains, in some embodiments, one or more disulfide bonds form between the hinge domains of the two dimerizing Fc domain monomers.

The terms “Fab” or “fragment antigen-binding,” as used interchangeably herein, refer to a region on an antibody that binds to an antigen. Fab is a term of art and its meaning is known to those of skill in the art. A Fab region is composed of one constant and one variable domain of each of the heavy and light chain. Each heavy chain is comprised of a heavy chain variable region (VH) and a heavy chain constant region (CH). The heavy chain constant region may be comprised of three domains, CH1, CH2, and/or CH3. Each light chain is comprised of a light chain variable region (VL) and a light chain constant region (CL). The VH and VL regions can be further subdivided into regions of hypervariability, termed “complementarity determining regions” (CDRs), interspersed with regions that are more conserved, termed “framework regions” (FRs). In antibodies, the heavy chain (e.g., the VH and CH region) is linked to the Fc domain monomer by way of a hinge. The variant Fc domain monomers described herein may include between 10 and/or 20 residues (e.g., 11, 12, 13, 14, 15, 16, 17, 18, or 19 residues) of the Fab domain and hinge region. In certain embodiments, the N-terminus of the variant Fc domain monomer is any one of amino acid residues 198-205 (corresponding to a residue of the Fab domain). In some embodiments, the N-terminus of the variant Fc domain monomer is amino acid residue 201 (e.g., Asn 201). In certain embodiments, the N-terminus of the variant Fc domain monomer is amino acid residue 202 (e.g., Val 202).

The term “covalently attached” refers to two parts of a conjugate that are linked to each other by a covalent bond formed between two atoms in the two parts of the conjugate.

As used-herein, a “surface exposed amino acid,” or “solvent-exposed amino acid,” such as a surface exposed cysteine or a surface exposed lysine refers to an amino acid that is accessible to the solvent surrounding the protein. A surface exposed amino acid may be a naturally-occurring or an engineered variant (e.g., a substitution or insertion) of the protein. In some embodiments, a surface exposed amino acid is an amino acid that when substituted does not substantially change the three-dimensional structure of the protein.

The term “optionally substituted,” as used herein, refers to having 0, 1, or more substituents, such as 0-25, 0-20, 0-10 or 0-5 substituents. Substituents include, but are not limited to, alkyl, alkenyl, alkynyl, aryl, alkaryl, acyl, heteroaryl, heteroalkyl, heteroalkenyl, heteroalkynyl, heteroalkaryl, halogen, oxo, cyano, nitro, amino, alkamino, hydroxy, alkoxy, alkanoyl, carbonyl, carbamoyl, guanidinyl, ureido, amidinyl, any of the groups or moieties described above, and hetero versions of any of the groups or moieties described above. Substituents include, but are not limited to, F, Cl, methyl, phenyl, benzyl, OR, NR₂, SR, SOR, SO₂R, OCOR, NRCOR, NRCONR₂, NRCOOR, OCONR₂, RCO, COOR, alkyl-OOCR, SO₃R, CONR₂, SO₂NR₂, NRSO₂NR₂, CN, CF₃, OCF₃, SiR₃, and NO₂, wherein each R is, independently, H, alkyl, alkenyl, aryl, heteroalkyl, heteroalkenyl, or heteroaryl, and wherein two of the optional substituents on the same or adjacent atoms can be joined to form a fused, optionally substituted aromatic or nonaromatic, saturated or unsaturated ring which contains 3-8 members, or two of the optional substituents on the same atom can be joined to form an optionally substituted aromatic or nonaromatic, saturated or unsaturated ring which contains 3-8 members.

The term “amino acid,” as used herein, means naturally occurring amino acids and non-naturally occurring amino acids.

The term “naturally occurring amino acids,” as used herein, means amino acids including Ala, Arg, Asn, Asp, Cys, Gln, Glu, Gly, His, lie, Leu, Lys, Met, Phe, Pro, Ser, Thr, Trp, Tyr, and Val.

The term “non-naturally occurring amino acid,” as used herein, means an alpha amino acid that is not naturally produced or found in a mammal. Examples of non-naturally occurring amino acids include D-amino acids; an amino acid having an acetylaminomethyl group attached to a sulfur atom of a cysteine; a pegylated amino acid; the omega amino acids of the formula NH₂(CH₂)ₙCOOH where n is 2-6, neutral nonpolar amino acids, such as sarcosine, t-butyl alanine, t-butyl glycine, N-methyl isoleucine, and norleucine; oxymethionine; phenylglycine; citrulline; methionine sulfoxide; cysteic acid; ornithine; diaminobutyric acid; 3-aminoalanine; 3-hydroxy-D-proline; 2,4-diaminobutyric acid; 2-aminopentanoic acid; 2-aminooctanoic acid, 2-carboxy piperazine; piperazine-2-carboxylic acid, 2-amino-4-phenylbutanoic acid; 3-(2-naphthyl)alanine, and hydroxyproline. Other amino acids are α-aminobutyric acid, α-amino-α-methylbutyrate, aminocyclopropane-carboxylate, aminoisobutyric acid, aminonorbornyl-carboxylate, L-cyclohexylalanine, cyclopentylalanine, L-N-methylleucine, L-N-methylmethionine, L-N-methylnorvaline, L-N-methylphenylalanine, L-N-methylproline, L-N-methylserine, L-N-methyltryptophan, D-ornithine, L-N-methylethylglycine, L-norleucine, α-methyl-aminoisobutyrate, α-methylcyclohexylalanine, D-α-methylalanine, D-α-methylarginine, D-α-methylasparagine, D-α-methylaspartate, D-α-methylcysteine, D-α-methylglutamine, D-α-methylhistidine, D-α-methylisoleucine, D-α-methylleucine, D-α-methyllysine, D-α-methylmethionine, D-α-methylornithine, D-α-methylphenylalanine, D-α-methylproline, D-α-methylserine, D-N-methylserine, D-α-methylthreonine, D-α-methyltryptophan, D-α-methyltyrosine, D-α-methylvaline, D-N-methylalanine, D-N-methylarginine, D-N-methylasparagine, D-N-methylaspartate, D-N-methylcysteine, D-N-methylglutamine, D-N-methylglutamate, D-N-methylhistidine, D-N-methylisoleucine, D-N-methylleucine, D-N-methyllysine, N-methylcyclohexylalanine, D-N-methylornithine, N-methylglycine, N-methylaminoisobutyrate, N-(1-methylpropyl)glycine, N-(2-methylpropyl)glycine, D-N-methyltryptophan, D-N-methyltyrosine, D-N-methylvaline, γ-aminobutyric acid, L-t-butylglycine, L-ethylglycine, L-homophenylalanine, L-α-methylarginine, L-α-methylaspartate, L-α-methylcysteine, L-α-methylglutamine, L-α-methylhistidine, L-α-methylisoleucine, L-α-methylleucine, L-α-methylmethionine, L-α-methylnorvaline, L-α-methylphenylalanine, L-α-methylserine, L-α-methyltryptophan, L-α-methylvaline, N—(N-(2,2-diphenylethyl) carbamylmethylglycine, 1-carboxy-1-(2,2-diphenyl-ethylamino) cyclopropane, 4-hydroxyproline, ornithine, 2-aminobenzoyl (anthraniloyl), D-cyclohexylalanine, 4-phenyl-phenylalanine, L-citrulline, α-cyclohexylglycine, L-1,2,3,4-tetrahydroisoquinoline-3-carboxylic acid, L-thiazolidine-4-carboxylic acid, L-homotyrosine, L-2-furylalanine, L-histidine (3-methyl), N-(3-guanidinopropyl)glycine, O-methyl-L-tyrosine, O-glycan-serine, meta-tyrosine, nor-tyrosine, L-N,N′,N″-trimethyllysine, homolysine, norlysine, N-glycan asparagine, 7-hydroxy-1,2,3,4-tetrahydro-4-fluorophenylalanine, 4-methylphenylalanine, bis-(2-picolyl)amine, pentafluorophenylalanine, indoline-2-carboxylic acid, 2-aminobenzoic acid, 3-amino-2-naphthoic acid, asymmetric dimethylarginine, L-tetrahydroisoquinoline-1-carboxylic acid, D-tetrahydroisoquinoline-1-carboxylic acid, 1-amino-cyclohexane acetic acid, D/L-allylglycine, 4-aminobenzoic acid, 1-amino-cyclobutane carboxylic acid, 2 or 3 or 4-aminocyclohexane carboxylic acid, 1-amino-1-cyclopentane carboxylic acid, 1-aminoindane-1-carboxylic acid, 4-amino-pyrrolidine-2-carboxylic acid, 2-aminotetraline-2-carboxylic acid, azetidine-3-carboxylic acid, 4-benzyl-pyrolidine-2-carboxylic acid, tert-butylglycine, b-(benzothiazolyl-2-yl)-alanine, b-cyclopropyl alanine, 5,5-dimethyl-1,3-thiazolidine-4-carboxylic acid, (2R,4S)4-hydroxypiperidine-2-carboxylic acid, (2S,4S) and (2S,4R)-4-(2-naphthylmethoxy)-pyrolidine-2-carboxylic acid, (2S,4S) and (2S,4R)4-phenoxy-pyrrolidine-2-carboxylic acid, (2R,5S) and (2S,5R)-5-phenyl-pyrrolidine-2-carboxylic acid, (2S,4S)-4-amino-1-benzoyl-pyrrolidine-2-carboxylic acid, t-butylalanine, (2S,5R)-5-phenyl-pyrrolidine-2-carboxylic acid, 1-aminomethyl-cyclohexane-acetic acid, 3,5-bis-(2-amino)ethoxy-benzoic acid, 3,5-diamino-benzoic acid, 2-methylamino-benzoic acid, N-methylanthranylic acid, L-N-methylalanine, L-N-methylarginine, L-N-methylasparagine, L-N-methylaspartic acid, L-N-methylcysteine, L-N-methylglutamine, L-N-methylglutamic acid, L-N-methylhistidine, L-N-methylisoleucine, L-N-methyllysine, L-N-methylnorleucine, L-N-methylornithine, L-N-methylthreonine, L-N-methyltyrosine, L-N-methylvaline, L-N-methyl-t-butylglycine, L-norvaline, α-methyl-γ-aminobutyrate, 4,4′-biphenylalanine, α-methylcylcopentylalanine, α-methyl-α-napthylalanine, α-methylpenicillamine, N-(4-aminobutyl)glycine, N-(2-aminoethyl)glycine, N-(3-aminopropyl)glycine, N-amino-α-methylbutyrate, α-napthylalanine, N-benzylglycine, N-(2-carbamylethyl)glycine, N-(carbamylmethyl)glycine, N-(2-carboxyethyl)glycine, N-(carboxymethyl)glycine, N-cyclobutylglycine, N-cyclodecylglycine, N-cycloheptylglycine, N-cyclohexylglycine, N-cyclodecylglycine, N-cylcododecylglycine, N-cyclooctylglycine, N-cyclopropylglycine, N-cycloundecylglycine, N-(2,2-diphenylethyl)glycine, N-(3,3-diphenylpropyl)glycine, N-(3-guanidinopropyl)glycine, N-(1-hydroxyethyl)glycine, N-(hydroxyethyl))glycine, N-(imidazolylethyl))glycine, N-(3-indolylyethyl)glycine, N-methyl-γ-aminobutyrate, D-N-methylmethionine, N-methylcyclopentylalanine, D-N-methylphenylalanine, D-N-methylproline, D-N-methylthreonine, N-(1-methylethyl)glycine, N-methyl-napthylalanine, N-methylpenicillamine, N-(p-hydroxyphenyl)glycine, N-(thiomethyl)glycine, penicillamine, L-α-methylalanine, L-α-methylasparagine, L-α-methyl-t-butylglycine, L-methylethylglycine, L-α-methylglutamate, L-α-methylhomophenylalanine, N-(2-methylthioethyl)glycine, L-α-methyllysine, L-α-methylnorleucine, L-α-methylornithine, L-α-methylproline, L-α-methylthreonine, L-α-methyltyrosine, L-N-methylhomophenylalanine, N—(N-(3,3-diphenylpropyl) carbamylmethylglycine, L-pyroglutamic acid, D-pyroglutamic acid, O-methyl-L-serine, O-methyl-L-homoserine, 5-hydroxylysine, α-carboxyglutamate, phenylglycine, L-pipecolic acid (homoproline), L-homoleucine, L-lysine (dimethyl), L-2-naphthylalanine, L-dimethyldopa or L-dimethoxy-phenylalanine, L-3-pyridylalanine, L-histidine (benzoyloxymethyl), N-cycloheptylglycine, L-diphenylalanine, O-methyl-L-homotyrosine, L-β-homolysine, O-glycan-threonine, Ortho-tyrosine, L-N,N′-dimethyllysine, L-homoarginine, neotryptophan, 3-benzothienylalanine, isoquinoline-3-carboxylic acid, diaminopropionic acid, homocysteine, 3,4-dimethoxyphenylalanine, 4-chlorophenylalanine, L-1,2,3,4-tetrahydronorharman-3-carboxylic acid, adamantylalanine, symmetrical dimethylarginine, 3-carboxythiomorpholine, D-1,2,3,4-tetrahydronorharman-3-carboxylic acid, 3-aminobenzoic acid, 3-amino-1-carboxymethyl-pyridin-2-one, 1-amino-1-cyclohexane carboxylic acid, 2-aminocyclopentane carboxylic acid, 1-amino-1-cyclopropane carboxylic acid, 2-aminoindane-2-carboxylic acid, 4-amino-tetrahydrothiopyran-4-carboxylic acid, azetidine-2-carboxylic acid, b-(benzothiazol-2-yl)-alanine, neopentylglycine, 2-carboxymethyl piperidine, b-cyclobutyl alanine, allylglycine, diaminopropionic acid, homo-cyclohexyl alanine, (2S,4R)-4-hydroxypiperidine-2-carboxylic acid, octahydroindole-2-carboxylic acid, (2S,4R) and (2S,4R)-4-(2-naphthyl), pyrrolidine-2-carboxylic acid, nipecotic acid, (2S,4R) and (2S,4S)-4-(4-phenylbenzyl) pyrrolidine-2-carboxylic acid, (3S)-1-pyrrolidine-3-carboxylic acid, (2S,4S)-4-tritylmercapto-pyrrolidine-2-carboxylic acid, (2S,4S)-4-mercaptoproline, t-butylglycine, N,N-bis(3-aminopropyl)glycine, 1-amino-cyclohexane-1-carboxylic acid, N-mercaptoethylglycine, and selenocysteine. In some embodiments, amino acid residues may be charged or polar. Charged amino acids include alanine, lysine, aspartic acid, or glutamic acid, or non-naturally occurring analogs thereof. Polar amino acids include glutamine, asparagine, histidine, serine, threonine, tyrosine, methionine, or tryptophan, or non-naturally occurring analogs thereof. It is specifically contemplated that in some embodiments, a terminal amino group in the amino acid may be an amido group or a carbamate group.

The terms “linker,” “L,” and the like as used herein, refer to a covalent linkage or connection between two or more components in a fusion protein or a conjugate (e.g., between a therapeutic peptide agent and a variant Fc domain monomer in order to form a fusion protein, between two therapeutic agents, between a therapeutic agent and a fusion protein, between one or more therapeutic agents and a fusion protein, and between one or more therapeutic agents and a variant Fc domain monomer). In some embodiments, the linker is a bivalent linker, for example a linker connecting a therapeutic peptide agent and a variant Fc domain monomer, a linker connecting a therapeutic agent to a fusion protein, or a linker connecting a therapeutic agent to a variant Fc domain. In some embodiments, a conjugate described herein may contain a linker that has a trivalent structure (e.g., a trivalent linker). A trivalent linker has three arms, in which each arm is covalently linked to a component of the conjugate (e.g., a first arm conjugated to a first therapeutic agent, a second arm conjugated to a therapeutic agent, and a third arm conjugated to the fusion protein or the variant Fc domain monomer). Linkers may be chemical linkers, which are known to one of skill in the art, and are described in detail herein. Chemical linkers can be used to join two small molecules (e.g. to form a dimer), to join a small molecule monomer or small molecule dimer to a polypeptide, or to join two polypeptides to form a fusion protein. Linkers may alternately be peptide linkers. Peptide linkers may also be used to join two small molecules, to join a small molecule monomer or small molecule dimer to a polypeptide, or to join to polypeptides to form a fusion protein.

Molecules that may be used as linkers include at least two functional groups, which may be the same or different, e.g., two carboxylic acid groups, two amine groups, two sulfonic acid groups, a carboxylic acid group and a maleimide group, a carboxylic acid group and an alkyne group, a carboxylic acid group and an amine group, a carboxylic acid group and a sulfonic acid group, an amine group and a maleimide group, an amine group and an alkyne group, or an amine group and a sulfonic acid group. In a bivalent linker, the first functional group may form a covalent linkage with a first component and the second functional group may form a covalent linkage with the second component. In some embodiments, where the linker is a trivalent linker, two arms of a linker may contain two dicarboxylic acids, in which the first carboxylic acid may form a covalent linkage with a first therapeutic agent in the conjugate and the second carboxylic acid may form a covalent linkage with a second therapeutic agent in the conjugate, and the third arm of the linker may for a covalent linkage with the variant Fc domain monomer or fusion protein in the conjugate. Examples of dicarboxylic acids are described further herein. In some embodiments, a molecule containing one or more maleimide groups may be used as a linker, in which the maleimide group may form a carbon-sulfur linkage with a cysteine in a component in the conjugate. In some embodiments, a molecule containing one or more alkyne groups may be used as a linker, in which the alkyne group may form a 1,2,3-triazole linkage with an azide in a component in the conjugate. In some embodiments, a molecule containing one or more azide groups may be used as a linker, in which the azide group may form a 1,2,3-triazole linkage with an alkyne in a component in the conjugate. In some embodiments, a molecule containing one or more bis-sulfone groups may be used as a linker, in which the bis-sulfone group may form a linkage with an amine group a component in the conjugate. In some embodiments, a molecule containing one or more sulfonic acid groups may be used as a linker, in which the sulfonic acid group may form a sulfonamide linkage with a component in the conjugate. In some embodiments, a molecule containing one or more isocyanate groups may be used as a linker, in which the isocyanate group may form a urea linkage with a component in the conjugate. In some embodiments, a molecule containing one or more haloalkyl groups may be used as a linker, in which the haloalkyl group may form a covalent linkage, e.g., C—N and C—O linkages, with a component in the conjugate.

In some embodiments, a linker provides space, rigidity, and/or flexibility between the two or more components. In some embodiments, a linker may be a bond, e.g., a covalent bond. The term “bond” refers to a chemical bond, e.g., an amide bond, a disulfide bond, a C—O bond, a C—N bond, a N—N bond, a C—S bond, or any kind of bond created from a chemical reaction, e.g., chemical conjugation. In some embodiments, a linker includes no more than 250 atoms. In some embodiments, a linker includes no more than 250 non-hydrogen atoms. In some embodiments, the backbone of a linker includes no more than 250 atoms. The “backbone” of a linker refers to the atoms in the linker that together form the shortest path from one part of a conjugate to another part of the conjugate. The atoms in the backbone of the linker are directly involved in linking one part of a conjugate to another part of the conjugate. For examples, hydrogen atoms attached to carbons in the backbone of the linker are not considered as directly involved in linking one part of the conjugate to another part of the conjugate.

In some embodiments, a linker may include a synthetic group derived from, e.g., a synthetic polymer (e.g., a polyethylene glycol (PEG) polymer). In some embodiments, a linker may include one or more amino acid residues, such as D- or L-amino acid residues. In some embodiments, a linker may be a residue of an amino acid sequence (e.g., a 1-25 amino acid, 1-10 amino acid, 1-9 amino acid, 1-8 amino acid, 1-7 amino acid, 1-6 amino acid, 1-5 amino acid, 1-4 amino acid, 1-3 amino acid, 1-2 amino acid, or 1 amino acid sequence). In some embodiments, a linker may include one or more, e.g., 1-100, 1-50, 1-25, 1-10, 1-5, or 1-3, optionally substituted alkylene, optionally substituted heteroalkylene (e.g., a PEG unit), optionally substituted alkenylene, optionally substituted heteroalkenylene, optionally substituted alkynylene, optionally substituted heteroalkynylene, optionally substituted cycloalkylene, optionally substituted heterocycloalkylene, optionally substituted cycloalkenylene, optionally substituted heterocycloalkenylene, optionally substituted cycloalkynylene, optionally substituted heterocycloalkynylene, optionally substituted arylene, optionally substituted heteroarylene (e.g., pyridine), O, S, NRⁱ (Rⁱ is H, optionally substituted alkyl, optionally substituted heteroalkyl, optionally substituted alkenyl, optionally substituted heteroalkenyl, optionally substituted alkynyl, optionally substituted heteroalkynyl, optionally substituted cycloalkyl, optionally substituted heterocycloalkyl, optionally substituted cycloalkenyl, optionally substituted heterocycloalkenyl, optionally substituted cycloalkynyl, optionally substituted heterocycloalkynyl, optionally substituted aryl, or optionally substituted heteroaryl), P, carbonyl, thiocarbonyl, sulfonyl, phosphate, phosphoryl, or imino. For example, a linker may include one or more optionally substituted C1-C20 alkylene, optionally substituted C1-C20 heteroalkylene (e.g., a PEG unit), optionally substituted C2-C20 alkenylene (e.g., C2 alkenylene), optionally substituted C2-C20 heteroalkenylene, optionally substituted C2-C20 alkynylene, optionally substituted C2-C20 heteroalkynylene, optionally substituted C₃-C₂₀cycloalkylene (e.g., cyclopropylene, cyclobutylene), optionally substituted C₂-C₂₀ heterocycloalkylene, optionally substituted C4-C20 cycloalkenylene, optionally substituted C4-C20 heterocycloalkenylene, optionally substituted C8-C20 cycloalkynylene, optionally substituted C8-C20 heterocycloalkynylene, optionally substituted C5-C15 arylene (e.g., C6 arylene), optionally substituted C₃-C₁₅ heteroarylene (e.g., imidazole, pyridine), O, S, NRⁱ (Rⁱ is H, optionally substituted C1-C20 alkyl, optionally substituted C1-C20 heteroalkyl, optionally substituted C2-C20 alkenyl, optionally substituted C2-C20 heteroalkenyl, optionally substituted C2-C20 alkynyl, optionally substituted C2-C20 heteroalkynyl, optionally substituted C₃-C₂₀ cycloalkyl, optionally substituted C₂-C₂₀ heterocycloalkyl, optionally substituted C4-C20 cycloalkenyl, optionally substituted C4-C20 heterocycloalkenyl, optionally substituted C8-C20 cycloalkynyl, optionally substituted C8-C20 heterocycloalkynyl, optionally substituted C5-C15 aryl, or optionally substituted C₃-C₁₅ heteroaryl), P, carbonyl, thiocarbonyl, sulfonyl, phosphate, phosphoryl, or imino.

As used herein, the term “chemical linker” includes any linker described herein that does not include a polypeptide. For example, a chemical linker may include a hydrocarbon chain, which optionally includes one or more heteroatoms (e.g., an optionally substituted alkylene, heteroalkylene, alkenylene, heteroalkenylene, alkynylene, or heteroalkynylene). A chemical linker may include one or more cycloalkyl, heterocycloalkynyl, aryl, or heteroaryl rings within the linker main chain. A chemical linker may include a polyethylene glycol (PEG) polymer, e.g., a PEG₂-PEG₅₀, most preferably PEG₂, PEG₃, PEG₄, PEG₅, PEG₆, PEG₇, PEG₈, PEG₉, or PEG₁₀. A chemical linker may be a bond. As described in greater detail herein (see, e.g., conjugation chemistries), a chemical linker may include at least two functional groups, which may be the same or different, e.g., two carboxylic acid groups, two amine groups, two sulfonic acid groups, a carboxylic acid group and a maleimide group, a carboxylic acid group and an alkyne group, a carboxylic acid group and an amine group, a carboxylic acid group and a sulfonic acid group, an amine group and a maleimide group, an amine group and an alkyne group, or an amine group and a sulfonic acid group. In a bivalent linker, for example, the first functional group may form a covalent linkage with a first component and the second functional group may form a covalent linkage with the second component.

As used interchangeably herein, the terms “peptide linker” or “polypeptide linker” includes any linker than includes two or more amino acid residues. For example, a peptide linker may include 2 or more, 3 or more, 4 or more, 5 or more, 6 or more, 7 or more, 8 or more, 9 or more, 10 or more, 15 or more, 20 or more, 25 or more, 30 or more, 40 or more, or 50 or more amino acid residues, which are joined, for example by peptide bonds. The carboxy terminus of a peptide linker may be covalently conjugated (e.g., by a peptide bond) to a first moiety (e.g., a variant Fc domain monomer or a therapeutic peptide agent) and the amino terminus of the peptide linker may be covalently conjugated (e.g., by a peptide bond) to a second moiety (e.g., a variant Fc domain monomer or a therapeutic peptide agent), thereby conjugating the first moiety and the second moiety and allowing for space and/or flexibility between the first moiety and the second moiety. A peptide linker may be expressed from a polynucleotide construct or chemically synthesized and subsequently chemically conjugated to a first moiety and a second moiety. Alternately, a peptide linker may be expressed in tandem with a first polypeptide (e.g., a variant Fc domain monomer or a therapeutic peptide agent) and a second polypeptide (e.g., a variant Fc domain monomer or a therapeutic peptide agent), thereby joining the first polypeptide and the second polypeptide to form a fusion protein.

As used herein, the term “percent (%) identity” refers to the percentage of amino acid residues of a candidate sequence, e.g., an Fc-IgG, or fragment thereof, that are identical to the amino acid residues of a reference sequence after aligning the sequences and introducing gaps, if necessary, to achieve the maximum percent identity (i.e., gaps can be introduced in one or both of the candidate and reference sequences for optimal alignment and non-homologous sequences can be disregarded for comparison purposes). Alignment for purposes of determining percent identity can be achieved in various ways that are within the skill in the art, for instance, using publicly available computer software such as BLAST, ALIGN, or Megalign (DNASTAR) software. Those skilled in the art can determine appropriate parameters for measuring alignment, including any algorithms needed to achieve maximal alignment over the full length of the sequences being compared. In some embodiments, the percent amino acid sequence identity of a given candidate sequence to, with, or against a given reference sequence (which can alternatively be phrased as a given candidate sequence that has or includes some percent amino acid sequence identity to, with, or against a given reference sequence) is calculated as follows:

100×(fraction of A/B)

where A is the number of amino acid residues scored as identical in the alignment of the candidate sequence and the reference sequence, and where B is the total number of amino acid residues in the reference sequence. In some embodiments where the length of the candidate sequence does not equal to the length of the reference sequence, the percent amino acid sequence identity of the candidate sequence to the reference sequence would not equal to the percent amino acid sequence identity of the reference sequence to the candidate sequence.

Two polynucleotide or polypeptide sequences are said to be “identical” if the sequence of nucleotides or amino acids in the two sequences is the same when aligned for maximum correspondence as described above. Comparisons between two sequences are typically performed by comparing the sequences over a comparison window to identify and compare local regions of sequence similarity. A “comparison window” as used herein, refers to a segment of at least about 15 contiguous positions, about 20 contiguous positions, about 25 contiguous positions, or more (e.g., about 30 to about 75 contiguous positions, or about 40 to about 50 contiguous positions), in which a sequence may be compared to a reference sequence of the same number of contiguous positions after the two sequences are optimally aligned.

As used herein, the term “fusion protein” refers to any conjugate which includes two or more peptides, polypeptides, or proteins, which are covalently linked. The two or more peptides, polypeptides, or proteins may be covalently conjugated by a linker, e.g., any of the linkers described herein, including a chemical linker, a peptide linker, or a bond. For example, a fusion protein may include one or more therapeutic peptide agents and one or more variant Fc domain monomers. The one or more therapeutic peptide agents and one or more variant Fc domain monomers may be encoded by the same polynucleotide sequence (e.g., a single continuous polynucleotide sequence that is operably linked) and expressed as a single polypeptide construct. Alternately, the one or more therapeutic peptide agent and the one or more variant Fc domain monomers may be encoded by separate polynucleotides (e.g., polynucleotide sequences that are not continuous, and can be either on the same vector or separate vectors), expressed as separate polypeptide constructs, and subsequently covalently conjugated by any of the linkers and/or conjugation chemistries described herein. In some instances, the variant Fc domain monomer of the fusion protein may be conjugated to one or more (e.g., 1, 2, 3, 4, 5, 6, 7, 8, 9, 10, or more) small molecule therapeutic agents by way of a linker (e.g., any linker described herein).

As used herein, the term “pharmaceutical composition” refers to a medicinal or pharmaceutical formulation that contains at least one active ingredient (e.g., a conjugate of formula (1), or a fusion protein described herein) as well as one or more excipients and diluents to enable the active ingredient suitable for the method of administration. The pharmaceutical composition of the present disclosure includes pharmaceutically acceptable components that are compatible with a conjugate (e.g., a conjugate of formula (1)) or fusion protein described herein.

As used herein, the term “pharmaceutically acceptable carrier” refers to an excipient or diluent in a pharmaceutical composition. For example, a pharmaceutically acceptable carrier may be a vehicle capable of suspending or dissolving the active conjugate (e.g., a conjugate of formula (1)) or fusion protein described herein. The pharmaceutically acceptable carrier must be compatible with the other ingredients of the formulation and not deleterious to the recipient. In the present disclosure, the pharmaceutically acceptable carrier must provide adequate pharmaceutical stability to a conjugate or fusion protein described herein. The nature of the carrier differs with the mode of administration. For example, for oral administration, a solid carrier is preferred; for intravenous administration, an aqueous solution carrier (e.g., WFI, and/or a buffered solution) is generally used.

The term “pharmaceutically acceptable salt,” as used herein, represents salts of the conjugates described herein (e.g., conjugates of formula (1)) that are, within the scope of sound medical judgment, suitable for use in methods described herein without undue toxicity, irritation, and/or allergic response. Pharmaceutically acceptable salts are well known in the art. For example, pharmaceutically acceptable salts are described in: Pharmaceutical Salts: Properties, Selection, and Use (Eds. P. H. Stahl and C. G. Wermuth), Wiley-VCH, 2008. The salts can be prepared in situ during the final isolation and purification of the conjugates described herein or separately by reacting the free base group with a suitable organic acid.

The term “drug-to-antibody ratio” or “DAR” refers to the average number of small molecule drug moieties (e.g., the average number of small molecule drug monomers or dimers) conjugated to a variant Fc domain monomer or a variant Fc domain described herein. In some embodiments described herein, the DAR is represented by “T” (e.g., in formula (1)). As used herein, each therapeutic agent conjugated to the variant Fc domain corresponds to a DAR value of 1.0 (e.g., a “T” value of 1.0). DAR may also be computed as the average DAR for a population of molecules, such as a population of variant Fc domain conjugates. DAR values may affect the efficacy, potency, pharmacokinetics, or toxicity of the drug.

As used herein, the term “antiviral agent” refers to an agent on any one of the conjugates described herein (e.g., a conjugate of any one of formulas (1)) that exhibits antiviral activity. The antiviral activity exhibited by the antiviral agent can be against any viral infection, e.g., an infection by viral meningitis, herpes simplex virus (HSV) 1, HSV 2, Epstein-Barr virus, varicella-zoster virus, poliovirus, coxsackievirus, West Nile virus, Lacrosse virus, western equine encephalitis, eastern equine encephalitis, Powassan virus, rabies virus, respiratory syncytial virus (RSV), dengue, a beta coronavirus (e.g., COVID-19), zika virus, or an influenza virus. In some examples, the antiviral agent exhibits antiviral activity by interfering with a virus' binding, fusion, and/or entry into a cell.

The term “antibacterial agent,” refers to an agent used in the treatment of a bacterial infection and/or preventing, stabilizing, or inhibiting the growth of bacteria, or killing bacteria. An antibacterial agent may be an agent that prevents the entrance of a bacteria into a subject's cells, tissues, or organs, inhibits the growth of a bacteria in a subject's cells, tissues, or organs, and/or kills a bacteria that is inside a subject's cells, tissues, or organs. In some examples, the antibacterial agent exhibits antibacterial activity by interfering with a bacterium's binding, fusion, and/or entry into a cell. Examples of antibacterial agents are described in detail further herein.

By “viral infection” is meant the pathogenic growth of a virus (e.g., viral meningitis, herpes simplex virus (HSV) 1, HSV 2, Epstein-Barr virus, varicella-zoster virus, poliovirus, coxsackievirus, West Nile virus, Lacrosse virus, western equine encephalitis, eastern equine encephalitis, Powassan virus, rabies virus, respiratory syncytial virus (RSV), dengue, a beta coronavirus (e.g., COVID-19), zika virus, or an influenza virus) in a host organism (e.g., a human subject). A viral infection can be any situation in which the presence of a viral population(s) is damaging to a host body. Thus, a subject is “suffering” from a viral infection when an excessive amount of a viral population is present in or on the subject's body, or when the presence of a viral population(s) is damaging the cells or other tissue of the subject.

By “bacterial infection,” is meant the pathogenic grown of bacteria (e.g., Acinetobacter spp. (Acinetobacter baumanni), Bacteroides distasonis, Bacteroides fragilis, Bacteroides ovatus, Bacteroides thetaiotaomicron, Bacteroides uniformis, Bacteroides vulgatus, Citrobacter freundii, Citrobacter koser, Clostridium clostridioforme, Clostridium perfringens, Enterobacter aerogenes, Enterobacter cloacae, Enterococcus faecalis, Enterococcus spp. (vancomycin susceptible and resistant isolates), Escherichia coli (including ESBL and KPC producing isolates), Eubacterium lentum, Fusobacterium spp., Haemophilus influenzae (including beta-lactamase positive isolates), Haemophilus parainfluenzae, Klebsiella pneumoniae (including ESBL and KPC producing isolates), Klebsiella oxytoca (including ESBL and KPC producing isolates), Legionella pneumophilia Moraxella catarrhalis, Morganella morganii, Mycoplasma spp., Peptostreptococcus spp., Porphyromonas asaccharolytica, Prevotella bivia, Proteus mirabilis, Proteus vulgaris, Providencia rettgeri, Providencia stuartii, Pseudomonas aeruginosa, Serratia marcescens, Streptococcus anginosus, Staphylococcus aureus (methicillin susceptible and resistant isolates), Staphylococcus epidermidis (methicillin susceptible and resistant isolates), Stenotrophomonas maltophilia, Streptococcus agalactiae, Streptococcus constellatus, Streptococcus pneumoniae (penicillin susceptible and resistant isolates), Streptococcus pyogenes) in a host organism (e.g., a human subject). A bacterial infection can be any situation in which the presence of a bacterial population(s) is damaging to a host body. Thus, a subject is “suffering” from a bacterial infection when an excessive amount of a bacterial population(s) is present in or on the subject's body, or when the presence of bacterial population(s) is damaging the cells or other tissue of the subject.

By “fungal infection” is meant the pathogenic grown of a fungus (e.g., Trichophyton species (e.g., T. ajelloi, T. concentricum, T. equinum, T. erinacei, T. flavescens, T. gloriae, T. interdigitale, T. megnini, T. mentagrophytes, T. phaseoliforme, T. rubrum, T. schoenleini, T. simii, T. soudanense, T. terrestre, T. tonsurans, T. vanbreuseghemii, T. verrucosum, T. violaceum, or T. yaoundei), Epidermophyton species (e.g., E. floccosum or E. stockdaleae), Candida species (e.g., C. albicans, C. parapsiliosis, C. krusei, C. tropicalis, C. glabrata, C. parapsilosis, C. lusitaniae, C. kefyr, C. guilliermondii, or C. dubliniensis), Microsporum species (e.g., M. canis, M. gypseum, M. audouini, M. gallinae, M. ferrugineum, M. distortum, M. nanum, M. cookie, or M. vanbreuseghemii), Epicoccum species (e.g., E. nigrum), Aspergillus species (e.g., A. sydowii, A. terreus, A. niger, A. terreus, A. fumigatus, A. flavus, A. clavatus, A. glaucus group, A. nidulans, A. oryzae, A. terreus, A. ustus, or A. versicolor), Paecilomyces species (e.g., P. lilacinus or P. variotii), Fusarium species (e.g., F. oxysporum, F. solani, or F. semitectum), Acremonium species (e.g., A. strictum, A. roseogiseum, A. cucurbitacearum, A. kiliense, A. curvatum, A. comptosporum, Ulocladium chartarum, A. alternatum, or Emercellopsis minima), Chaetomium species (e.g., C. atrobrunneum, C. funicola, C. globosum, or C. strumarium), Phoma species, Scopulariopsis species (e.g., S. brevicaulis, S. candida, S. koningii, S. acremonium, S. flava, S. cinerea, S. trigonospora, S. brumptii, S. chartarum, S. fusca, or S. asperula), Alternaria species (e.g., A. alternate, A. chartarum, A. dianthicola, A. geophilia, A. infectoria, A. stemphyloides, or A. teunissima), and Curvularia species (e.g., C. brachyspora, C. clavata, C. geniculata, C. lunata, C. pallescens, C. senegalensis, or C. verruculosa) in a host organism (e.g., a human subject). A fungal infection can be any situation in which the presence of a fungal population(s) is damaging to a host body. Thus, a subject is “suffering” from a fungal infection when an excessive amount of a fungal population(s) is present in or on the subject's body, or when the presence of fungal population(s) is damaging the cells or other tissue of the subject.

The term “treating” or “to treat,” as used herein, refers to a therapeutic treatment of a disorder (e.g., a respiratory disorder, a hepatic disorder, a central nervous system disorder, a skin disorder, an ocular disorder, vascular disorder, or an infection in a subject. In some embodiments, a therapeutic treatment may slow the progression of the disorder, improve the subject's outcome, and/or eliminate the disorder. In some embodiments, a therapeutic treatment of a disorder in a subject may alleviate or ameliorate of one or more symptoms or conditions associated with the disorder, diminish the extent of the disorder, stabilize (i.e., not worsening) the state of the disorder, prevent the spread of the disorder, and/or delay or slow the progress of the disorder, as compared to the state and/or the condition of the disorder in the absence of the therapeutic treatment.

As used herein, a “combination therapy” or “administered in combination” means that two or more active agents are administered to a subject as part of a defined treatment regimen. The treatment regimen defines the doses and periodicity of administration of each agent such that the effects of the separate agents on the subject overlap. In some embodiments, the delivery of the conjugate and the one or more agents is simultaneous or concurrent and the conjugate and the one or more agents may be co-formulated. In some embodiments, the conjugate and the one or more agents are not co-formulated and are administered in a sequential manner as part of a prescribed regimen. In some embodiments, administration of the conjugate and the one or more agents or treatments in combination is such that the reduction in a symptom, or other parameter related to the viral infection, is greater than what would be observed with one agent or treatment delivered alone or in the absence of the other. The effect of the conjugate and the one or more agents can be partially additive, wholly additive, or greater than additive (e.g., synergistic). Sequential or substantially simultaneous administration of each therapeutic agent can be by any appropriate route including, but not limited to, oral routes, intravenous routes, intramuscular routes, and direct absorption through mucous membrane tissues. The therapeutic agents can be administered by the same route or by different routes. For example, a conjugate or fusion protein described herein may be administered by intravenous injection while a second therapeutic agent of the combination may by another route, e.g., orally.

The term “subject,” as used herein, can be a human, non-human primate, or other mammal, such as but not limited to dog, cat, horse, cow, pig, turkey, goat, fish, monkey, chicken, rat, mouse, and sheep.

The term “therapeutically effective amount,” as used herein, refers to an amount, e.g., pharmaceutical dose, effective in inducing a desired effect in a subject or in treating a subject having a condition or disorder described herein (e.g., a respiratory disorder, a hepatic disorder, a central nervous system disorder, a muscular disorder, a skin disorder, an ocular disorder, a vascular disorder, or an infection (e.g., a viral infection, a fungal infection, or a bacterial infection)). It is also to be understood herein that a “therapeutically effective amount” may be interpreted as an amount giving a desired therapeutic and/or preventative effect, taken in one or more doses or in any dosage or route, and/or taken alone or in combination with other therapeutic agents (e.g., an antiviral agent described herein). For example, in the context of administering a pharmaceutical composition (e.g., a conjugate of formula (1)) or fusion protein described herein) that is used for the treatment of an infection, an effective amount of a conjugate or fusion protein is, for example, an amount sufficient to prevent, slow down, or reverse the progression of the infection (e.g., a viral infection, a fungal infection, or a bacterial infection) as compared to the response obtained without administration of the conjugate or fusion protein.

As used herein, the term “small molecule” refers to a low molecular weight compound (e.g., a compound (e.g., an organic compound) having less than 900 Da, that may regulate a biological process, with a size on the order of 1 nm. In some instances a therapeutic agent is a small molecule therapeutic agent. In some instances, the small molecule agent is between about 300 and about 700 Da (e.g., about 325 Da, about 350 Da, about 375 Da, about 400 Da, about 425 Da, about 450 Da, about 475 Da, about 500 Da, about 525 Da, about 550 Da, about 575 Da, about 600 Da, about 625 Da, about 650 Da, or about 675 Da).

The term “about,” as used herein, indicates a deviation of up to ±5%. For example, about 10% refers to from 9.5% to 10.5%.

Any values provided in a range of values include both the upper and lower bounds, and any values contained within the upper and lower bounds.

Other features and advantages of the conjugates described herein will be apparent from the following Detailed Description and the claims.

### DESCRIPTION OF THE DRAWINGS

FIG. 1 is a graph showing plasma levels of a conjugate including a small molecule conjugated to a variant Fc domain monomer having a C220S/M252Y/S254T/T256E quadruple mutation (SEQ ID NO: 10) (2 mpk IV) compared to a conjugate including an identical small molecule conjugated to an Fc domain having a C220S mutation (SEQ ID NO: 21) (2 mpk IV) in non-human primate PK studies. This study was performed as described in Example 4.

FIG. 2 is a graph showing plasma concentration levels of a small molecule conjugated to a variant Fc domain monomer having a C220S mutation (SEQ ID NO: 21) compared to epithelial lining fluid (ELF) levels of the same conjugate in mice. This study was performed as described in Example 5.

FIG. 3 is a graph showing the plasma levels of a conjugate including a small molecule conjugated to a variant Fc domain monomer having a C220S/M252Y/S254T/T256E quadruple mutation (SEQ ID NO: 10) compared to a conjugate including an identical small molecule conjugated to an Fc domain having a C220S mutation (SEQ ID NO: 21) in mouse PK studies. This study was performed as described in Example 6.

FIG. 4 is a graph showing plasma concentration levels of Fc domain monomers (SEQ ID NOs: 53-55) in mouse PK studies. The graph shows the Fc domain plasma levels increase with increasing molecular weight (SEQ ID NO: 53>SEQ ID NO: 55>SEQ ID NO: 54). This study was performed as described in Example 7.

FIG. 5 is a graph showing average Fc plasma levels of Fc domain monomers (SEQ ID NOs: 53, 56, and 58) in mouse PK studies. This study was performed as described in Example 7.

### DETAILED DESCRIPTION

The present disclosure provides Fc domain monomers, conjugates including an Fc domain monomer, and fusion proteins including an Fc domain monomer, wherein the Fc domain monomer is a mutational variant of a parent Fc polypeptide (e.g., an IgG1 or IgG2 polypeptide). The Fc domain monomers may include one or more mutations that contribute to increased half-life and/or efficacy. The one or more mutations may also minimize aggregation during manufacturing, thereby increasing production and lowering cost. The Fc domain monomers may also be optimized for size (e.g., as measured by kDa or amino acid residues) so as to maximize tissue distribution to a tissue or interest and/or to minimize renal clearance.

In particular, the invention features variant Fc domain monomers including an amino acid mutation at positions 220 (e.g., C220S). The invention features variant Fc domain monomers including amino acid mutations at positions 220, 252, 254, and/or 256 (e.g., C220S/M252Y/S254T/T256E mutations). The invention also includes variant Fc domain monomers including amino acid mutations at positions 220, 309, 311, and/or 434 (e.g., C220S/V309D/Q311H/N434S mutations). The invention also includes conjugates including one or more of the variant Fc domain monomers conjugated to one or more therapeutic agents. The invention further features fusion proteins which include at least one therapeutic peptide agent and at least one variant Fc domain monomer or a conjugate thereof. The variant Fc domain monomer (e.g., of each of two conjugates or two fusion proteins) may dimerize to form a variant Fc domain.

In some instances, the variant Fc domain monomers bind to FcγRs (e.g., FcRn, FcγRI, FcγRIIa, FcγRIIc, FcγRIIIa, and FcγRIIIb) on immune cells, e.g., neutrophils, to activate phagocytosis and effector functions, such as antibody-dependent cell-mediated cytotoxicity (ADCC), thus leading to the engulfment and destruction of infectious agent (e.g., a virus, a fungus, or a bacterium). In other instances, the variant Fc domain monomers further include mutations which decrease or ablate binding to FcγRs (e.g., FcRn, FcγRI, FcγRIIa, FcγRIIc, FcγRIIIa, and FcγRIIIb) on immune cells, e.g., neutrophils and are particularly useful for the delivery of therapeutic agents (e.g., small molecule therapeutic agents and therapeutic peptide agents).

The variant Fc domain monomer and conjugates and fusion proteins thereof exhibit desirable tissue distribution. Such compositions are therefore useful in methods for the treatment of disorders (e.g., respiratory disorders, hepatic disorders, central nervous system disorders, skin disorders, ocular disorders, vascular disorders, inhibition of infection growth, and in methods for the treatment of infections (e.g., viral infections, fungal infections, or bacterial infections).

### I. Variant Fc Domain Monomers and Variant Fc Domains

A variant Fc domain monomer includes a hinge domain, a C_{H}2 antibody constant domain, and a C_{H}3 antibody constant domain. In some embodiments, the variant Fc domain monomer includes a quadruple mutation C220S/M252Y/S254T/T256E. In some embodiments, the variant Fc domain monomers includes a quadruple mutation C220S/V309D/Q311H/N434S. In another embodiment, the variant Fc domain monomer includes a C220S mutation. Amino acid substitutions are relative to a wild-type Fc monomer amino acid sequence, e.g., wild-type human IgG1 or IgG2.

The variant Fc domain monomer can be of immunoglobulin antibody isotype IgG. The variant Fc domain monomer can also be of any immunoglobulin antibody isotype (e.g., IgG1, IgG2a, or IgG2b). The variant Fc domain monomer can be of any immunoglobulin antibody allotype (e.g., IGHG1*01 (i.e., G1m(za)), IGHG1*07 (i.e., G1m(zax)), IGHG1*04 (i.e., G1m(zav)), IGHG1*03 (G1m(f)), IGHG1*08 (i.e., G1m(fa)), IGHG2*01, IGHG2*06, or IGHG2*02) (as described in, for example, in Vidarsson et al. IgG subclasses and allotypes: from structure to effector function. Frontiers in Immunology. 5(520):1-17 (2014)). The variant Fc domain monomer can also be of any species, e.g., human, murine, or mouse. A dimer of variant Fc domain monomers is a variant Fc domain that can bind to an Fc receptor, which is a receptor located on the surface of leukocytes.

In some embodiments, a variant Fc domain monomer includes one or more amino acid substitutions, additions, and/or deletion relative to a variant Fc domain monomer having a sequence of any one of SEQ ID NOs: 1-29, 31-52, or 56-58. In some embodiments, an Asn297 in a variant Fc domain monomer in the conjugates as described herein may be replaced by Ala in order to prevent N-linked glycosylation (see, e.g., SEQ ID NO: 4, where Asn297 to Ala substitution is labeled with (*)).

In some embodiments, the variant Fc domain monomer or variant Fc domain of the invention is an aglycosylated Fc domain monomer or Fc domain (e.g., an Fc domain monomer or and Fc domain that maintains engagement to an Fc receptor (e.g., FcRn). For example, the Fc domain is an aglycosylated IgG1 variant that maintains engagement to an Fc receptor (e.g., an IgG1 having an amino acid substitution at N297 and/or T299 of the glycosylation motif). Exemplary aglycosylated Fc domains and methods for making aglycosylated Fc domains are known in the art, for example, as described in Sazinsky S. L. et al., Aglycosylated immunoglobulin G1 variants productively engage activating Fc receptors, PNAS, 2008, 105(51):20167-20172, which is incorporated herein in its entirety.

C-terminal Lys447 of the Fc region may or may not be present, without affecting the structure or stability of the Fc region. The disclosure specifically contemplates any of SEQ ID NOs: 1-29 and 31-52 that do not include the C-terminal Lys corresponding to Lys447. The N-terminal Asn of the variant Fc domain monomer may or may not be present, without affecting the structure of stability of the variant Fc domain monomer. The disclosure specifically contemplates any of SEQ ID NOs: 1-29, 31-52, and 56-58 that do not include the N-terminal Asn residue.

In some embodiments, a variant Fc domain monomer includes an additional moiety, e.g., a purification peptide (e.g., a hexa-histidine peptide (HHHHHH (SEQ ID NO: 59)), or a signal sequence (e.g., IL2 signal sequence MYRMQLLSCIALSLALVTNS (SEQ ID NO: 60)) attached to the N- or C-terminus of the variant Fc domain monomer. In some embodiments, a variant Fc domain monomer in the conjugate does not contain any type of antibody variable region, e.g., VH, VL, a complementarity determining region (CDR), or a hypervariable region (HVR).

In some embodiments, a variant Fc domain monomer has a sequence that is at least 95% identical (e.g., 97%, 99%, or 99.5% identical) to the sequence of any one of SEQ ID NOs: 1-29, 31-52, and 56-58 shown below. In some embodiments, a variant Fc domain monomer has the sequence of any one of SEQ ID NOs: 1-29, 31-52, and 56-58 shown below.

SEQ ID NO: 1: mature human IgG1 Fc, Cys to Ser substitution (#), YTE triple mutation (bold and underlined), X₁ is Asp or Glu, and X₂ is Leu or Met, N-terminal Fab residues are underlined, hinge residues are italicized

NVNHKPSNTKVDKKVEPKSS(#)DKTHTCPPCPAPELLGGPSVFLFPPK
PKDTLYITREPEVTCVVVDVSHEDPEVKFNWYVDGVEVHNAKTKPREEQ
YNSTYRVVSVLTVLHQDWLNGKEYKCKVSNKALPAPIEKTISKAKGQPR
EPQVYTLPPSRX₁EX₂TKNQVSLTCLVKGFYPSDIAVEWESNGQPENNY
KTTPPVLDSDGSFFLYSKLTVDKSRWQQGNVFSCSVMHEALHNHYTQKS
LSLSPGK

SEQ ID NO: 2: mature human IgG1 Fc, Cys to Ser substitution (#), YTE triple mutation (bold and underlined), allotype G1m(fa) (bold italics), N-terminal Fab residues are underlined, hinge residues are italicized

NVNHKPSNTKVDKKVEPKSS(#)DKTHTCPPCPAP
ELLGGPSVFLFPPKPKDTLYITREPEVTCVVVDVS
HEDPEVKFNWYVDGVEVHNAKTKPREEQYNSTYRV
VSVLTVLHQDWLNGKEYKCKVSNKALPAPIEKTIS
KAKGQPREPQVYTLPPSR [symbol] E [symbol] TKNQVSLTCLVKG
FYPSDIAVEWESNGQPENNYKTTPPVLDSDGSFFL
YSKLTVDKSRWQQGNVFSCSVMHEALHNHYTQKS
LSLSPGK

SEQ ID NO: 3: mature human IgG1 Fc, Cys to Ser substitution (#), YTE triple mutation (bold and underlined), allotype G1m(f) (bold italics), N-terminal Fab residues are underlined, hinge residues are italicized

NVNHKPSNTKVDKKVEPKSS(#)DKTHTCPPCPAP
ELLGGPSVFLFPPKPKDTLYITREPEVTCVVVDVS
HEDPEVKFNWYVDGVEVHNAKTKPREEQYNSTYRV
VSVLTVLHQDWLNGKEYKCKVSNKALPAPIEKTIS
KAKGQPREPQVYTLPPSR [symbol] E [symbol] TKNQVSLTCLVKG
FYPSDIAVEWESNGQPENNYKTTPPVLDSDGSFFL
YSKLTVDKSRWQQGNVFSCSVMHEALHNHYTQKSL
SLSPGK

SEQ ID NO: 4: mature human IgG1 Fc, Cys to Ser substitution (#), YTE triple mutation (bold and underlined), Asn to Ala substitution (*), X₁ is Asp or Glu, and X₂ is Leu or Met, N-terminal Fab residues are underlined, hinge residues are italicized

NVNHKPSNTKVDKKVEPKSS(#)DKTHTCPPCPAP
ELLGGPSVFLFPPKPKDTLYITREPEVTCVVVDVS
HEDPEVKFNWYVDGVEVHNAKTKPREEQYA(*)ST
YRVVSVLTVLHQDWLNGKEYKCKVSNKALPAPIEK
TISKAKGQPREPQVYTLPPSRX₁EX₂TKNQVSLTC
LVKGFYPSDIAVEWESNGQPENNYKTTPPVLDSDG
SFFLYSKLTVDKSRWQQGNVFSCSVMHEALHNHYT
QKSLSLSPGK

SEQ ID NO: 5: mature human IgG1 Fc, Cys to Ser substitution (#), YTE triple mutation (bold and underlined), allotype G1m(fa) (bold italics), Asn to Ala substitution (*), N-terminal Fab residues are underlined, hinge residues are italicized

NVNHKPSNTKVDKKVEPKSS(#)DKTHTCPPCPAP
ELLGGPSVFLFPPKPKDTLYITREPEVTCVVVDVS
HEDPEVKFNWYVDGVEVHNAKTKPREEQYA(*)ST
YRVVSVLTVLHQDWLNGKEYKCKVSNKALPAPIEK
TISKAKGQPREPQVYTLPPSR [symbol] E [symbol] TKNQVSLTCLV
KGFYPSDIAVEWESNGQPENNYKTTPPVLDSDGSF
FLYSKLTVDKSRWQQGNVFSCSVMHEALHNHYTQK
SLSLSPGK

SEQ ID NO: 6: mature human IgG1 Fc, Cys to Ser substitution (#), YTE triple mutation (bold and underlined), allotype G1m(f) (bold italics), Asn to Ala substitution (*), N-terminal Fab residues are underlined, hinge residues are italicized

NVNHKPSNTKVDKKVEPKSS(#)DKTHTCPPCPAP
ELLGGPSVFLFPPKPKDTLYITREPEVTCVVVDVS
HEDPEVKFNWYVDGVEVHNAKTKPREEQYA(*)STY
RVVSVLTVLHQDWLNGKEYKCKVSNKALPAPIEKTI
SKAKGQPREPQVYTLPPSR [symbol] E [symbol] TKNQVSLTCLV
KGFYPSDIAVEWESNGQPENNYKTTPPVLDSDGSFF
LYSKLTVDKSRWQQGNVFSCSVMHEALHNHYTQKSL
SLSPGK

SEQ ID NO: 7: mature human IgG1 Fc, Cys to Ser substitution (#), YTE triple mutation (bold and underlined), X₆ is Asp or Glu, and X₇ is Leu or Met, Z₁ is Asn or absent, Z₂ is Asn or Ala, Z₃ is Lys or absent, N-terminal Fab residues are underlined, hinge residues are italicized

Z₁VNHKPSNTKVDKKVEPKSS(#)DKTHTCPPCPAP
ELLGGPSVFLFPPKPKDTLYITREPEVTCVVVDVS
HEDPEVKFNWYVDGVEVHNAKTKPREEQYZ₂STYRV
VSVLTVLHQDWLNGKEYKCKVSNKALPAPIEKTIS
KAKGQPREPQVYTLPPSRX₆EX₇TKNQVSLTCLVKG
FYPSDIAVEWESNGQPENNYKTTPPVLDSDGSFFLY
SKLTVDKSRWQQGNVFSCSVMHEALHNHYTQKSLSL
SPGZ₃

SEQ ID NO: 8: mature human IgG1 Fc, Cys to Ser substitution (#), YTE triple mutation (bold and underlined), allotype G1m(fa) (bold italics), N-terminal Fab residues are underlined, hinge residues are italicized

VNHKPSNTKVDKKVEPKSS(#)DKTHTCPPCPAP
ELLGGPSVFLFPPKPKDTLYITREPEVTCVVVDVS
HEDPEVKFNWYVDGVEVHNAKTKPREEQYNSTYRV
VSVLTVLHQDWLNGKEYKCKVSNKALPAPIEKTIS
KAKGQPREPQVYTLPPSR [symbol] E [symbol] TKNQVSLTCLV
KGFYPSDIAVEWESNGQPENNYKTTPPVLDSDGSF
FLYSKLTVDKSRWQQGNVFSCSVMHEALHNHYTQK
SLSLSPGK

SEQ ID NO: 9: mature human IgG1 Fc, Cys to Ser substitution (#), YTE triple mutation (bold and underlined), allotype G1m(f) (bold italics), N-terminal Fab residues are underlined, hinge residues are italicized

VNHKPSNTKVDKKVEPKSS(#)DKTHTCPPCPAP
ELLGGPSVFLFPPKPKDTLYITREPEVTCVVVDVS
HEDPEVKFNWYVDGVEVHNAKTKPREEQYNSTYRV
VSVLTVLHQDWLNGKEYKCKVSNKALPAPIEKTIS
KAKGQPREPQVYTLPPSR [symbol] E [symbol] TKNQVSLTCLV
KGFYPSDIAVEWESNGQPENNYKTTPPVLDSDGSF
FLYSKLTVDKSRWQQGNVFSCSVMHEALHNHYTQK
SLSLSPGK

SEQ ID NO: 10: mature human IgG1 Fc, Cys to Ser substitution (#), YTE triple mutation (bold and underlined), allotype G1m(fa) (bold italics), N-terminal Fab residues are underlined, hinge residues are italicized

NVNHKPSNTKVDKKVEPKSS(#)DKTHTCPPCPAP
ELLGGPSVFLFPPKPKDTLYITREPEVTCVVVDVS
HEDPEVKFNWYVDGVEVHNAKTKPREEQYNSTYRV
VSVLTVLHQDWLNGKEYKCKVSNKALPAPIEKTIS
KAKGQPREPQVYTLPPSR [symbol] E [symbol] TKNQVSLTCLV
KGFYPSDIAVEWESNGQPENNYKTTPPVLDSDGSF
FLYSKLTVDKSRWQQGNVFSCSVMHEALHNHYTQK
SLSLSPG

SEQ ID NO: 11: mature human IgG1 Fc, Cys to Ser substitution (#), YTE triple mutation (bold and underlined), allotype G1m(f) (bold italics), N-terminal Fab residues are underlined, hinge residues are italicized

NVNHKPSNTKVDKKVEPKSS(#)DKTHTCPPCPAP
ELLGGPSVFLFPPKPKDTLYITREPEVTCVVVDVS
HEDPEVKFNWYVDGVEVHNAKTKPREEQYNSTYRV
VSVLTVLHQDWLNGKEYKCKVSNKALPAPIEKTIS
KAKGQPREPQVYTLPPSR [symbol] E [symbol] TKNQVSLTCLV
KGFYPSDIAVEWESNGQPENNYKTTPPVLDSDGSF
FLYSKLTVDKSRWQQGNVFSCSVMHEALHNHYTQK
SLSLSPG

SEQ ID NO: 12: mature human IgG1 Fc, Cys to Ser substitution (#), YTE triple mutation (bold and underlined), allotype G1m(fa) (bold italics), N-terminal Fab residues are underlined, hinge residues are italicized

VNHKPSNTKVDKKVEPKSS(#)DKTHTCPPCPAP
ELLGGPSVFLFPPKPKDTLYITREPEVTCVVVDVS
HEDPEVKFNWYVDGVEVHNAKTKPREEQYNSTYRV
VSVLTVLHQDWLNGKEYKCKVSNKALPAPIEKTIS
KAKGQPREPQVYTLPPSR [symbol] E [symbol] TKNQVSLTCLV
KGFYPSDIAVEWESNGQPENNYKTTPPVLDSDGSF
FLYSKLTVDKSRWQQGNVFSCSVMHEALHNHYTQK
SLSLSPG

SEQ ID NO: 13: mature human IgG1 Fc, Cys to Ser substitution (#), YTE triple mutation (bold and underlined), allotype G1m(f) (bold italics), N-terminal Fab residues are underlined, hinge residues are italicized

VNHKPSNTKVDKKVEPKSS(#)DKTHTCPPCPAP
ELLGGPSVFLFPPKPKDTLYITREYITREPEVTCV
VVDVSHEDPEVKFNWYVDGVEVHNAKTKPREEQYN
STYRVVSVLTVLHQDWLNGKEYKCKVSNKALPAPI
EKTISKAKGQPREPQVYTLPPSR [symbol] E [symbol] TKNQVS
LTCLVKGFYPSDIAVEWESNGQPENNYKTTPPVLD
SDGSFFLYSKLTVDKSRWQQGNVFSCSVMHEALHN
HYTQKSLSLSPG

SEQ ID NO: 14: mature human IgG1 Fc, Cys to Ser substitution (#), YTE triple mutation (bold and underlined), allotype G1m(fa) (bold italics), Asn to Ala substitution (*), N-terminal Fab residues are underlined, hinge residues are italicized

VNHKPSNTKVDKKVEPKSS(#)DKTHTCPPCPAP
ELLGGPSVFLFPPKPKDTLYITREPEVTCVVVDVS
HEDPEVKFNWYVDGVEVHNAKTKPREEQYA(*)ST
YRVVSVLTVLHQDWLNGKEYKCKVSNKALPAPIEK
TISKAKGQPREPQVYTLPPSR [symbol] E [symbol] TKNQVSLT
CLVKGFYPSDIAVEWESNGQPENNYKTTPPVLDSD
GSFFLYSKLTVDKSRWQQGNVFSCSVMHEALHNHY
TQKSLSLSPGK

SEQ ID NO: 15: mature human IgG1 Fc, Cys to Ser substitution (#), YTE triple mutation (bold and underlined), allotype G1m(f) (bold italics), Asn to Ala substitution (*), N-terminal Fab residues are underlined, hinge residues are italicized

VNHKPSNTKVDKKVEPKSS(#)DKTHTCPPCPAP
ELLGGPSVFLFPPKPKDTLYITREPEVTCVVVDVS
HEDPEVKFNWYVDGVEVHNAKTKPREEQYA(*)ST
YRVVSVLTVLHQDWLNGKEYKCKVSNKALPAPIEK
TISKAKGQPREPQVYTLPPSR [symbol] E [symbol] TKNQVSLT
CLVKGFYPSDIAVEWESNGQPENNYKTTPPVLDSD
GSFFLYSKLTVDKSRWQQGNVFSCSVMHEALHNHY
TQKSLSLSPGK

SEQ ID NO: 16: mature human IgG1 Fc, Cys to Ser substitution (#), YTE triple mutation (bold and underlined), allotype G1m(fa) (bold italics), Asn to Ala substitution (*), N-terminal Fab residues are underlined, hinge residues are italicized

NVNHKPSNTKVDKKVEPKSS(#)DKTHTCPPCPAP
ELLGGPSVFLFPPKPKDTLYITREPEVTCVVVDVS
HEDPEVKFNWYVDGVEVHNAKTKPREEQYA(*)ST
YRVVSVLTVLHQDWLNGKEYKCKVSNKALPAPIEK
TISKAKGQPREPQVYTLPPSR [symbol] E [symbol] TKNQVSLT
CLVKGFYPSDIAVEWESNGQPENNYKTTPPVLDSD
GSFFLYSKLTVDKSRWQQGNVFSCSVMHEALHNHY
TQKSLSLSPG

SEQ ID NO: 17: mature human IgG1 Fc, Cys to Ser substitution (#), YTE triple mutation (bold and underlined), allotype G1m(f) (bold italics), Asn to Ala substitution (*), N-terminal Fab residues are underlined, hinge residues are italicized

NVNHKPSNTKVDKKVEPKSS(#)DKTHTCPPCPAPELLGGPSVFLFPPKPKDTLYITREPEVTCVVV
DVSHEDPEVKFNWYVDGVEVHNAKTKPREEQYA(*)STYRVVSVLTVLHQDWLNGKEYKCKVSNKAL
PAPIEKTISKAKGQPREPQVYTLPPSR [symbol] E [symbol] TKNQVSLTCLVKGFYPSDIAVEWESNGQPENNYKTT
PPVLDSDGSFFLYSKLTVDKSRWQQGNVFSCSVMHEALHNHYTQKSLSLSPG

SEQ ID NO: 18: mature human IgG1 Fc, Cys to Ser substitution (#), YTE triple mutation (bold and underlined), allotype G1m(fa) (bold italics), Asn to Ala substitution (*), N-terminal Fab residues are underlined, hinge residues are italicized

VNHKPSNTKVDKKVEPKSS(#)DKTHTCPPCPAPELLGGPSVFLFPPKPKDTLYITREPEVTCVVVD
VSHEDPEVKFNWYVDGVEVHNAKTKPREEQYA(*)STYRVVSVLTVLHQDWLNGKEYKCKVSNKALP
APIEKTISKAKGQPREPQVYTLPPSR [symbol] E [symbol] TKNQVSLTCLVKGFYPSDIAVEWESNGQPENNYKTTP
PVLDSDGSFFLYSKLTVDKSRWQQGNVFSCSVMHEALHNHYTQKSLSLSPG

SEQ ID NO: 19: mature human IgG1 Fc, Cys to Ser substitution (#), YTE triple mutation (bold and underlined), allotype G1m(f) (bold italics), Asn to Ala substitution (*), N-terminal Fab residues are underlined, hinge residues are italicized

VNHKPSNTKVDKKVEPKSS(#)DKTHTCPPCPAPELLGGPSVFLFPPKPKDTLYITREPEVTCVVVD
VSHEDPEVKFNWYVDGVEVHNAKTKPREEQYA(*)STYRVVSVLTVLHQDWLNGKEYKCKVSNKALP
APIEKTISKAKGQPREPQVYTLPPSR [symbol] E [symbol] TKNQVSLTCLVKGFYPSDIAVEWESNGQPENNYKTTP
PVLDSDGSFFLYSKLTVDKSRWQQGNVFSCSVMHEALHNHYTQKSLSLSPG

SEQ ID NO: 20: mature human IgG1 Fc, Cys to Ser substitution (#), X₄ is Asp or Glu, X₅ is Leu or Met; Z₁ is Asn or absent, Z₃ is Lys or absent, N-terminal Fab residues are underlined, hinge residues are italicized

Z₁VNHKPSNTKVDKKVEPKSS(#)DKTHTCPPCPAPELLGGPSVFLFPPKPKDTLMISRTPEVTCVV
VDVSHEDPEVKFNWYVDGVEVHNAKTKPREEQYNSTYRVVSVLTVLHQDWLNGKEYKCKVSNKALPA
PIEKTISKAKGQPREPQVYTLPPSRX₄EX₅TKNQVSLTCLVKGFYPSDIAVEWESNGQPENNYKTTP
PVLDSDGSFFLYSKLTVDKSRWQQGNVFSCSVMHEALHNHYTQKSLSLSPGZ₃

SEQ ID NO: 21: mature human IgG1 Fc, Cys to Ser substitution (#), allotype G1m(fa) (bold italics), N-terminal Fab residues are underlined, hinge residues are italicized

NVNHKPSNTKVDKKVEPKSS(#)DKTHTCPPCPAPELLGGPSVFLFPPKPKDTLMISRTPEVTCVVV
DVSHEDPEVKFNWYVDGVEVHNAKTKPREEQYNSTYRVVSVLTVLHQDWLNGKEYKCKVSNKALPAP
IEKTISKAKGQPREPQVYTLPPSR [symbol] E [symbol] TKNQVSLTCLVKGFYPSDIAVEWESNGQPENNYKTTP
PVLDSDGSFFLYSKLTVDKSRWQQGNVFSCSVMHEALHNHYTQKSLSLSPGK

SEQ ID NO: 22: mature human IgG1 Fc, Cys to Ser substitution (#), allotype G1 m(f) (bold italics), N-terminal Fab residues are underlined, hinge residues are italicized

NVNHKPSNTKVDKKVEPKSS(#)DKTHTCPPCPAPELLGGPSVFLFPPKPKDTLMISRTPEVTCVVV
DVSHEDPEVKFNWYVDGVEVHNAKTKPREEQYNSTYRVVSVLTVLHQDWLNGKEYKCKVSNKALPAP
IEKTISKAKGQPREPQVYTLPPSR [symbol] E [symbol] TKNQVSLTCLVKGFYPSDIAVEWESNGQPENNYKTTP
PVLDSDGSFFLYSKLTVDKSRWQQGNVFSCSVMHEALHNHYTQKSLSLSPGK

SEQ ID NO: 23: mature human IgG1 Fc, Cys to Ser substitution (#), X₁ is Met or Tyr, X₂ is Ser or Thr, X₃ is Thr or Glu, X₆ is Asp or Glu, X₇ is Leu or Met, Z₁ is Asn or absent, Z₂ is Asn or Ala, and Z₃ is Lys or absent, N-terminal Fab residues are underlined, hinge residues are italicized

Z₁VNHKPSNTKVDKKVEPKSS(#)DKTHTCPPCPAPELLGGPSVFLFPPKPKDTLX₁IX₂RX₃PEVTCV
VVDVSHEDPEVKFNWYVDGVEVHNAKTKPREEQYZ₂STYRVVSVLTVLHQDWLNGKEYKCKVSNKALP
APIEKTISKAKGQPREPQVYTLPPSRX₆EX₇TKNQVSLTCLVKGFYPSDIAVEWESNGQPENNYKTTP
PVLDSDGSFFLYSKLTVDKSRWQQGNVFSCSVMHEALHNHYTQKSLSLSPGZ₃

SEQ ID NO: 24: mature human IgG1 Fc, Cys to Ser substitution (#), X₁ is Met or Tyr, X₂ is Ser or Thr, X₃ is Thr or Glu, X₆ is Asp or Glu, X₇ is Leu or Met, and Z₂ is Asn or Ala, N-terminal Fab residues are underlined, hinge residues are italicized

VNHKPSNTKVDKKVEPKSS(#)DKTHTCPPCPAPELLGGPSVFLFPPKPKDTLX₁IX₂RX₃PEVTCVV
VDVSHEDPEVKFNWYVDGVEVHNAKTKPREEQYZ₂STYRVVSVLTVLHQDWLNGKEYKCKVSNKALP
APIEKTISKAKGQPREPQVYTLPPSRX₆EX₇TKNQVSLTCLVKGFYPSDIAVEWESNGQPENNYKTT
PPVLDSDGSFFLYSKLTVDKSRWQQGNVFSCSVMHEALHNHYTQKSLSLSPGK

SEQ ID NO: 25: mature human IgG1 Fc, Cys to Ser substitution (#), X₁ is Met or Tyr, X₂ is Ser or Thr, X₃ is Thr or Glu, X₄ is Asp or Glu, X₅ is Leu or Met, and Z₂ is Asn or Ala, N-terminal Fab residues are underlined, hinge residues are italicized

VNHKPSNTKVDKKVEPKSS(#)DKTHTCPPCPAPELLGGPSVFLFPPKPKDTLX₁IX₂RX₃PEVTCVV
VDVSHEDPEVKFNWYVDGVEVHNAKTKPREEQYZ₂STYRVVSVLTVLHQDWLNGKEYKCKVSNKALP
APIEKTISKAKGQPREPQVYTLPPSR [symbol] E [symbol] TKNQVSLTCLVKGFYPSDIAVEWESNGQPENNYKT
TPPVLDSDGSFFLYSKLTVDKSRWQQGNVFSCSVMHEALHNHYTQKSLSLSPGK

SEQ ID NO: 26: mature human IgG1 Fc, Cys to Ser substitution (#), X₁ is Met or Tyr, X₂ is Ser or Thr, X₃ is Thr or Glu, X₄ is Asp or Glu, X₅ is Leu or Met, and Z₂ is Asn or Ala, N-terminal Fab residues are underlined, hinge residues are italicized

VNHKPSNTKVDKKVEPKSS(#)DKTHTCPPCPAPELLGGPSVFLFPPKPKDTLX₁IX₂RX₃PEVTCVV
VDVSHEDPEVKFNWYVDGVEVHNAKTKPREEQYZ₂STYRVVSVLTVLHQDWLNGKEYKCKVSNKALP
APIEKTISKAKGQPREPQVYTLPPSR [symbol] E [symbol] TKNQVSLTCLVKGFYPSDIAVEWESNGQPENNYKT
TPPVLDSDGSFFLYSKLTVDKSRWQQGNVFSCSVMHEALHNHYTQKSLSLSPGK

SEQ ID NO: 27: mature human IgG1 Fc, Cys to Ser substitution (#), X₁ is Met or Tyr, X₂ is Ser or Thr, X₃ is Thr or Glu, X₆ is Asp or Glu, X₇ is Leu or Met, and Z₂ is Asn or Ala, N-terminal Fab residues are underlined, hinge residues are italicized

VNHKPSNTKVDKKVEPKSS(#)DKTHTCPPCPAPELLGGPSVFLFPPKPKDTLX₁IX₂RX₃PEVTCVV
VDVSHEDPEVKFNWYVDGVEVHNAKTKPREEQYZ₂STYRVVSVLTVLHQDWLNGKEYKCKVSNKALP
APIEKTISKAKGQPREPQVYTLPPSRX₆EX₇TKNQVSLTCLVKGFYPSDIAVEWESNGQPENNYKTT
PPVLDSDGSFFLYSKLTVDKSRWQQGNVFSCSVMHEALHNHYTQKSLSLSPG

SEQ ID NO: 28: mature human IgG1 Fc, Cys to Ser substitution (#), X₁ is Met or Tyr, X₂ is Ser or Thr, X₃ is Thr or Glu, and Z₂ is Asn or Ala, N-terminal Fab residues are underlined, hinge residues are italicized

VNHKPSNTKVDKKVEPKSS(#)DKTHTCPPCPAPELLGGPSVFLFPPKPKDTLX₁IX₂RX₃PEVTCVV
VDVSHEDPEVKFNWYVDGVEVHNAKTKPREEQYZ₂STYRVVSVLTVLHQDWLNGKEYKCKVSNKALP
APIEKTISKAKGQPREPQVYTLPPSR [symbol] E [symbol] TKNQVSLTCLVKGFYPSDIAVEWESNGQPENNYKT
TPPVLDSDGSFFLYSKLTVDKSRWQQGNVFSCSVMHEALHNHYTQKSLSLSPG

SEQ ID NO: 29: mature human IgG1 Fc, Cys to Ser substitution (#), X₁ is Met or Tyr, X₂ is Ser or Thr, X₃ is Thr or Glu, and Z₂ is Asn or Ala, N-terminal Fab residues are underlined, hinge residues are italicized

VNHKPSNTKVDKKVEPKSS(#)DKTHTCPPCPAPELLGGPSVFLFPPKPKDTLX₁IX₂RX₃PEVTCVV
VDVSHEDPEVKFNWYVDGVEVHNAKTKPREEQYZ₂STYRVVSVLTVLHQDWLNGKEYKCKVSNKALP
APIEKTISKAKGQPREPQVYTLPPSR [symbol] E [symbol] TKNQVSLTCLVKGFYPSDIAVEWESNGQPENNYKT
TPPVLDSDGSFFLYSKLTVDKSRWQQGNVFSCSVMHEALHNHYTQKSLSLSPG

SEQ ID NO: 30: mature human IgG1 Fc with mouse heavy chain MIgG Vh signal sequence (bold), Cys to Ser substitution (#), allotype G1m(fa) (bold italics), N-terminal Fab residues are underlined, hinge residues are italicized

MGWSCIILFLVATATGVHSNVNHKPSNTKVDKKVEPKSS(#)DKTHTCPPCPAPELLGGPSVFLFPP
KPKDTLMISRTPEVTCVVVDVSHEDPEVKFNWYVDGVEVHNAKTKPREEQYNSTYRVVSVLTVLHQD
WLNGKEYKCKVSNKALPAPIEKTISKAKGQPREPQVYTLPPSR [symbol] E [symbol] TKNQVSLTCLVKGFYPSD
IAVEWESNGQPENNYKTTPPVLDSDGSFFLYSKLTVDKSRWQQGNVFSCSVMHEALHNHYTQKSLSL
SPGK

SEQ ID NO: 31: mature human Fc IgG1, Z₁ is Asn or absent, Z₂ is Lys or absent, J₁ is Cys or Ser, and wherein X₁ is Met or Tyr, X₂ is Ser or Thr, X₃ is Thr or Glu, Z₂ is Asn or Ala, X₄ is Leu or Asp, X₅ is Gln or His, X₆ is Asp or Glu, and X₇ is Leu or Met, X₈ is Met or Leu, and X₉ is Asn or Ser, N-terminal Fab residues are underlined, hinge residues are italicized

Z₁VNHKPSNTKVDKKVEPKS [symbol] ₁DKTHTCPPCPAPELLGGPSVFLFPPKPKDTLX₁IX₂RX₃PEVTCVV
VDVSHEDPEVKFNWYVDGVEVHNAKTKPREEQYZ₂STYRVVSVLTVX₄HX₅DWLNGKEYKCKVSNKAL
PAPIEKTISKAKGQPREPQVYTLPPSRX₆EX₇TKNQVSLTCLVKGFYPSDIAVEWESNGQPENNYKT
TPPVLDSDGSFFLYSKLTVDKSRWQQGNVFSCSVX₈HEALHX₉HYTQKSLSLSPGZ₃

SEQ ID NO: 32: mature human Fc IgG1, Cys to Ser substitution (#), Z₁ is Asn or absent, Z₃ is Lys or absent, and wherein Z₂ is Asn or Ala, X₄ is Leu or Asp, X₅ is Gln or His, X₆ is Asp or Glu, X₇ is Leu or Met, X₈ is Met or Leu, and X₉ is Asn or Ser, N-terminal Fab residues are underlined, hinge residues are italicized

Z₁VNHKPSNTKVDKKVEPKSS(#)DKTHTCPPCPAPELLGGPSVFLFPPK
PKDTLMISRTPEVTCVVVDVSHEDPEVKFNWYVDGVEVHNAKTKPREEQY
Z₂STYRVVSVLTVX₄HX₅DWLNGKEYKCKVSNKALPAPIEKTISKAKGQPR
EPQVYTLPPSRX₆EX₇TKNQVSLTCLVKGFYPSDIAVEWESNGQPENNYKT
TPPVLDSDGSFFLYSKLTVDKSRWQQGNVFSCSVX₈HEALHX₉HYTQKSLS
LSPGZ₃

SEQ ID NO: 33: mature human Fc IgG1, Cys to Ser substitution (#), DHS triple mutation (bold and underlined), Z₁ is Asn or absent, Z₃ is Lys or absent, and wherein Z₂ is Asn or Ala, X₆ is Asp or Glu, and X₇ is Leu or Met, X₈ is Met or Leu, and X₉ is Asn or Ser, N-terminal Fab residues are underlined, hinge residues are italicized

Z₁VNHKPSNTKVDKKVEPKSS(#)DKTHTCPPCPAPELLGGPSVFLFPPK
PKDTLMISRTPEVTCVVVDVSHEDPEVKFNWYVDGVEVHNAKTKPREEQY
Z₂STYRVVSVLTVDHHDWLNGKEYKCKVSNKALPAPIEKTISKAKGQPRE
PQVYTLPPSRX₆EX₇TKNQVSLTCLVKGFYPSDIAVEWESNGQPENNYKTT
PPVLDSDGSFFLYSKLTVDKSRWQQGNVFSCSVX₈HEALHX₉HYTQKSLSL
SPGZ₃

SEQ ID NO: 34: mature human Fc IgG1, Cys to Ser substitution (#), DHS triple mutation (bold and underlined), wherein Z₂ is Asn or Ala, X₆ is Asp or Glu, and X₇ is Leu or Met, N-terminal Fab residues are underlined, hinge residues are italicized

NVNHKPSNTKVDKKVEPKSS(#)DKTHTCPPCPAPELLGGPSVFLFPPK
PKDTLMISRTPEVTCVVVDVSHEDPEVKFNWYVDGVEVHNAKTKPREEQ
YZ₂STYRVVSVLTVDHHDWLNGKEYKCKVSNKALPAPIEKTISKAKGQPR
EPQVYTLPPSRX₆EX₇TKNQVSLTCLVKGFYPSDIAVEWESNGQPENNYK
TTPPVLDSDGSFFLYSKLTVDKSRWQQGNVFSCSVMHEALHSHYTQKSL
SLSPGK

SEQ ID NO: 35: mature human Fc IgG1, Cys to Ser substitution (#), DHS triple mutation (bold and underlined), wherein X₆ is Asp or Glu and X₇ is Leu or Met, N-terminal Fab residues are underlined, hinge residues are italicized

NVNHKPSNTKVDKKVEPKSS(#)DKTHTCPPCPAPELLGGPSVFLFPPK
PKDTLMISRTPEVTCVVVDVSHEDPEVKFNWYVDGVEVHNAKTKPREEQ
YNSTYRVVSVLTVDHHDWLNGKEYKCKVSNKALPAPIEKTISKAKGQPR
EPQVYTLPPSRX₆EX₇TKNQVSLTCLVKGFYPSDIAVEWESNGQPENNYK
TTPPVLDSDGSFFLYSKLTVDKSRWQQGNVFSCSVMHEALHSHYTQKSL
SLSPGK

SEQ ID NO: 36: mature human Fc IgG1, Cys to Ser substitution (#), DHS triple mutation (bold and underlined), allotype G1m(fa) (bold italics), N-terminal Fab residues are underlined, hinge residues are italicized

NVNHKPSNTKVDKKVEPKSS(#)DKTHTCPPCPAPELLGGPSVFLFPPK
PKDTLMISRTPEVTCVVVDVSHEDPEVKFNWYVDGVEVHNAKTKPREEQ
YNSTYRVVSVLTVDHHDWLNGKEYKCKVSNKALPAPIEKTISKAKGQPR
EPQVYTLPPSR [symbol] E [symbol] TKNQVSLTCLVKGFYPSDIAVEWESNGQPEN
NYKTTPPVLDSDGSFFLYSKLTVDKSRWQQGNVFSCSVMHEALHSHYTQ
KSLSLSPGK

SEQ ID NO: 37: mature human Fc IgG1, Cys to Ser substitution (#), DHS triple mutation (bold and underlined), allotype G1m(f) (bold italics), N-terminal Fab residues are underlined, hinge residues are italicized

NVNHKPSNTKVDKKVEPKSS(#)DKTHTCPPCPAPELLGGPSVFLFPP
KPKDTLMISRTPEVTCVVVDVSHEDPEVKFNWYVDGVEVHNAKTKPRE
EQYNSTYRVVSVLTVDHHDWLNGKEYKCKVSNKALPAPIEKTISKAKG
QPREPQVYTLPPSR [symbol] E [symbol] TKNQVSLTCLVKGFYPSDIAVEWESNG
QPENNYKTTPPVLDSDGSFFLYSKLTVDKSRWQQGNVFSCSVMHEALH
SHYTQKSLSLSPGK

SEQ ID NO: 38: mature human Fc IgG1, Cys to Ser substitution (#), DHS triple mutation (bold and underlined), allotype G1m(fa) (bold italics), N-terminal Fab residues are underlined, hinge residues are italicized

VNHKPSNTKVDKKVEPKSS(#)DKTHTCPPCPAPELLGGPSVFLFPP
KPKDTLMISRTPEVTCVVVDVSHEDPEVKFNWYVDGVEVHNAKTKPR
EEQYNSTYRVVSVLTVDHHDWLNGKEYKCKVSNKALPAPIEKTISKA
KGQPREPQV YTLPPSR [symbol] E [symbol] TKNQVSLTCLVKGFYPSDIAVEW
ESNGQPENNYKTTPPVLDSDGSFFLYSKLTVDKSRWQQGNVFSCSVM
HEALHSHYTQKSLSLSPGK

SEQ ID NO: 39: mature human Fc IgG1, Cys to Ser substitution (#), DHS triple mutation (bold and underlined), allotype G1m(f) (bold italics), N-terminal Fab residues are underlined, hinge residues are italicized

VNHKPSNTKVDKKVEPKSS(#)DKTHTCPPCPAPELLGGPSVFLF
PPKPKDTLMISRTPEVTCVVVDVSHEDPEVKFNWYVDGVEVHNAK
TKPREEQYNSTYRVVSVLTVDHHDWLNGKEYKCKVSNKALPAPIE
KTISKAKGQPREPQVYTLPPSR [symbol] E [symbol] TKNQVSLTCLVKGFYP
SDIAVEWESNGQPENNYKTTPPVLDSDGSFFLYSKLTVDKSRWQQ
GNVFSCSVMHEALHSHYTQKSLSLSPGK

SEQ ID NO: 40: mature human Fc IgG1, Cys to Ser substitution (#), DHS triple mutation (bold and underlined), allotype G1m(fa) (bold italics), N-terminal Fab residues are underlined, hinge residues are italicized

NVNHKPSNTKVDKKVEPKSS(#)DKTHTCPPCPAPELLGGPSVF
LFPPKPKDTLMISRTPEVTCVVVDVSHEDPEVKFNWYVDGVEVH
NAKTKPREEQYNSTYRVVSVLTVDHHDWLNGKEYKCKVSNKALP
APIEKTISKAKGQPREPQVYTLPPSR [symbol] E [symbol] TKNQVSLTCLV
KGFYPSDIAVEWESNGQPENNYKTTPPVLDSDGSFFLYSKLTVD
KSRWQQGNVFSCSVMHEALHSHYTQKSLSLSPGK

SEQ ID NO: 41: mature human Fc IgG1, Cys to Ser substitution (#), DHS triple mutation (bold and underlined), allotype G1m(f) (bold italics), N-terminal Fab residues are underlined, hinge residues are italicized

NVNHKPSNTKVDKKVEPKSS(#)DKTHTCPPCPAPELLGGPSV
FLFPPKPKDTLMISRTPEVTCVVVDVSHEDPEVKFNWYVDGVE
VHNAKTKPREEQYNSTYRVVSVLTVDHHDWLNGKEYKCKVSNK
ALPAPIEKTISKAKGQPREPQVYTLPPSR [symbol] E [symbol] TKNQVSL
TCLVKGFYPSDIAVEWESNGQPENNYKTTPPVLDSDGSFFLYS
KLTVDKSRWQQGNVFSCSVMHEALHSHYTQKSLSLSPGK

SEQ ID NO: 42: mature human Fc IgG1, Cys to Ser substitution (#), DHS triple mutation (bold and underlined), allotype G1m(fa) (bold italics), N-terminal Fab residues are underlined, hinge residues are italicized

VNHKPSNTKVDKKVEPKSS(#)DKTHTCPPCPAPELLGGPSV
FLFPPKPKDTLMISRTPEVTCVVVDVSHEDPEVKFNWYVDGV
EVHNAKTKPREEQYNSTYRVVSVLTVDHHDWLNGKEYKCKVS
NKALPAPIEKTISKAKGQPREPQVYTLPPSR [symbol] E [symbol] TKNQ
VSLTCLVKGFYPSDIAVEWESNGQPENNYKTTPPVLDSDGSF
FLYSKLTVDKSRWQQGNVFSCSVMHEALHSHYTQKSLSLSPG

SEQ ID NO: 43: mature human Fc IgG1, Cys to Ser substitution (#), DHS triple mutation (bold and underlined), allotype G1m(f) (bold italics), N-terminal Fab residues are underlined, hinge residues are italicized

VNHKPSNTKVDKKVEPKSS(#)DKTHTCPPCPAPELLGGPSV
FLFPPKPKDTLMISRTPEVTCVVVDVSHEDPEVKFNWYVDGV
EVHNAKTKPREEQYNSTYRVVSVLTVDHHDWLNGKEYKCKVS
NKALPAPIEKTISKAKGQPREPQVYTLPPSR [symbol] E [symbol] TKNQ
VSLTCLVKGFYPSDIAVEWESNGQPENNYKTTPPVLDSDGSF
FLYSKLTVDKSRWQQGNVFSCSVMHEALHSHYTQKSLSLSPG

SEQ ID NO: 44: mature human Fc IgG1, Cys to Ser substitution (#), Asn to Ala substitution (*), DHS triple mutation (bold and underlined), wherein X₆ is Asp or Glu and X₇ is Leu or Met, N-terminal Fab residues are underlined, hinge residues are italicized

NVNHKPSNTKVDKKVEPKSS(#)DKTHTCPPCPAPELLGGPS
VFLFPPKPKDTLMISRTPEVTCVVVDVSHEDPEVKFNWYVDG
VEVHNAKTKPREEQYA(*)STYRVVSVLTVDHHDWLNGKEYK
CKVSNKALPAPIEKTISKAKGQPREPQVYTLPPSRX₆EX₇TKN
QVSLTCLVKGFYPSDIAVEWESNGQPENNYKTTPPVLDSDGS
FFLYSKLTVDKSRWQQGNVFSCSVMHEALHSHYTQKSLSLSP
GK

SEQ ID NO: 45: mature human Fc IgG1, Cys to Ser substitution (#), Asn to Ala substitution (*), DHS triple mutation (bold and underlined), allotype G1m(fa) (bold italics), N-terminal Fab residues are underlined, hinge residues are italicized

NVNHKPSNTKVDKKVEPKSS(#)DKTHTCPPCPAPELLGGPS
VFLFPPKPKDTLMISRTPEVTCVVVDVSHEDPEVKFNWYVDG
VEVHNAKTKPREEQYA(*)STYRVVSVLTVDHHDWLNGKEYK
CKVSNKALPAPIEKTISKAKGQPREPQVYTLPPSR [symbol] E [symbol]
TKNQVSLTCLVKGFYPSDIAVEWESNGQPENNYKTTPPVLD
SDGSFFLYSKLTVDKSRWQQGNVFSCSVMHEALHSHYTQKS
LSLSPGK

SEQ ID NO: 46: mature human Fc IgG1, Cys to Ser substitution (#), Asn to Ala substitution (*), DHS triple mutation (bold and underlined), allotype G1m(f) (bold italics), N-terminal Fab residues are underlined, hinge residues are italicized

NVNHKPSNTKVDKKVEPKSS(#)DKTHTCPPCPAPELLGG
PSVFLFPPKPKDTLMISRTPEVTCVVVDVSEDPEVKFNWY
VDGVEVHNAKTKPREEQYA(*)STYRVVSVLTVDHHDWLN
GKEYKCKVSNKALPAPIEKTIKAKGQPREPQVYTLPPSR [symbol]
E [symbol] TKNQVSLTCLVKGFYPSDIAVEWESNGQPENNY
KTTPPVLDSDGSFFLYSKLTVDKSRWQQGNVFSCSVMHEA
LHSHYTQKSLSLSPGK

SEQ ID NO: 47: mature human Fc IgG1, Cys to Ser substitution (#), Asn to Ala substitution (*), DHS triple mutation (bold and underlined), allotype G1m(fa) (bold italics), N-terminal Fab residues are underlined, hinge residues are italicized

VNHKPSNTKVDKKVEPKSS(#)DKTHTCPPCPAPELLGGPSVFLFPPKPK
DTLMISRTPEVTCVVVDVSHEDPEVKFNWYVDGVEVHNAKTKPREEQYA
(*)STYRVVSVLTVDHHDWLNGKEYKCKVSNKALPAPIEKTISKAKGQPR
EPQVYTLPPSR [symbol] E [symbol] TKNQVSLTCLVKGFYPSDIAVEWESNGQPENNYKTT
PPVLDSDGSFFLYSKLTVDKSRWQQGNVFSCSVMHEALHSHYTQKSLSLS
PGK

SEQ ID NO: 48: mature human Fc IgG1, Cys to Ser substitution (#), Asn to Ala substitution (*), DHS triple mutation (bold and underlined), allotype G1m(f) (bold italics), N-terminal Fab residues are underlined, hinge residues are italicized

VNHKPSNTKVDKKVEPKSS(#)DKTHTCPPCPAPELLGGPSVFLFPPKPK
DTLMISRTPEVTCVVVDVSHEDPEVKFNWYVDGVEVHNAKTKPREEQYA
(*)STYRVVSVLTVDHHDWLNGKEYKCKVSNKALPAPIEKTISKAKGQPR
EPQVYTLPPSR [symbol] E [symbol] TKNQVSLTCLVKGFYPSDIAVEWESNGQPENNYKTT
PPVLDSDGSFFLYSKLTVDKSRWQQGNVFSCSVMHEALHSHYTQKSLSLS
PGK

SEQ ID NO: 49: mature human Fc IgG1, Cys to Ser substitution (#), Asn to Ala substitution (*), DHS triple mutation (bold and underlined), allotype G1m(fa) (bold italics), N-terminal Fab residues are underlined, hinge residues are italicized

NVNHKPSNTKVDKKVEPKSS(#)DKTHTCPPCPAPELLGGPSVFLFPPKP
KDTLMISRTPEVTCVVVDVSHEDPEVKFNWYVDGVEVHNAKTKPREEQYA
(*)STYRVVSVLTVDHHDWLNGKEYKCKVSNKALPAPIEKTISKAKGQPR
EPQVYTLPPSR [symbol] E [symbol] TKNQVSLTCLVKGFYPSDIAVEWESNGQPENNYKTT
PPVLDSDGSFFLYSKLTVDKSRWQQGNVFSCSVMHEALHSHYTQKSLSLS
PGK

SEQ ID NO: 50: mature human Fc IgG1, Cys to Ser substitution (#), Asn to Ala substitution (*), DHS triple mutation (bold and underlined), allotype G1m(f) (bold italics), N-terminal Fab residues are underlined, hinge residues are italicized

NVNHKPSNTKVDKKVEPKSS(#)DKTHTCPPCPAPELLGGPSVFLFPPKP
KDTLMISRTPEVTCVVVDVSHEDPEVKFNWYVDGVEVHNAKTKPREEQYA
(*)STYRVVSVLTVDHHDWLNGKEYKCKVSNKALPAPIEKTISKAKGQPR
EPQVYTLPPSR [symbol] E [symbol] TKNQVSLTCLVKGFYPSDIAVEWESNGQPENNYKTT
PPVLDSDGSFFLYSKLTVDKSRWQQGNVFSCSVMHEALHSHYTQKSLSLS
PGK

SEQ ID NO: 51: mature human Fc IgG1, Cys to Ser substitution (#), Asn to Ala substitution (*), DHS triple mutation (bold and underlined), allotype G1m(fa) (bold italics), N-terminal Fab residues are underlined, hinge residues are italicized

VNHKPSNTKVDKKVEPKSS(#)DKTHTCPPCPAPELLGGPSVFLFPPKPK
DTLMISRTPEVTCVVVDVSHEDPEVKFNWYVDGVEVHNAKTKPREEQYA
(*)STYRVVSVLTVDHHDWLNGKEYKCKVSNKALPAPIEKTISKAKGQP
REPQVYTLPPSR [symbol] E [symbol] TKNQVSLTCLVKGFYPSDIAVEWESNGQPENNYK
TTPPVLDSDGSFFLYSKLTVDKSRWQQGNVFSCSVMHEALHSHYTQKSL
SLSPG

SEQ ID NO: 52: mature human Fc IgG1, Cys to Ser substitution (#), Asn to Ala substitution (*), DHS triple mutation (bold and underlined), allotype G1m(f) (bold italics), N-terminal Fab residues are underlined, hinge residues are italicized

VNHKPSNTKVDKKVEPKSS(#)DKTHTCPPCPAPELLGGPSVFLFPPKPK
DTLMISRTPEVTCVVVDVSHEDPEVKFNWYVDGVEVHNAKTKPREEQYA
(*)STYRVVSVLTVDHHDWLNGKEYKCKVSNKALPAPIEKTISKAKGQPR
EPQVYTLPPSR [symbol] E [symbol] TKNQVSLTCLVKGFYPSDIAVEWESNGQPENN
YKTTPPVLDSDGSFFLYSKLTVDKSRWQQGNVFSCSVMHEALHSHYTQKS
LSLSPG

SEQ ID NO: 53: mature human Fc IgG1, N-terminal ISAMVRS amino acid residues added (italicized), C-terminal G4S linker (italicized), C-terminal myc-tag (underlined), allotype G1m(f) (bold italics)

ISAMVRSDKTHTCPPCPAPELLGGPSVFLFPPKPKDTLMISRTPEVTCVV
VDVSHEDPEVKFNWYVDGVEVHNAKTKPREEQYNSTYRVVSVLTVLHQDW
LNGKEYKCKVSNKALPAPIEKTISKAKGQPREPQVYTLPPSR [symbol] E [symbol] TKN
QVSLTCLVKGFYPSDIAVEWESNGQPENNYKTTPPVLDSDGSFFLYSKLT
VDKSRWQQGNVFSCSVMHEALHNHYTQKSLSLSPGGGGGSEQKLISEEDL

SEQ ID NO: 54: mature human Fc IgG1, N-terminal ISAMVRS amino acid residues added (italicized), allotype G1m(fa) (bold italics)

KTHTCPPCPAPELLGGPSVFLFPPKPKDTLMISRTPEVTCVVVDVSHEDP
EVKFNWYVDGVEVHNAKTKPREEQYNSTYRVVSVLTVLHQDWLNGKEYKC
KVSNKALPAPIEKTISKAKGQPREPQVYTLPPSR [symbol] E [symbol] TKNQVSLTCLVKG
FYPSDIAVEWESNGQPENNYKTTPPVLDSDGSFFLYSKLTVDKSRWQQGN
VFSCSVMHEALHNHYTQKSLSLSPG

SEQ ID NO: 55: mature human Fc IgG1, N-terminal amino acid residues added (italicized), hinge residues are italicized allotype G1m(fa) (bold italics)

EPKSS(#)DKTHTCPPCPAPELLGGPSVFLFPPKPKDTLMISRTPEVTCV
VVDVSHEDPEVKFNWYVDGVEVHNAKTKPREEQYNSTYRVVSVLTVLHQD
WLNGKEYKCKVSNKALPAPIEKTISKAKGQPREPQVYTLPPSR [symbol] E [symbol] TKNQ
VSLTCLVKGFYPSDIAVEWESNGQPENNYKTTPPVLDSDGSFFLYSKLTV
DKSRWQQGNVFSCSVMHEALHNHYTQKSLSLSPG

SEQ ID NO: 56: mature human IgG1 Fc, Cys to Ser substitution (#), allotype G1m(fa) (bold italics), N-terminal Fab residues are underlined, hinge residues are italicized

NVNHKPSNTKVDKKVEPKSS(#)DKTHTCPPCPAPELLGGPSVFLFPPKP
KDTLMISRTPEVTCVVVDVSHEDPEVKFNWYVDGVEVHNAKTKPREEQYN
STYRVVSVLTVLHQDWLNGKEYKCKVSNKALPAPIEKTISKAKGQPREPQ
VYTLPPSR [symbol] E [symbol] TKNQVSLTCLVKGFYPSDIAVEWESNGQPENNYKTTPPV
LDSDGSFFLYSKLTVDKSRWQQGNVFSCSVMHEALHNHYTQKSLSLSPG

SEQ ID NO: 57: mature human IgG1 Fc, Cys to Ser substitution (#), allotype G1m(f) (bold italics), N-terminal Fab residues are underlined, hinge residues are italicized

NVNHKPSNTKVDKKVEPKSS(#)DKTHTCPPCPAPELLGGPSVFLFPPKP
KDTLMISRTPEVTCVVVDVSHEDPEVKFNWYVDGVEVHNAKTKPREEQYN
STYRVVSVLTVLHQDWLNGKEYKCKVSNKALPAPIEKTISKAKGQPREPQ
VYTLPPSR [symbol] E [symbol] TKNQVSLTCLVKGFYPSDIAVEWESNGQPENNYKTTPPV
LDSDGSFFLYSKLTVDKSRWQQGNVFSCSVMHEALHNHYTQKSLSLSPG

SEQ ID NO: 58: mature human IgG1 Fc, Cys to Ser substitution (#), M428L, N434S (Bold/Underlined), allotype G1m(fa) (bold italics), N-terminal Fab residues are underlined, hinge residues are italicized

NVNHKPSNTKVDKKVEPKSS(#)DKTHTCPPCPAPELLGGPSVFLFPPKP
KDTLMISRTPEVTCVVVDVSHEDPEVKFNWYVDGVEVHNAKTKPREEQYN
STYRVVSVLTVLHQDWLNGKEYKCKVSNKALPAPIEKTISKAKGQPREPQ
VYTLPPSR [symbol] E [symbol] TKNQVSLTCLVKGFYPSDIAVEWESNGQPENNYKTTP
PVLDSDGSFFLYSKLTVDKSRWQQGNVFSCSVLHEALHSHYTQKSLSLSPG

As defined herein, a variant Fc domain includes two variant Fc domain monomers that are dimerized by the interaction between the C_{H}3 antibody constant domains, as well as one or more disulfide bonds that form between the hinge domains of the two dimerizing variant Fc domain monomers. In some instances, a variant Fc domain forms the minimum structure that binds to an Fc receptor, e.g., Fc-gamma receptors (i.e., Fcγ receptors (FcγR)), Fc-alpha receptors (i.e., Fcα receptors (FcαR)), Fc-epsilon receptors (i.e., Fcε receptors (FcεR)), and/or the neonatal Fc receptor (FcRn). In some embodiments, an Fc domain of the present invention binds to an Fcγ receptor (e.g., FcRn, FcγRI (CD64), FcγRIIa (CD32), FcγRIIb (CD32), FcγRIIIa (CD16a), FcγRIIIb (CD16b)), and/or FcγRIV and/or the neonatal Fc receptor (FcRn).

In some embodiments, the variant Fc domain or variant Fc domain monomer of the invention is engineered to enhance binding to the neonatal Fc receptor (FcRn). Enhanced binding to the FcRn may increase the half-life Fc domain-containing conjugate or fusion protein, for example, the variant Fc domain monomer or variant Fc domain may increase the half-life of the conjugate by 5%, 10%, 15%, 20%, 30%, 40%, 50%, 60%, 70%, 80%, 90%. 100%, 200%, 300%, 400%, 500% or more relative to a conjugate having the corresponding Fc domain without the C220S/M252Y/S254T/T256E, C220S/V309D/Q311H/N434S, C220S, or further mutations that enhances FcRn binding. As used herein, an amino acid “corresponding to” a particular amino acid residue (e.g., of a particular SEQ ID NO.) should be understood to include any amino acid residue that one of skill in the art would understand to align to the particular residue (e.g., of the particular sequence). For example, any one of SEQ ID NOs: 1-3, 8-13, or 20-29 may be mutated to include an N297 (e.g., N297A) mutation by mutating the “corresponding residues” of the amino acid sequence.

In some instances, the variant Fc domain or variant Fc domain monomer of the invention is engineered to reduce or ablate binding to an Fc receptor, e.g., Fc-gamma receptors (i.e., Fcγ receptors (FcγR)), Fc-alpha receptors (i.e., Fcα receptors (FcαR)), Fc-epsilon receptors (i.e., Fcε receptors (FcεR)), and/or the neonatal Fc receptor (FcRn). In some embodiments, an Fc domain of the present invention binds to an Fcγ receptor (e.g., FcRn, FcγRI (CD64), FcγRIIa (CD32), FcγRIIb (CD32), FcγRIIIa (CD16a), FcγRIIIb (CD16b)), and/or FcγRIV and/or the neonatal Fc receptor (FcRn) and are particularly useful for the delivery of therapeutic agents (e.g., small molecule therapeutic agents and therapeutic peptide agents).

In some embodiments, the variant Fc domain or variant Fc domain monomer of the invention has the sequence of any one of SEQ ID NOs: 1-29 and 31-52 may further include additional amino acids at the N-terminus (Xaa)x and/or additional amino acids at the C-terminus (Xaa)z, wherein each Xaa is independently any amino acid and x and z are a whole number greater than or equal to zero, generally less than 100, preferably less than 10 and more preferably 0, 1, 2, 3, 4, or 5.

### Activation of Immune Cells

Fc-gamma receptors (FcγRs) bind the Fc portion of immunoglobulin G (IgG) and play important roles in immune activation and regulation. For example, the IgG Fc domains in immune complexes (ICs) engage FcγRs with high avidity, thus triggering signaling cascades that regulate immune cell activation. The human FcγR family contains several activating receptors (FcγRI, FcγRIIa, FcγRIIc, FcγRIIIa, and FcγRIIIb) and one inhibitory receptor (FcγRIIb). FcγR signaling is mediated by intracellular domains that contain immune tyrosine activating motifs (ITAMs) for activating FcγRs and immune tyrosine inhibitory motifs (ITIM) for inhibitory receptor FcγRIIb. In some embodiments, FcγR binding by Fc domains results in ITAM phosphorylation by Src family kinases; this activates Syk family kinases and induces downstream signaling networks, which include PI3K and Ras pathways.

In some instances, in the conjugates and fusion proteins described herein, the portion of the conjugates or fusion proteins including monomers or dimers of a therapeutic agent bind to a surface exposed target of an infectious pathogen (e.g., a viral particle, a fungi, or a bacterium), while the variant Fc domain portion of the conjugates or fusion proteins bind to FcγRs (e.g., FcRn, FcγRI, FcγRIIa, FcγRIIc, FcγRIIIa, and FcγRIIIb) on immune cells and activate phagocytosis and effector functions, such as antibody-dependent cell-mediated cytotoxicity (ADCC), thus leading to the engulfment and destruction of infectious pathogen by immune cells and further enhancing the antipathogenic (e.g., antiviral, antifungal, or antibacterial) activity of the conjugates. Examples of immune cells that may be activated by the conjugates described herein include, but are not limited to, macrophages, neutrophils, eosinophils, basophils, lymphocytes, follicular dendritic cells, natural killer cells, and mast cells.

### Half-Life

Biological half-life (t_{1/2}) is the time it takes a therapeutic to decrease its maximum concentration by half. Improvements in half-life for therapeutics can lower the efficacious dose. There are many variables that affect half-life from patient variables (e.g., age. blood circulation, diet, excessive fluids, low fluids, gender, history of drug use, kidney function, liver function, obesity, pre-existing conditions etc.) to therapeutic specific variables (e.g., therapeutic formulation, pharmacokinetics, administration method, drug clearance (e.g., kidney, liver, or lungs), tissue distribution and accumulation, therapeutic size, charge, pKa, etc.). For peptide therapeutics short plasma half-lives are commonly due to fast renal clearance as well as to enzymatic degradation occurring during systemic circulation. Modifications of the peptide or protein can lead to prolonged plasma half-life times. In some instances, the variant Fc domain or fusion protein are engineered to increase the half-life of the variant Fc domain monomer, conjugate, or fusion protein. In some embodiments, the variant Fc domain or variant Fc domain monomer of the invention is engineered to enhance binding to the neonatal Fc receptor (FcRn). Enhanced binding to the FcRn may increase the half-life Fc domain-containing conjugate or fusion protein, for example, the variant Fc domain monomer or variant Fc domain may increase the half-life of the conjugate by 5%, 10%, 15%, 20%, 30%, 40%, 50%, 60%, 70%, 80%, 90%. 100%, 200%, 300%, 400%, 500% or more relative to a conjugate having the corresponding Fc domain without a mutation, e.g., the C220S/M252Y/S254T/T256E, C220S/V309D/Q311H/N434S, or further mutations that enhances FcRn binding. In some instances, the variant Fc domain monomer is engineered to include at least 220 residues.

### Renal Clearance

Many therapeutic peptides have short half-lives (minutes) in vivo due to their size. The rapid clearance and short half-life of peptides limit their development into successful drugs. One of the main causes of rapid clearance of peptides from systemic circulation is renal clearance. The glomeruli have a pore size of approximately 8 nm, and hydrophilic peptides with MW<2-25 kDa are susceptible to rapid filtration through the glomeruli of the kidney. In some embodiments, the variant Fc domain monomers and fusion proteins described herein are greater than 20 kDa. In some embodiments, the variant Fc domain monomers and fusion proteins of two conjugates or fusion proteins may dimerize to form a variant Fc domain. In some embodiments, the variant Fc domain monomer, the conjugate, or the fusion protein are engineered to decrease renal clearance. Decreased renal clearance may increase the half-life of the variant Fc domain monomer of a conjugate or fusion protein described herein, for example, the variant Fc domain may include at least about 200 amino acids (e.g., at least 200, at least 225, at least about 230, at least about 240, at least about 242, at least about 243, at least about 250, at least about 255, at least about 260, at least about 265, at least about 270, at least about 275, at least about 280, at least about 285, at least about 290, at least about 295, or at least about 300 amino acids).

### Tissue Distribution

After a therapeutic enters the systemic circulation, it is distributed to the body's tissues. Distribution is generally uneven because of different in blood perfusion, tissue binding, regional Ph, and permeability of cell membranes. The entry rate of a drug into a tissue depends on the rate of blood flow to the tissue, tissue mass, and partition characteristics between blood and tissue. Distribution equilibrium (when the entry and exit rates are the same) between blood and tissue is reached more rapidly in richly vascularized areas, unless diffusion across cell membranes is the rate-limiting step. The size, shape, charge, target binding, FcRn and target binding mechanisms, route of administration, and formulation affect tissue distribution.

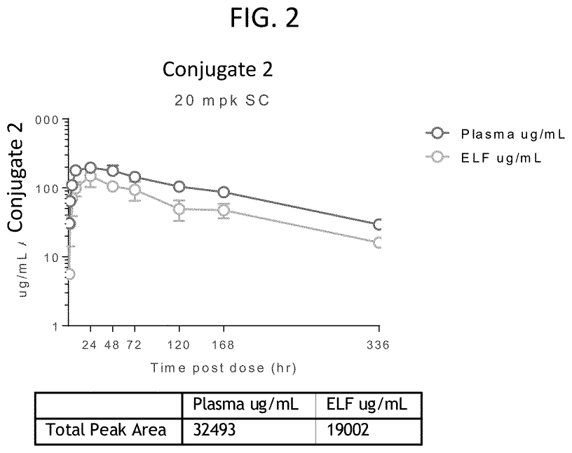

In some instances, the variant Fc polypeptide is optimized to distribute to lung tissue. In some instances, the variant Fc domain monomers, conjugates, and fusion proteins have a concentration ratio of distribution in epithelial lining fluid of at least 30% the concentration of the polypeptide, the conjugate, or the fusion protein in plasma within 2 hours after administration. In certain embodiments, ratio of the concentration is at least 45% within 2 hours after administration. In some embodiments, the ratio of concentration is at least 55% within 2 hours after administration. In particular, the ratio of concentration is at least 60% within 2 hours after administration. As shown in Example 5 and FIG. 2, by 2 hours post injection, conjugate 2 ELF levels are surprisingly ˜60% of plasma exposure levels as measured by AUC across the rest of the time course indicating nearly immediate partitioning of conjugate 2 from plasma to the ELF in the lung. This demonstrates that conjugate 2 rapidly distributes to lung, and maintains high concentrations in lung relative to levels in plasma.

In some embodiments, the variant Fc domain monomer includes 400 amino acid residues or less, 350 amino acid residues or less, 300 amino acid residues or less, or 250 amino acid residues or less.

In some instances, the variant Fc polypeptide is optimized to distribute to hepatic, neural (e.g., CNS), muscular, dermal, ocular, or vascular tissue.

Where the Fc polypeptide preferentially distributes to one or more particular tissues, the polypeptide may be used to treat disorders of the corresponding tissue (e.g., deliver a therapeutic agent to the tissue).

### Boundaries of Fc Domain Monomer

The length (e.g., as determined by the N-terminal and C-terminal boundaries) of the variant Fc domain monomer may be optimized in order to prevent renal clearance and increase distribution to a desired tissue (e.g., lung tissue). Antibodies are divided into two domains: the Fc (effector) domain and the fragment antigen-binding (Fab) domain, the latter of which contains the antigen-binding regions. The present disclosure provides variant Fc domain monomers which include a portion of the Fab domain at the N-terminus of the Fc domain. The inventors have observed that smaller Fc constructs (e.g., Fc constructs lacking a portion of the Fab domain) demonstrated a decreased half-life, likely due to renal elimination. To address this problem, the Fc constructs were iteratively lengthened by adding back in some of the Fab domain on the N-terminus, until further increases in size did not lead to improvements (e.g., in mouse pharmacokinetic experiments). The present disclosure provides variant Fc domain monomers which have been optimized (e.g., by length, mass, N-terminal, and/or C-terminal boundaries in addition to mutational variants) to achieve the desired increased half-life and/or tissue distribution.

In some embodiments, the N-terminus of the variant Fc domain monomer includes between 10 and 20 residues (e.g., 11, 12, 13, 14, 15, 16, 17, 18, or 19 residues) of the Fab domain. In certain embodiments, the N-terminus of the variant Fc domain monomer is any one of amino acid residues 198-205. In some embodiments, the N-terminus of the variant Fc domain monomer is amino acid residue 201 (e.g., Asn 201). In certain embodiments, the N-terminus of the variant Fc domain monomer is amino acid residue 202 (e.g., Val 202). In other embodiments, the C-terminus of the variant Fc domain monomer is any one of amino acid residues 437-447. In another embodiment, the C-terminus of the variant Fc domain monomer is amino acid residue 446 (e.g., Gly 446). In some embodiments, the C-terminus of the variant Fc domain monomer is amino acid residue 447 (e.g. Lys 447).

Lengthening the construct required the addition of a portion of the hinge region that contains a free cysteine residue (C220), which created issues with thiol mediated aggregation. C220 was mutated to a serine (C220S) to avert this problem.

### Therapeutic Agent Delivery

The large size of antibody molecules can make it difficult to transport targeting systems across cellular membranes. In some instances, large targeting systems can lead to slow elimination from the blood circulation, which can ultimately lead to myelotoxicity. In addition, in vivo use of antibody-based targeting systems is expensive and can lead to immunogenicity after repeated injections of such formulations. Antibody fragments which are smaller than whole antibodies have successfully been made but are still, in many instances, too large. Fragments can reach extracellular spaces more easily than whole antibodies. In some instances, the variant Fc domain monomers can be used in conjugates to deliver a therapeutic agent. In some instances, variant Fc domain forms the minimum structure that binds to an Fc receptor, e.g., Fc-gamma receptors (i.e., Fcγ receptors (FcγR)), Fc-alpha receptors (i.e., Fcα receptors (FcαR)), Fc-epsilon receptors (i.e., Fcε receptors (FcεR)), and/or the neonatal Fc receptor (FcRn). In some embodiments, an Fc domain of the present invention binds to an Fcγ receptor (e.g., FcRn, FcγRI (CD64), FcγRIIa (CD32), FcγRIIb (CD32), FcγRIIIa (CD16a), FcγRIIIb (CD16b)), and/or FcγRIV and/or the neonatal Fc receptor (FcRn). Binding of the neonatal Fc receptor mediates internalization of the variant Fc domain monomer or conjugate of fusion protein thereof, thereby delivering a therapeutic agent to a cell. Upon internalization, an endocytic salvage pathway that prevents degradation of the variant Fc domain monomer or conjugate or fusion protein thereof. In some instances, the variant Fc domain monomer of variant Fc domain is engineered to reduce neonatal Fc receptor binding, thereby decreasing internalization into a cell and increasing the plasma concentration of the variant Fc domain conjugate or fusion protein thereof.

### II. Conjugates of the Disclosure

Provided herein are synthetic conjugates useful in the treatment of a condition or disorder described herein (e.g., a respiratory disorder, a hepatic disorder, a central nervous system disorder, a muscular disorder, a skin disorder, an ocular disorder, a vascular disorder, or an infection (e.g., a viral infection, a fungal infection, or a bacterial infection)). The conjugates disclosed herein (e.g., conjugates described by formula (1)), include a variant Fc domain conjugated to one or more therapeutic agents (e.g., one or more small molecule therapeutic agents).

Without being bound by theory, in some aspects, conjugates described herein bind to a surface exposed target of an infectious pathogen (e.g., a viral particle, a fungi, or a bacterium) through the interactions between the therapeutic agent in the conjugates and proteins on the surface of the infectious pathogen.

Conjugates of the invention include therapeutic agents conjugated to a variant Fc domain or variant Fc domain monomer. The variant Fc domain in the conjugates described herein binds to the FcγRs (e.g., FcRn, FcγRI, FcγRIIa, FcγRIIc, FcγRIIIa, and FcγRIIIb) on immune cells. The binding of the variant Fc domain in the conjugates described herein to the FcγRs on immune cells activates phagocytosis and effector functions, such as antibody-dependent cell-mediated cytotoxicity (ADCC), thus leading to the engulfment and destruction of infectious pathogen by immune cells and further enhancing the activity of the conjugates.

In some embodiments, a conjugate provided herein is described by formula (1). In some embodiments, when n is 2, E (a variant Fc domain monomer) dimerizes to form a variant Fc domain.

In some embodiments, the variant Fc domain monomer of the conjugate includes less than about 300 amino acid residues (e.g., less than about 300, less than about 295, less than about 290, less than about 285, less than about 280, less than about 275, less than about 270, less than about 265, less than about 260, less than about 255, less than about 250, less than about 245, less than about 240, less than about 235, less than about 230, less than about 225, or less than about 220 amino acid residues). In some embodiments, the variant Fc domain monomer of the conjugate is less than about 40 kDa (e.g., less than about 35 kDa, less than about 30 kDa, less than about 25 kDa).

In some embodiments, the variant Fc domain monomer of the conjugate includes at least 200 amino acid residues (e.g., at least 210, at least 220, at least 230, at least 240, at least 250, at least 260, at least 270, at least 280, at least 290, or at least 300 amino residues). In some embodiments, the variant Fc domain monomer is at least 20 kDa (e.g., at least 25 kDa, at least 30 kDa, or at least 35 kDa).

In some embodiments, the variant Fc domain monomer of the conjugate includes 200 to 400 amino acid residues (e.g., 200 to 250, 250 to 300, 300 to 350, 350 to 400, 200 to 300, 250 to 350, or 300 to 400 amino acid residues). In some embodiments, the variant Fc domain monomer of the conjugate is between 200 and 300 amino acid residues (e.g., between 210 and 300, between 230 and 300, between 250 and 300, between 270 and 300, between 290 and 300, between 210 and 290, between 220 and 280, between 230 and 270, between 240 and 260, or between 245 and 255 amino acid residues) in length. In some embodiments, the variant Fc domain monomer of the conjugate is 20 to 40 kDa (e.g., 20 to 25 kDa, 25 to 30 kDa, 35 to 40 kDa, 20 to 30 kDa, 25 to 35 kDa, or 30 to 40 KDa). In some embodiments, the variant Fc domain monomer of the conjugate is between about 20 kDa and about 40 kDa (e.g., 20 kDa to 25 kDa, 25 kDa to 30 kDa, 30 kDa to 35 kDa, 35 kDa to 40 kDa) in mass

In some embodiments, each linker includes a polyethylene glycol (PEG) linker including between about 2-10 (e.g., 2, 3, 4, 5, 6, 7, 8, 9, or 10) PEG units. In some embodiments, at least one arm of the trivalent linker includes a polyethylene glycol (PEG) linker including between about 2-10 (e.g., 2, 3, 4, 5, 6, 7, 8, 9, or 10) PEG units.

In some embodiments, the conjugate is at least 40 kDa (e.g., at least 45 kDa, at least 50 kDa, at least 55 kDa, at least 60 kDa, at least 65 kDa, at least 70 kDa, at least 75 kDa, or at least 80 kDa). In some embodiments, the conjugate is between about 40 kDa and about 80 kDa (e.g., 40 kDa to 50 kDa, 45 kDa to 55 kDa, 50 kDa to 60 kDa, 55 kDa to 65 kDa, 60 kDa to 70 kDa, 65 kDa to 75 kDa, or 70 kDa to 80 kDa) in mass.

In particular embodiments, the conjugate includes a variant Fc domain monomer including between 230 to 250 amino acid residues (e.g., 231 amino acid residues, 232 amino acid residues, 233 amino acid residues, 234 amino acid residues, 235 amino acid residues, 236 amino acid residues, 237 amino acid residues, 238 amino acid residues, 239 amino acid residues, 240 amino acid residues, 241 amino acid residues, 242 amino acid residues, 243 amino acid residues, 244 amino acid residues, 245 amino acid residues, 246 amino acid residues, 247 amino acid residues, 248 amino acid residues, 249 amino acid residues, or 250 amino acid residues), linked to between an average of 1 to 10 (e.g., 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, or 10) small molecules by way of a linker (e.g., a dimeric linker or a trimeric linker (e.g., a linker including between 2-10 PEG units) linked to one or more (e.g., 1, 2, 3, 4, or more)) small molecules.

Conjugates described herein may be synthesized using available chemical synthesis techniques in the art. In cases where a functional group is not available for conjugation, a molecule may be derivatized using conventional chemical synthesis techniques that are well known in the art. In some embodiments, the conjugates described herein contain one or more chiral centers. The conjugates include each of the isolated stereoisomeric forms as well as mixtures of stereoisomers in varying degrees of chiral purity, including racemic mixtures. It also encompasses the various diastereomers, enantiomers, and tautomers that can be formed.

In the conjugates described herein, the squiggly line connected to E indicates that one or more (e.g., 1, 2, 3, 4, 5, 6, 7, 8, 9, 10, 11, 12, 13, 14, 15, 16, 17, 18, 19, or 20) therapeutic agents may be attached to a variant Fc domain monomer. In some embodiments, when n is 1, one or more (e.g., 1, 2, 3, 4, 5, 6, 7, 8, 9, or 10) therapeutic agents may be attached to variant Fc domain monomer or variant Fc domain. In some embodiments, when n is 2, one or more (e.g., 1, 2, 3, 4, 5, 6, 7, 8, 9, 10, 11, 12, 13, 14, 15, 16, 17, 18, 19, or 20) therapeutic agents may be attached to a variant Fc domain. The squiggly line in the conjugates described herein is not to be construed as a single bond between one or more therapeutic agents and an atom in the variant Fc domain. In some embodiments, when T is 1, one therapeutic agent may be attached to an atom in the variant Fc domain monomer or variant Fc domain. In some embodiments, when T is 2, two therapeutic agents may be attached to an atom in the variant Fc domain monomer or variant Fc domain.

As described further herein, a linker in a conjugate described herein (e.g., L) may be a branched structure. As described further herein, a linker in a conjugate described herein (e.g., L) may be a multivalent structure, e.g., a divalent or trivalent structure having two or three arms, respectively. In some embodiments when the linker has three arms, two of the arms may be attached to the first and second therapeutic agent and the third arm may be attached to the variant Fc domain monomer or variant Fc domain.

In conjugates having a variant Fc domain covalently linked to one or more therapeutic agents, as represented by the formula (1), when n is 2, two variant Fc domain monomers (each variant Fc domain monomer is represented by E) dimerize to form a variant Fc domain.

### Conjugates of Monomers of a Therapeutic Agent Linked to Variant Fc Domain

In some embodiments, the conjugates described herein include a variant Fc domain monomer or variant Fc domain covalently linked to one or more monomers of a therapeutic agent. Conjugates of variant Fc domain monomer and one or more monomers of a therapeutic agent may be formed by linking the variant Fc domain to each of the monomers of a therapeutic agent through a linker, such as any of the linkers described herein.

In the conjugates having a variant Fc domain covalently linked to one or more monomers of a therapeutic agent described herein, the squiggly line connected to E indicates that one or more (e.g., 1, 2, 3, 4, 5, 6, 7, 8, 9, 10, 11, 12, 13, 14, 15, 16, 17, 18, 19, or 20) monomers of a therapeutic agent may be attached to a variant Fc domain monomer or variant Fc domain. In some embodiments, when n is 1, one or more (e.g., 1, 2, 3, 4, 5, 6, 7, 8, 9, or 10) monomers of a therapeutic agent may be attached to a variant Fc domain monomer. In some embodiments, when n is 2, one or more (e.g., 1, 2, 3, 4, 5, 6, 7, 8, 9, 10, 11, 12, 13, 14, 15, 16, 17, 18, 19, or 20) monomers of a therapeutic agent may be attached to a variant Fc domain. The squiggly line in the conjugates described herein is not to be construed as a single bond between one or more monomers of a therapeutic agent and an atom in the variant Fc domain monomer or variant Fc domain. In some embodiments, when T is 1, one monomer of a therapeutic agent may be attached to an atom in the variant Fc domain monomer or variant Fc domain. In some embodiments, when T is 2, two monomers of a therapeutic agent may be attached to an atom in the variant Fc domain monomer or variant Fc domain. In some embodiments, the conjugated variant Fc domain is part of a fusion protein described herein.

In some embodiments, the first A-L moiety is conjugated specifically to lysine residues of E (e.g., the nitrogen atoms of surface exposed lysine residues of E), and the second A-L moiety is conjugated specifically to cysteine residues of E (e.g., the sulfur atoms of surface exposed cysteine residues of E). In some embodiments, the first A-L moiety is conjugated specifically to cysteine residues of E (e.g., the sulfur atoms of surface exposed cysteine residues of E), and the second A-L moiety is conjugated specifically to lysine residues of E (e.g., the nitrogen atoms of surface exposed lysine residues of E).

As described further herein, a linker in a conjugate having a variant Fc domain monomer or variant Fc domain covalently linked to one or more a therapeutic agents described herein (e.g., L) may be a divalent structure having two arms. One arm in a divalent linker may be attached to the therapeutic agents and the other arm may be attached to the variant Fc domain monomer or variant Fc domain.

In conjugates having a variant Fc domain covalently linked to one or more monomers of a therapeutic agent, as described herein, when n is 2, two variant Fc domain monomers (each variant Fc domain monomer is represented by E) dimerize to form a variant Fc domain.

### Conjugates of Dimers of a Therapeutic Agent Linked to Variant Fc Domain

In some embodiments, the conjugates described herein (e.g., conjugates of formula (1)) include a variant Fc domain monomer or variant Fc domain covalently linked to one or more dimers of a therapeutic agent. Conjugates of a variant Fc domain monomer and one or more dimers of a therapeutic agent may be formed by linking the variant Fc domain to each of the dimers of a therapeutic agent through a linker, such as a linker described herein. The first and second therapeutic agents are linked to each other by way of a linker, such as a linker described herein. In some embodiments, where the therapeutic agent is a dimer each therapeutic agent can be the same small molecule agent (e.g., a homodimer) or a different small molecule agent (e.g., a hetero dimer).

In the conjugates having a variant Fc domain covalently linked to one or more dimers of a therapeutic agent described herein, the squiggly line connected to E indicates that one or more (e.g., 1, 2, 3, 4, 5, 6, 7, 8, 9, 10, 11, 12, 13, 14, 15, 16, 17, 18, 19, or 20) dimers of therapeutic agents may be attached to a variant Fc domain monomer or variant Fc domain. In some embodiments, when n is 1, one or more (e.g., 1, 2, 3, 4, 5, 6, 7, 8, 9, or 10) dimers of therapeutic agents may be attached to a variant Fc domain monomer. In some embodiments, when n is 2, one or more (e.g., 1, 2, 3, 4, 5, 6, 7, 8, 9, 10, 11, 12, 13, 14, 15, 16, 17, 18, 19, or 20) dimers of therapeutic agents may be attached to a variant Fc domain. The squiggly line in the conjugates described herein is not to be construed as a single bond between one or more dimers of therapeutic agents and an atom in the variant Fc domain monomer or variant Fc domain. In some embodiments, when T is 1, one dimer of therapeutic agents may be attached to an atom in the variant Fc domain monomer or variant Fc domain. In some embodiments, when T is 2, two monomers of a therapeutic agent may be attached to an atom in the variant Fc domain monomer or variant Fc domain. In some embodiments, the variant Fc domain is part of a fusion protein described herein.

In some embodiments, the first A-L moiety is conjugated specifically to lysine residues of E (e.g., the nitrogen atoms of surface exposed lysine residues of E), and the second A-L moiety is conjugated specifically to cysteine residues of E (e.g., the sulfur atoms of surface exposed cysteine residues of E). In some embodiments, the first A-L moiety is conjugated specifically to cysteine residues of E (e.g., the sulfur atoms of surface exposed cysteine residues of E), and the second A-L moiety is conjugated specifically to lysine residues of E (e.g., the nitrogen atoms of surface exposed lysine residues of E).

As described further herein, a linker in a conjugate having a variant Fc domain monomer or variant Fc domain covalently linked to one or more dimers of therapeutic agents described herein (e.g., L) may be a trivalent structure (e.g., a trivalent linker). A trivalent linker has three arms, in which each arm is covalently linked to a component of the conjugate (e.g., a first arm conjugated to a first therapeutic agent, a second arm conjugated to a therapeutic agent, and a third arm conjugated to the fusion protein or the variant Fc domain monomer).

In conjugates having a variant Fc domain covalently linked to one or more dimers of therapeutic agents, as described herein, when n is 2, two variant Fc domain monomers (each variant Fc domain monomer is represented by E) dimerize to form a variant Fc domain.

### III. Fusion Proteins

The invention features fusion proteins which include at least one variant Fc domain monomer conjugated to at least one (e.g., one or two) therapeutic peptide agents. An exemplary fusion protein of the invention includes the structure: (P₂-L₂)n₂-B-(L₁-P₁)n₁, wherein B is a variant Fc domain monomer (e.g., and Fc domain monomer including the amino acid sequence of any one of SEQ ID NOs: 1-29, 31-52, and 56-58) or a conjugate thereof; P₁ and P₂ are each independently a therapeutic peptide agent; L₁ and L₂ are each independently a linker (e.g., a chemical linker or a peptide linker); and n₁ and n₂ are each independently 0 or 1, wherein at least one of n₁ and n₂ is 1 (e.g., the fusion protein must include at least one therapeutic peptide agent).

In some embodiments, the fusion protein includes one variant Fc domain monomer conjugated to one therapeutic peptide agent. For example, n₁ is 1, n₂ is 0, and the fusion protein includes the structure: B-L₁-P₁. The variant Fc domain monomer and the therapeutic peptide agent may be conjugated in any orientation. Where a C-to-N conjugation occurs, the variant Fc domain monomer and the therapeutic peptide agent may be expressed as a single polypeptide construct including a polypeptide linker or may be expressed separately and subsequently conjugated via a polypeptide or chemical linker. Where an C-to-C or N-to-N conjugation occurs, the variant Fc domain monomer and the therapeutic peptide agent are expressed separately and subsequently conjugated, e.g., via a chemical or peptide linker. For example, the linker (L₁) may be conjugated to C-terminus of the variant Fc domain monomer (B) and to the N-terminus of the therapeutic peptide agent (P₁). Alternately, the linker (L₁) may be conjugated to N-terminus of the variant Fc domain monomer (B) and to the C-terminus of the therapeutic peptide agent (P₁). Alternately, the linker (L₁) is conjugated to N-terminus of the variant Fc domain monomer (B) and to the N-terminus of the therapeutic peptide agent (P₁). Alternately, the linker (L₁) is conjugated to C-terminus of the variant Fc domain monomer (B) and to the C-terminus of the therapeutic peptide agent (P₁).

In some embodiments, the fusion protein includes one variant Fc domain monomer conjugated to two therapeutic peptide agents. For example, n₁ is 1, n₂ is 1, and the fusion protein includes the structure: P₂-L₂-B-L₁-P₁. As described above, conjugation can occur in any orientation, and the fusion protein may be expressed as a singly polypeptide construct, or may be assembled by chemical conjugation. For example, the linker (L₂) may be conjugated to the C-terminus of the therapeutic peptide agent (P₂) and to the N-terminus of the variant Fc domain monomer (B), and the linker (L₁) may be conjugated to the C-terminus of the variant Fc domain monomer (B) and to the N-terminus of the therapeutic peptide agent (P₁). Alternately, the linker (L₂) may be conjugated to the N-terminus of the therapeutic peptide agent (P₂) and to the N-terminus of the variant Fc domain monomer (B), and the linker (L₁) may be conjugated to the N-terminus of the therapeutic peptide agent (P₁) and to the C-terminus of the variant Fc domain monomer (B). Alternately, the linker (L₂) may be conjugated to the C-terminus of the therapeutic peptide agent (P₂) and to the N-terminus of the variant Fc domain monomer (B), and the linker (L₁) may be conjugated to the C-terminus of the therapeutic peptide agent (P₁) and to the C-terminus of the variant Fc domain monomer (B).

The disclosure also provides a conjugate including a first fusion protein selected from any of the therapeutic peptide agent-variant variant Fc domain monomer fusion proteins described herein; and a second fusion protein selected from any of the therapeutic peptide agent-variant variant Fc domain monomer fusion proteins described herein; wherein the variant Fc domain monomer (B) of the first fusion protein and the variant Fc domain monomer (B) of the second fusion protein dimerize to form an variant Fc domain monomer. In some embodiments, the first fusion protein and the second fusion protein have the same structure and the conjugate is a homodimer.

### IV. Linkers

A linker refers to a linkage or connection between two or more components in a conjugate described herein (e.g., between two therapeutic agents in a conjugate described herein, between a therapeutic agent and a variant Fc domain monomer or variant Fc domain in a conjugate described herein, and between a dimer of two therapeutic agents and a variant Fc domain monomer or variant Fc domain in a conjugate described herein).

A linker can be a simple covalent bond, e.g., a peptide bond, a synthetic polymer, e.g., a polyethylene glycol (PEG) polymer, or any kind of bond created from a chemical reaction, e.g. chemical conjugation. In the case that a linker is a peptide bond, the carboxylic acid group at the C-terminus of one protein domain can react with the amino group at the N-terminus of another protein domain in a condensation reaction to form a peptide bond. Specifically, the peptide bond can be formed from synthetic means through a conventional organic chemistry reaction well-known in the art, or by natural production from a host cell, wherein a polynucleotide sequence encoding the DNA sequences of both proteins, e.g., two variant Fc domain monomers, in tandem series can be directly transcribed and translated into a contiguous polypeptide encoding both proteins by the necessary molecular machineries, e.g., DNA polymerase and ribosome, in the host cell.

In the case that a linker is a synthetic polymer, e.g., a PEG polymer, the polymer can be functionalized with reactive chemical functional groups at each end to react with the terminal amino acids at the connecting ends of two proteins.

In the case that a linker (except peptide bond mentioned above) is made from a chemical reaction, chemical functional groups, e.g., amine, carboxylic acid, ester, azide, or other functional groups commonly used in the art, can be attached synthetically to the C-terminus of one protein and the N-terminus of another protein, respectively. The two functional groups can then react to through synthetic chemistry means to form a chemical bond, thus connecting the two proteins together. Such chemical conjugation procedures are routine for those skilled in the art.

### Peptide Linkers

In the present invention, a linker between a therapeutic peptide agent and a variant Fc domain monomer (e.g. L₁ or L₂) can be polypeptide including 3-200 amino acids (e.g., 3-200, 3-180, 3-160, 3-140, 3-120, 3-100, 3-90, 3-80, 3-70, 3-60, 3-50, 3-45, 3-40, 3-35, 3-30, 3-25, 3-20, 3-15, 3-10, 3-9, 3-8, 3-7, 3-6, 3-5, 3-4, 4-200, 5-200, 6-200, 7-200, 8-200, 9-200, 10-200, 15-200, 20-200, 25-200, 30-200, 35-200, 40-200, 45-200, 50-200, 60-200, 70-200, 80-200, 90-200, 100-200, 120-200, 140-200, 160-200, or 180-200 amino acids). In some embodiments, a linker between a therapeutic peptide agent and a variant Fc domain monomer (e.g. L₁ or L₂) is a polypeptide containing at least 12 amino acids, such as 12-200 amino acids (e.g., 12-200, 12-180, 12-160, 12-140, 12-120, 12-100, 12-90, 12-80, 12-70, 12-60, 12-50, 12-40, 12-30, 12-20, 12-19, 12-18, 12-17, 12-16, 12-15, 12-14, or 12-13 amino acids) (e.g., 14-200, 16-200, 18-200, 20-200, 30-200, 40-200, 50-200, 60-200, 70-200, 80-200, 90-200, 100-200, 120-200, 140-200, 160-200, 180-200, or 190-200 amino acids). In some embodiments, a linker between a therapeutic peptide agent and a variant Fc domain monomer (e.g. L₁ or L₂) is a polypeptide containing 12-30 amino acids (e.g., 12, 13, 14, 15, 16, 17, 18, 19, 20, 21, 22, 23, 24, 25, 26, 27, 28, 29, or 30 amino acids).

Suitable peptide linkers are known in the art, and include, for example, peptide linkers containing flexible amino acid residues such as glycine and serine. In some embodiments, a linker can contain motifs, e.g., multiple or repeating motifs, of GS, GGS (SEQ ID NO: 61), GGGGS (SEQ ID NO: 62), GGSG (SEQ ID NO: 63), or SGGG (SEQ ID NO: 64). In some embodiments, a linker can contain 2 to 12 amino acids including motifs of GS, e.g., GS, GSGS (SEQ ID NO: 65), GSGSGS (SEQ ID NO: 66), GSGSGSGS (SEQ ID NO: 67), GSGSGSGSGS (SEQ ID NO: 68), or GSGSGSGSGSGS (SEQ ID NO: 69). In some other embodiments, a linker can contain 3 to 12 amino acids including motifs of GGS, e.g., GGS, GGSGGS (SEQ ID NO: 70), GGSGGSGGS (SEQ ID NO: 71), and GGSGGSGGSGGS (SEQ ID NO: 72). In yet other embodiments, a linker can contain 4 to 12 amino acids including motifs of GGSG (SEQ ID NO: 73), e.g., GGSGGGSG (SEQ ID NO: 74), or GGSGGGSGGGSG (SEQ ID NO: 75). In other embodiments, a linker can contain motifs of GGGGS (SEQ ID NO: 61), e.g., GGGGSGGGGSGGGGS (SEQ ID NO: 76). In some embodiments, a linker is SGGGSGGGSGGGSGGGSGGG (SEQ ID NO: 77).

In preferred embodiments, a peptide linker (e.g., L₁ and L₂) is a peptide linker including the amino acid sequence of any one of (GS)x, (GGS)x, (GGGGS)x, (GGSG)x, (SGGG)x, wherein x is an integer from 1 to 50 (e.g., 1-40, 1-30, 1-20, 1-10, or 1-5).

In some embodiments, a peptide linker contains only glycine residues, e.g., at least 4 glycine residues (e.g., 4-200, 4-180, 4-160, 4-140, 4-40, 4-100, 4-90, 4-80, 4-70, 4-60, 4-50, 4-40, 4-30, 4-20, 4-19, 4-18, 4-17, 4-16, 4-15, 4-14, 4-13, 4-12, 4-11, 4-10, 4-9, 4-8, 4-7, 4-6 or 4-5 glycine residues) (e.g., 4-200, 6-200, 8-200, 10-200, 12-200, 14-200, 16-200, 18-200, 20-200, 30-200, 40-200, 50-200, 60-200, 70-200, 80-200, 90-200, 100-200, 120-200, 140-200, 160-200, 180-200, or 190-200 glycine residues). In some embodiments, a linker has 4-30 glycine residues (e.g., 4, 5, 6, 7, 8, 9, 10, 11, 12, 13, 14, 15, 16, 17, 18, 19, 20, 21, 22, 23, 24, 25, 26, 27, 28, 29, or 30 glycine residues). In some embodiments, a linker containing only glycine residues may not be glycosylated (e.g., O-linked glycosylation, also referred to as O-glycosylation) or may have a decreased level of glycosylation (e.g., a decreased level of O-glycosylation) (e.g., a decreased level of O-glycosylation with glycans such as xylose, mannose, sialic acids, fucose (Fuc), and/or galactose (Gal) (e.g., xylose)) as compared to, e.g., a linker containing one or more serine residues.

In some embodiments, a linker containing only glycine residues may not be O-glycosylated (e.g., O-xylosylation) or may have a decreased level of O-glycosylation (e.g., a decreased level of O-xylosylation) as compared to, e.g., a linker containing one or more serine residues.

In some embodiments, a linker containing only glycine residues may not undergo proteolysis or may have a decreased rate of proteolysis as compared to, e.g., a linker containing one or more serine residues.

In some embodiments, a linker can contain motifs of GGGG (SEQ ID NO: 78), e.g., GGGGGGGG (SEQ ID NO: 79), GGGGGGGGGGGG (SEQ ID NO: 80), GGGGGGGGGGGGGGGG (SEQ ID NO: 81), or GGGGGGGGGGGGGGGGGGGG (SEQ ID NO: 82). In some embodiments, a linker can contain motifs of GGGGG (SEQ ID NO: 83), e.g., GGGGGGGGGG (SEQ ID NO: 84), GGGGGGGGGGGGGGG (SEQ ID NO: 85), or GGGGGGGGGGGGGGGGGGGG (SEQ ID NO: 82). In some embodiments, a linker is GGGGGGGGGGGGGGGGGGGG (SEQ ID NO: 82).

In other embodiments, a linker can also contain amino acids other than glycine and serine, e.g., GENLYFQSGG (SEQ ID NO: 86), SACYCELS (SEQ ID NO: 87), RSIAT (SEQ ID NO: 88), RPACKIPNDLKQKVMNH (SEQ ID NO: 89), GGSAGGSGSGSSGGSSGASGTGTAGGTGSGSGTGSG (SEQ ID NO: 90), AAANSSIDLISVPVDSR (SEQ ID NO: 91), or

(SEQ ID NO: 92)
GGSGGGSEGGGSEGGGSEGGGSEGGGSEGGGSGGGS.

### Chemical Linkers

In some embodiments, a linker provides space, rigidity, and/or flexibility between the therapeutic agent and the variant Fc domain monomer or variant Fc domain in the conjugates and fusion proteins described here or between two therapeutic agents in the conjugates described herein. In some embodiments, a linker may be a bond, e.g., a covalent bond, e.g., an amide bond, a disulfide bond, a C—O bond, a C—N bond, a N—N bond, a C—S bond, or any kind of bond created from a chemical reaction, e.g., chemical conjugation. In some embodiments, a linker (L as shown in formula (1)) includes no more than 250 atoms (e.g., 1-2, 1-4, 1-6, 1-8, 1-10, 1-12, 1-14, 1-16, 1-18, 1-20, 1-25, 1-30, 1-35, 1-40, 1-45, 1-50, 1-55, 1-60, 1-65, 1-70, 1-75, 1-80, 1-85, 1-90, 1-95, 1-100, 1-110, 1-120, 1-130, 1-140, 1-150, 1-160, 1-170, 1-180, 1-190, 1-200, 1-210, 1-220, 1-230, 1-240, or 1-250 atom(s); 250, 240, 230, 220, 210, 200, 190, 180, 170, 160, 150, 140, 130, 120, 110, 100, 95, 90, 85, 80, 75, 70, 65, 60, 55, 50, 45, 40, 35, 30, 28, 26, 24, 22, 20, 18, 16, 14, 12, 10, 9, 8, 7, 6, 5, 4, 3, 2, or 1 atom(s)). In some embodiments, a linker (L) includes no more than 250 non-hydrogen atoms (e.g., 1-2, 1-4, 1-6, 1-8, 1-10, 1-12, 1-14, 1-16, 1-18, 1-20, 1-25, 1-30, 1-35, 1-40, 1-45, 1-50, 1-55, 1-60, 1-65, 1-70, 1-75, 1-80, 1-85, 1-90, 1-95, 1-100, 1-110, 1-120, 1-130, 1-140, 1-150, 1-160, 1-170, 1-180, 1-190, 1-200, 1-210, 1-220, 1-230, 1-240, or 1-250 non-hydrogen atom(s); 250, 240, 230, 220, 210, 200, 190, 180, 170, 160, 150, 140, 130, 120, 110, 100, 95, 90, 85, 80, 75, 70, 65, 60, 55, 50, 45, 40, 35, 30, 28, 26, 24, 22, 20, 18, 16, 14, 12, 10, 9, 8, 7, 6, 5, 4, 3, 2, or 1 non-hydrogen atom(s)). In some embodiments, the backbone of a linker (L) includes no more than 250 atoms (e.g., 1-2, 1-4, 1-6, 1-8, 1-10, 1-12, 1-14, 1-16, 1-18, 1-20, 1-25, 1-30, 1-35, 1-40, 1-45, 1-50, 1-55, 1-60, 1-65, 1-70, 1-75, 1-80, 1-85, 1-90, 1-95, 1-100, 1-110, 1-120, 1-130, 1-140, 1-150, 1-160, 1-170, 1-180, 1-190, 1-200, 1-210, 1-220, 1-230, 1-240, or 1-250 atom(s); 250, 240, 230, 220, 210, 200, 190, 180, 170, 160, 150, 140, 130, 120, 110, 100, 95, 90, 85, 80, 75, 70, 65, 60, 55, 50, 45, 40, 35, 30, 28, 26, 24, 22, 20, 18, 16, 14, 12, 10, 9, 8, 7, 6, 5, 4, 3, 2, or 1 atom(s)). The “backbone” of a linker refers to the atoms in the linker that together form the shortest path from one part of the conjugate to another part of the conjugate. The atoms in the backbone of the linker are directly involved in linking one part of the conjugate to another part of the conjugate. For examples, hydrogen atoms attached to carbons in the backbone of the linker are not considered as directly involved in linking one part of the conjugate to another part of the conjugate.

Molecules that may be used to make linkers (L) include at least two functional groups, e.g., two carboxylic acid groups. In some embodiments of a trivalent linker, two arms of a linker may contain two dicarboxylic acids, in which the first carboxylic acid may form a covalent linkage with the first therapeutic agent in the conjugate and the second carboxylic acid may form a covalent linkage with the second therapeutic agent in the conjugate, and the third arm of the linker may for a covalent linkage (e.g., a C—O bond) with a variant Fc domain monomer of variant Fc domain in the conjugate or fusion protein described herein. In some embodiments of a divalent linker, the divalent linker may contain two carboxylic acids, in which the first carboxylic acid may form a covalent linkage with one component (e.g., a therapeutic agent) in the conjugate and the second carboxylic acid may form a covalent linkage (e.g., a C—S bond or a C—N bond) with another component (e.g., a variant Fc domain monomer or variant Fc domain) in the conjugate.

In some embodiments, dicarboxylic acid molecules may be used as linkers (e.g., a dicarboxylic acid linker). For example, in a conjugate containing a variant Fc domain monomer or variant Fc domain covalently linked to one or more dimers of a therapeutic agent, the first carboxylic acid in a dicarboxylic acid molecule may form a covalent linkage with a hydroxyl or amine group of the first therapeutic agent and the second carboxylic acid may form a covalent linkage with a hydroxyl or amine group of the second therapeutic agent. In some instances, where a reactive group (e.g., carboxylic acid, hydroxyl, or amine) is not available on a therapeutic agent, a reactive group (e.g., a carboxylic acid, hydroxyl, or amine) can be introduced into the therapeutic agent in a way as to not disrupt the activity of the therapeutic agent.

In some embodiments, dicarboxylic acid molecules, such as the ones described herein, may be further functionalized to contain one or more additional functional groups. Dicarboxylic acids may be further functionalized, for example, to provide an attachment point to a variant Fc domain monomer, variant Fc domain, or fusion protein described herein (e.g., by way of a linker, such as a PEG linker).

In some embodiments, when the therapeutic agent is attached to a variant Fc domain monomer or variant Fc domain, the linking group may include a moiety including a carboxylic acid moiety and an amino moiety that are spaced by from 1 to 25 atoms.

In some embodiments, a linking group may include a moiety including a carboxylic acid moiety and an amino moiety, such as the ones described herein, may be further functionalized to contain one or more additional functional groups. Such linking groups may be further functionalized, for example, to provide an attachment point to a variant Fc domain monomer, variant Fc domain, or fusion protein described herein (e.g., by way of a linker, such as a PEG linker).

In some embodiments, when the therapeutic agent is attached to a variant Fc domain monomer or a variant Fc domain, the linking group may include a moiety including two or amino moieties (e.g., a diamino moiety) that are spaced by from 1 to 25 atoms.

In some embodiments, a linking group may include a diamino moiety, such as the ones described herein, may be further functionalized to contain one or more additional functional groups. Such diamino linking groups may be further functionalized, for example, to provide an attachment point to a variant Fc domain monomer, variant Fc domain, or fusion protein described herein (e.g., by way of a linker, such as a PEG linker).

In some embodiments, a molecule containing an azide group may be used to form a linker, in which the azide group may undergo cycloaddition with an alkyne to form a 1,2,3-triazole linkage. In some embodiments, a molecule containing an alkyne group may be used to form a linker, in which the alkyne group may undergo cycloaddition with an azide to form a 1,2,3-triazole linkage. In some embodiments, a molecule containing a maleimide group may be used to form a linker, in which the maleimide group may react with a cysteine to form a C—S linkage. In some embodiments, a molecule containing one or more sulfonic acid groups may be used to form a linker, in which the sulfonic acid group may form a sulfonamide linkage with a linking nitrogen in a therapeutic agent. In some embodiments, a molecule containing one or more isocyanate groups may be used to form a linker, in which the isocyanate group may form a urea linkage with a linking nitrogen in a therapeutic agent. In some embodiments, a molecule containing one or more haloalkyl groups may be used to form a linker, in which the haloalkyl group may form a covalent linkage, e.g., C—N and C—O linkages, with a therapeutic agent.

In some embodiments, a linker (L) may include a synthetic group derived from, e.g., a synthetic polymer (e.g., a polyethylene glycol (PEG) polymer). In some embodiments, a linker may include one or more amino acid residues. In some embodiments, a linker may be an amino acid sequence (e.g., a 1-25 amino acid, 1-10 amino acid, 1-9 amino acid, 1-8 amino acid, 1-7 amino acid, 1-6 amino acid, 1-5 amino acid, 1-4 amino acid, 1-3 amino acid, 1-2 amino acid, or 1 amino acid sequence). In some embodiments, a linker (L) may include one or more optionally substituted C1-C20 alkylene, optionally substituted C1-C20 heteroalkylene (e.g., a PEG unit), optionally substituted C2-C20 alkenylene (e.g., C2 alkenylene), optionally substituted C2-C20 heteroalkenylene, optionally substituted C2-C20 alkynylene, optionally substituted C2-C20 heteroalkynylene, optionally substituted C3-C20 cycloalkylene (e.g., cyclopropylene, cyclobutylene), optionally substituted C3-C20 heterocycloalkylene, optionally substituted C4-C20 cycloalkenylene, optionally substituted C4-C20 heterocycloalkenylene, optionally substituted C8-C20 cycloalkynylene, optionally substituted C8-C20 heterocycloalkynylene, optionally substituted C5-C15 arylene (e.g., C6 arylene), optionally substituted C2-C15 heteroarylene (e.g., imidazole, pyridine), O, S, NRⁱ (Rⁱ is H, optionally substituted C1-C20 alkyl, optionally substituted C1-C20 heteroalkyl, optionally substituted C2-C20 alkenyl, optionally substituted C2-C20 heteroalkenyl, optionally substituted C2-C20 alkynyl, optionally substituted C2-C20 heteroalkynyl, optionally substituted C3-C20 cycloalkyl, optionally substituted C3-C20 heterocycloalkyl, optionally substituted C4-C20 cycloalkenyl, optionally substituted C4-C20 heterocycloalkenyl, optionally substituted C8-C20 cycloalkynyl, optionally substituted C8-C20 heterocycloalkynyl, optionally substituted C5-C15 aryl, or optionally substituted C2-C15 heteroaryl), P, carbonyl, thiocarbonyl, sulfonyl, phosphate, phosphoryl, or imino.

### Conjugation Chemistries

Covalent conjugation of two or more components in a conjugate using a linker may be accomplished using well-known organic chemical synthesis techniques and methods. Complementary functional groups on two components may react with each other to form a covalent bond. Examples of complementary reactive functional groups include, but are not limited to, e.g., maleimide and cysteine, amine and activated carboxylic acid, thiol and maleimide, activated sulfonic acid and amine, isocyanate and amine, azide and alkyne, and alkene and tetrazine. Site-specific conjugation to a polypeptide (e.g., a variant Fc domain monomer, a variant Fc domain, or a fusion protein) may be accomplished using techniques known in the art. Exemplary techniques for site-specific conjugation of a small molecule to an Fc domain monomer of an Fc domain (e.g., a variant Fc domain monomer or variant Fc domain described here) are provided in Agarwall. P., et al. Bioconjugate Chem. 26:176-192 (2015).

Other examples of functional groups capable of reacting with amino groups include, e.g., alkylating and acylating agents. Representative alkylating agents include: (i) an α-haloacetyl group, e.g., XCH₂CO— (where X═Br, Cl, or I); (ii) a N-maleimide group, which may react with amino groups either through a Michael type reaction or through acylation by addition to the ring carbonyl group; (iii) an aryl halide, e.g., a nitrohaloaromatic group; (iv) an alkyl halide; (v) an aldehyde or ketone capable of Schiff's base formation with amino groups; (vi) an epoxide, e.g., an epichlorohydrin and a bisoxirane, which may react with amino, sulfhydryl, or phenolic hydroxyl groups; (vii) a chlorine-containing of s-triazine, which is reactive towards nucleophiles such as amino, sulfhydryl, and hydroxyl groups; (viii) an aziridine, which is reactive towards nucleophiles such as amino groups by ring opening; (ix) a squaric acid diethyl ester; and (x) an α-haloalkyl ether.

Examples of amino-reactive acylating groups include, e.g., (i) an isocyanate and an isothiocyanate; (ii) a sulfonyl chloride; (iii) an acid halide; (iv) an active ester, e.g., a nitrophenylester or N-hydroxysuccinimidyl ester, or derivatives thereof (e.g., azido-PEG₂-PEG₄₀-NHS ester); (v) an acid anhydride, e.g., a mixed, symmetrical, or N-carboxyanhydride; (vi) an acylazide; and (vii) an imidoester. Aldehydes and ketones may be reacted with amines to form Schiff's bases, which may be stabilized through reductive amination.

It will be appreciated that certain functional groups may be converted to other functional groups prior to reaction, for example, to confer additional reactivity or selectivity. Examples of methods useful for this purpose include conversion of amines to carboxyls using reagents such as dicarboxylic anhydrides; conversion of amines to thiols using reagents such as N-acetylhomocysteine thiolactone, S-acetylmercaptosuccinic anhydride, 2-iminothiolane, or thiol-containing succinimidyl derivatives; conversion of thiols to carboxyls using reagents such as α-haloacetates; conversion of thiols to amines using reagents such as ethylenimine or 2-bromoethylamine; conversion of carboxyls to amines using reagents such as carbodiimides followed by diamines; and conversion of alcohols to thiols using reagents such as tosyl chloride followed by transesterification with thioacetate and hydrolysis to the thiol with sodium acetate.

In some embodiments, a linker of the invention (e.g., L), is conjugated (e.g., by any of the methods described herein) to a variant Fc domain monomer (e.g., E). In preferred embodiments of the invention, the linker is conjugated by way of: (a) a thiourea linkage (i.e., —NH(C═S)NH—) to a lysine of E; (b) a carbamate linkage (i.e., —NH(C═O)—O) to a lysine of E; (c) an amine linkage by reductive amination (i.e., —NHCH₂) between a lysine and E; (d) an amide (i.e., —NH—(C═O)CH₂) to a lysine of E; (e) a cysteine-maleimide conjugate between a maleimide of the linker to a cysteine of E; (f) an amine linkage by reductive amination (i.e., —NHCH₂) between the linker and a carbohydrate of E (e.g., a glycosyl group of a variant Fc domain monomer or a variant Fc domain); (g) a rebridged cysteine conjugate, wherein the linker is conjugated to two cysteines of E; (h) an oxime linkage between the linker and a carbohydrate of E (e.g., a glycosyl group of a variant Fc domain monomer or a variant Fc domain); (i) an oxime linkage between the linker and an amino acid residue of E; (j) an azido linkage between the linker and E; (k) direct acylation of a linker to E; or (l) a thioether linkage between the linker and E.

In some embodiments, a linker is conjugated to E, wherein the linkage includes the structure —NH(C═NH)X—, wherein X is O, HN, or a bond. In some embodiments, a linker is conjugated to E, wherein the linkage between the remainder of the linker and E includes the structure —NH(C═O)NH—.

In some embodiments, a linker (e.g., an active ester, e.g., a nitrophenylester or N-hydroxysuccinimidyl ester, or derivatives thereof (e.g., a functionalized PEG linker (e.g., azido-PEG₂-PEG₄₀-NHS ester), is conjugated to E, with a T of (e.g., DAR) of between 0.5 and 10.0, e.g., 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8.0, 7.9, 8, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10.0. In these instances, the E-(PEG₂-PEG₄₀)-azide can react with a modified therapeutic agent having a terminal alkyne linker (e.g., L) through click conjugation. During click conjugation, the copper-catalyzed reaction of the an azide (e.g., the Fc-(PEG₂-PEG₄₀)-azide) with the alkyne (e.g., the modified therapeutic agent having a terminal alkyne linker (e.g., L) forming a 5-membered heteroatom ring. In some embodiments, the linker conjugated to E is a terminal alkyne and is conjugated to a modified therapeutic agent having a terminal azide. One of skill in the art would readily understand the final product from a click chemistry conjugation.

### V. Methods

Methods described herein include, e.g., methods of protecting against or treating a condition or disorder described herein (e.g., a respiratory disorder, a hepatic disorder, a central nervous system disorder, a muscular disorder, a skin disorder, an ocular disorder, a vascular disorder, or an infection (e.g., a viral infection, a fungal infection, or a bacterial infection)) in a subject and methods of preventing, stabilizing, or inhibiting the growth of infection pathogens (e.g., viral particles, fungi, or bacterium). A method of treating a condition or disorder described herein (e.g., a respiratory disorder, a hepatic disorder, a central nervous system disorder, a muscular disorder, a skin disorder, an ocular disorder, a vascular disorder, or an infection (e.g., a viral infection, a fungal infection, or a bacterial infection)) in a subject includes administering to the subject a conjugate described herein (e.g., a conjugate of formula (1)), fusion protein described herein, or a pharmaceutical composition thereof.

Viral Infections

The compounds and pharmaceutical compositions described herein (e.g., a conjugate of formula (1) or a fusion protein described herein) can be used to treat a viral infection (e.g., viral meningitis, herpes simplex virus (HSV) 1, HSV 2, Epstein-Barr virus, varicella-zoster virus, poliovirus, coxsackievirus, West Nile virus, Lacrosse virus, western equine encephalitis, eastern equine encephalitis, Powassan virus, rabies virus, respiratory syncytial virus (RSV), dengue, a beta coronavirus (e.g., COVID-19), zika virus, or an influenza viral infection, such as influenza A, B, C, or parainfluenza).

Viral infection refers to the pathogenic growth of a virus in a host organism (e.g., a human subject). A viral infection can be any situation in which the presence of a viral population(s) is damaging to a host body. Thus, a subject is suffering from a viral infection when an excessive amount of a viral population is present in or on the subject's body, or when the presence of a viral population(s) is damaging the cells or other tissue of the subject.

Influenza, commonly known as “the flu”, is an infectious disease caused by an influenza virus. Symptoms can be mild to severe. The most common symptoms include: a high fever, runny nose, sore throat, muscle pains, headache, coughing, and feeling tired. These symptoms typically begin two days after exposure to the virus and most last less than a week. The cough, however, may last for more than two weeks. In children, there may be nausea and vomiting, but these are less common in adults. Complications of influenza may include viral pneumonia, secondary bacterial pneumonia, sinus infections, and worsening of previous health problems such as asthma or heart failure. Sever complications may occur in subjects having weakened immune systems, such as the young, the old, those with illnesses that weaken the immune system, and those undergoing therapy treatment resulting in a weakening of the immune system.

Three types of influenza viruses affect human subjects, namely Type A, Type B, and Type C. Usually, the virus is spread through the air from coughs or sneezes. This is believed to occur mostly over relatively short distances. It can also be spread by touching surfaces contaminated by the virus and then touching the mouth or eyes. A person may be infectious to others both before and during the time they are showing symptoms. The infection may be confirmed by testing the throat, sputum, or nose for the virus. A number of rapid tests are available; however, people may still have the infection if the results are negative. A type of polymerase chain reaction that detects the virus's RNA may be used to diagnose influenza infection.

Viral infection may refer to the pathogenic growth of a virus (e.g., RSV such as RSV A or RSV B) in a host organism (e.g., a human subject). Human respiratory syncytial virus (RSV) is a medium-sized (120-200 nm) enveloped virus that contains a lipoprotein coat and a linear negative-sense RNA genome (must be converted to a positive RNA prior to translation). The former contains virally encoded F, G, and SH lipoproteins. The F and G lipoproteins are the only two that target the cell membrane, and are highly conserved among RSV isolates. Human RSV (HRSV) is divided into two antigenic subgroups, A and B, on the basis of the reactivity of the virus with monoclonal antibodies against the attachment (G) and fusion (F) glycoproteins. Subtype B is characterized as the asymptomatic strains of the virus that the majority of the population experiences. The more severe clinical illnesses involve subtype A strains, which tend to predominate in most outbreaks.

Four of the viral genes code for intracellular proteins that are involved in genome transcription, replication, and particle budding, namely N (nucleoprotein), P (phosphoprotein), M (matrix protein), and L (“large” protein, containing the RNA polymerase catalytic motifs). The RSV genomic RNA forms a helical ribonucleoprotein (RNP) complex with the N protein, termed nucleocapsid, which is used as template for RNA synthesis by the viral polymerase complex. The three-dimensional crystal structure of a decameric, annular ribonucleoprotein complex of the RSV nucleoprotein (N) bound to RNA has been determined at 3.3 Å resolution. This complex mimics one turn of the viral helical nucleocapsid complex. Its crystal structure was combined with electron microscopy data to provide a detailed model for the RSV nucleocapsid

Viral infection may refer to Aseptic meningitis (AM) is defined as an inflammation of the subarachnoid space, characterized by mononuclear cells pleocytosis and by sterile CSF (cerebrospinal fluid or cerebrospinal fluid) culture. The primary cause of AMs are viral infections (Ravel R: Clinical Laboratory Medicine: Clinical Application of Laboratory Data: Elsevier Health Sciences; 1994). Viral meningitis are common and often not reported. Non-poliovirus enteroviruses (Coxsackievirus and Echovirus) are responsible for 80 to 90% of the cases of viral meningitis with determined etiology (Atkinson P, Sharland M, Maguire H: Predominant enteroviral serotypes causing meningitis. Archives of Disease in Childhood 1998, 78:373-374).

Viral infection may refer to herpes simplex virus 1 (HSV 1) or HSV 2. HSV 1 is the usual cause of cold sores on the lips (herpes labialis) and sores on the cornea of the eye (herpes simplex keratitis). HSV 2 is the usual cause of genital herpes. The distinction between the two is not absolute. Genital infections are sometimes caused by HSV 1. Infection can also occur in other parts of the body such as the brain (a serious illness) or gastrointestinal tract. Widespread infection may occur in newborns or in people with a weakened immune system, particularly those with an HIV infection. HSV is very contagious and can spread by direct contact with sores and sometimes by contact with the mouth or genitals of people who have HSV infection even when no sores can be seen.

Viral infection may refer to Coxsackievirus. Coxsackievirus are a few related enteroviruses that belong to the Picornaviridae family of nonenveloped, linear, positive-sense single-stranded RNA viruses, as well as its genus Enterovirus, which also includes poliovirus and echovirus. Coxsackievirus, while being among the leading cause of aseptic meningitis, may cause hand, foot, and mouth disease, as well as disease of muscles, lungs, and heart.

The invention also provides a method of preventing, stabilizing, or inhibiting the growth of viral particles or preventing the replication and spread of the virus includes contacting the virus or a site susceptible to viral growth with a conjugate described herein (e.g., a conjugate of any one of formula (1)), a fusion protein described herein, or a pharmaceutical composition thereof. In some embodiments, the virus is a resistant strain of a virus.

Moreover, methods described herein also include methods of protecting against or treating viral infection in a subject by administering to the subject a composition described herein (e.g., a conjugate of formula (1)) or fusion protein described herein in combination with a second therapeutic, such as an antiviral agent or an antiviral vaccine.

Bacterial Infections

The compounds and pharmaceutical compositions described herein (e.g., a conjugate of formula (1) or a fusion protein described herein) can be used to treat a bacterial infection.

Bacterial infection refers to the pathogenic growth of bacteria (e.g., Acinetobacter spp. (Acinetobacter baumanni), Bacteroides distasonis, Bacteroides fragilis, Bacteroides ovatus, Bacteroides thetaiotaomicron, Bacteroides uniformis, Bacteroides vulgatus, Citrobacter freundii, Citrobacter koser, Clostridium clostridioforme, Clostridium perfringens, Enterobacter aerogenes, Enterobacter cloacae, Enterococcus faecalis, Enterococcus spp. (vancomycin susceptible and resistant isolates), Escherichia coli (including ESBL and KPC producing isolates), Eubacterium lentum, Fusobacterium spp., Haemophilus influenzae (including beta-lactamase positive isolates), Haemophilus parainfluenzae, Klebsiella pneumoniae (including ESBL and KPC producing isolates), Klebsiella oxytoca (including ESBL and KPC producing isolates), Legionella pneumophilia Moraxella catarrhalis, Morganella morganii, Mycoplasma spp., Peptostreptococcus spp., Porphyromonas asaccharolytica, Prevotella bivia, Proteus mirabilis, Proteus vulgaris, Providencia rettgeri, Providencia stuartii, Pseudomonas aeruginosa, Serratia marcescens, Streptococcus anginosus, Staphylococcus aureus (methicillin susceptible and resistant isolates), Staphylococcus epidermidis (methicillin susceptible and resistant isolates), Stenotrophomonas maltophilia, Streptococcus agalactiae, Streptococcus constellatus, Streptococcus pneumoniae (penicillin susceptible and resistant isolates), and Streptococcus pyogenes) in a host organism (e.g., a human subject). A bacterial infection can be any situation in which the presence of a bacterial population(s) is damaging to a host body. Thus, a subject is suffering from a bacterial infection when an excessive amount of a bacteria population is present in or on the subject's body, or when the presence of a bacterial population(s) is damaging the cells or other tissue of the subject.

Staphylococcus aureus is a major human pathogen, and it is estimated that approximately 30% of humans are asymptomatic nasal carriers (Chambers and DeLeo 2009. Nat. Rev. Microbiol. 7:629-641). S. aureus causes skin, soft tissue, respiratory, bone, joint and endovascular diseases. Life threatening cases caused by S. aureus include bacteremia, endocarditis, sepsis and toxic shock syndrome (Lowy 1998. N. Engl. J. Med. 339:520-532). Antibiotic resistance in S. aureus is increasingly becoming an urgent medical problem. The methicillin resistance in S. aureus is approaching epidemic level (Chambers and DeLeo, supra; Grundmann et al., 2006. Lancet 368:874-885). It was estimated that 94,360 invasive MRSA infections occurred in the US in 2005, and these infections were associated with death in 18,650 cases (Klevens et al., 2007. JAMA 298:1763-1771). Although S. epidermidis is part of the normal human epithelial bacterial flora, it can cause infection when skin or mucous membrane is injured.

Exemplary therapeutic agents that are effective against multiplying bacteria and thus can be conjugated to Fc variants of the invention are β-lactams such as penicillins (e.g., penicillin G, penicillin V, methicillin, oxacillin, cloxacillin, dicloxacillin, nafcillin, ampicillin, amoxicillin, carbenicillin, ticarcillin, mezlocillin, piperacillin, azlocillin, and temocillin), cephalosporins (e.g., cepalothin, cephapirin, cephradine, cephaloridine, cefazolin, cefamandole, cefuroxime, cephalexin, cefprozil, cefaclor, loracarbef, cefoxitin, cefmetazole, cefotaxime, ceftizoxime, ceftriaxone, cefoperazone, ceftazidime, cefixime, cefpodoxime, ceftibuten, cefdinir, cefpirome, cefepime, BAL5788, and BAL9141), carbapenems (e.g., imipenem, ertapenem, and meropenem), and monobactams (e.g., aztreonam); β-lactamase inhibitors (e.g., clavulanate, sulbactam, and tazobactam); aminoglycosides (e.g., streptomycin, neomycin, kanamycin, paromomycin, puromycin, gentamicin, tobramycin, amikacin, netilmicin, spectinomycin, sisomicin, dibekalin, and isepamicin); tetracyclines (e.g., tetracycline, chlortetracycline, demeclocycline, minocycline, oxytetracycline, methacycline, and doxycycline); macrolides (e.g., erythromycin, azithromycin, and clarithromycin); ketolides (e.g., telithromycin, ABT-773); lincosamides (e.g., lincomycin and clindamycin); glycopeptides (e.g., vancomycin, oritavancin, dalbavancin, and teicoplanin); streptogramins (e.g., quinupristin and dalfopristin); sulphonamides (e.g., sulphanilamide, para-aminobenzoic acid, sulfadiazine, sulfisoxazole, sulfamethoxazole, and sulfathalidine); oxazolidinones (e.g., linezolid); quinolones (e.g., nalidixic acid, oxolinic acid, norfloxacin, pefloxacin, enoxacin, ofloxacin, ciprofloxacin, temafloxacin, lomefloxacin, fleroxacin, grepafloxacin, sparfloxacin, trovafloxacin, clinafloxacin, gatifloxacin, moxifloxacin, gemifloxacin, and sitafloxacin); metronidazole; daptomycin; garenoxacin; ramoplanin; faropenem; polymyxin; tigecycline, AZD2563; and trimethoprim.

Methods described herein include, e.g., methods of protecting against or treating an infection (e.g., a bacterial infection) in a subject and methods of preventing, stabilizing, or inhibiting the growth of infection pathogens (e.g., bacterium). A method of treating an infection (e.g., a bacterial infection) in a subject includes administering to the subject a conjugate described herein (e.g., a conjugate of formula (1)), a fusion protein described herein, or a pharmaceutical composition thereof. In some embodiments, the bacterial infection is caused by a resistant strain of bacteria. A method of preventing, stabilizing, or inhibiting the growth of bacteria or preventing the replication and spread of the bacteria includes contacting the bacteria or a site susceptible to bacterial growth with a conjugate described herein (e.g., a conjugate of any one of formulas (1)) or a pharmaceutical composition thereof.

Moreover, methods described herein also include methods of protecting against or treating bacterial infection in a subject by administering to the subject a conjugate described herein (e.g., a conjugate of formula (1)) or fusion protein described herein in combination with a second therapeutic agent, such as an antibacterial agent.

Fungal Infections

The compounds and pharmaceutical compositions described herein (e.g., a conjugate of formula (1) or a fusion protein described herein) can be used to treat a fungal infection.

Fungal infection refers to the pathogenic growth of a fungus (e.g., Trichophyton species (e.g., T. ajelloi, T. concentricum, T. equinum, T. erinacei, T. flavescens, T. gloriae, T. interdigitale, T. megnini, T. mentagrophytes, T. phaseoliforme, T. rubrum, T. schoenleini, T. simii, T. soudanense, T. terrestre, T. tonsurans, T. vanbreuseghemii, T. verrucosum, T. violaceum, or T. yaoundei), Epidermophyton species (e.g., E. floccosum or E. stockdaleae), Candida species (e.g., C. albicans, C. parapsiliosis, C. krusei, C. tropicalis, C. glabrata, C. parapsilosis, C. lusitaniae, C. kefyr, C. guilliermondii, or C. dubliniensis), Microsporum species (e.g., M. canis, M. gypseum, M. audouini, M. gallinae, M. ferrugineum, M. distortum, M. nanum, M. cookie, or M. vanbreuseghemii), Epicoccum species (e.g., E. nigrum), Aspergillus species (e.g., A. sydowii, A. terreus, A. niger, A. terreus, A. fumigatus, A. flavus, A. clavatus, A. glaucus group, A. nidulans, A. oryzae, A. terreus, A. ustus, or A. versicolor), Paecilomyces species (e.g., P. lilacinus or P. variotil), Fusarium species (e.g., F. oxysporum, F. solani, or F. semitectum), Acremonium species (e.g., A. strictum, A. roseogiseum, A. cucurbitacearum, A. kiliense, A. curvatum, A. comptosporum, Ulocladium chartarum, A. alternatum, or Emercellopsis minima), Chaetomium species (e.g., C. atrobrunneum, C. funicola, C. globosum, or C. strumarium), Phoma species, Scopulariopsis species (e.g., S. brevicaulis, S. candida, S. koningii, S. acremonium, S. flava, S. cinerea, S. trigonospora, S. brumptii, S. chartarum, S. fusca, or S. asperula), Alternaria species (e.g., A. alternate, A. chartarum, A. dianthicola, A. geophilia, A. infectoria, A. stemphyloides, or A. teunissima), and Curvularia species (e.g., C. brachyspora, C. clavata, C. geniculata, C. lunata, C. pallescens, C. senegalensis, or C. verruculosa) in a host organism (e.g., a human subject). A fungal infection can be any situation in which the presence of a fungal population(s) is damaging to a host body. Thus, a subject is suffering from a fungal infection when an excessive amount of a fungal population is present in or on the subject's body, or when the presence of a fungal population(s) is damaging the cells or other tissue of the subject.

Fungi cause a wide variety of diseases in humans. While some fungi cause infections limited to the outermost layers of the skin and hair (superficial mycoses), other fungi cause cutaneous mycoses by penetrating to the keratinized layers of the skin, hair, and nails and triggering pathologic changes in the host. Subcutaneous mycoses cause infections in the dermis, subcutaneous tissues, muscle, and fascia and are often chronic. Systemic mycoses originate primarily in the lung and may cause secondary infections in other organ systems in the body. Patients with immune system deficiencies are often prone to opportunistic mycoses.

Dermatophytes, including Trichophyton rubrum and Trichophyton mentagrophytes, are responsible for fungal infections of the skin or Dermatophytoses (dermatophytose). Tinea pedis is a skin infection that most often manifests between the toes, causing scaling, flaking, and itching of the affected skin. Blisters and cracked skin may also occur, leading to exposed raw tissue, erythema, pain, swelling and inflammation. A second type of tinea pedis is called the moccasin tinea pedis and is characterized by chronic plantar erythema with slight scaling to diffuse hyperkeratosis that can be asymptomatic or pruritic (e.g., uncomfortable, irritating sensation). Other types include inflammatory/vesicular and ulcerative tinea pedis. The infection can be spread to other areas of the body, and manifest itself in the form of annular scaly plaques with raised edges, pustules, and vesicles in the trunk and arms and legs (Tinea corporis), scaly rash in the palms and finger webs (Tinea manuum), erythematous lesions in the groin and pubic region (Tinea cruris), erythema, scaling, and pustules in the beard and neck area (Tinea barbae or Tinea faciale), or round, bald, scaly patches in the scalp (Tinea capitis). Tinea versicolor, also called pityriasis versicolor, is a common fungal infection of the skin that interferes with the normal pigmentation of the skin, resulting in small, discolored patches. Tinea unguium is another term for dermatophyte infections of the nail. Secondary bacterial infections may develop from the fungal infection.

Tinea is very common, especially among children, and may be spread by skin-to-skin contact, as well as via contact with contaminated items such as hairbrushes or through the use of the same toilet seat as an infected individual. Tinea spreads readily, as those infected are contagious even before they show symptoms of the disease. Participants in contact sports such as wrestling have a risk of contracting the fungal infection through skin-to-skin contact.

Tinea is mildly contagious. Tinea is also a common infection in domestic animals, especially farm animals, dogs and cats and even small pets like hamsters or guinea pigs. Humans can contract tinea (also commonly referred to as “ringworm”) from these animals as humans are in close contact with them. Tinea can also be caught from other humans, both by direct contact and by prolonged contact with flakes of shed skin (from sharing clothes or from house dust, for instance).

The best known sign of tinea in people is the appearance of one or more red raised itchy patches with defined edges, not unlike the herald rash of Pityriasis rosea. These patches are often lighter in the center, taking on the appearance of a ring with hyperpigmentation around the circumference caused by an increase in melanin. If the infected area involves the scalp or beard area, then bald patches may become evident. The affected area may become itchy for periods of time.

Sometimes a tinea infection may cause skin lesions in a part of the body that is remote from the actual infection. Such lesions are called “dermatophytids”. The lesions themselves are fungus-free, and normally disappear upon treatment of the actual infection. The most common example is an eruption in the hands resulting from a fungus infection of the feet. Dermatophytids are essentially a generalized allergic reaction to the fungus.

Thus, fungi and yeast such as Microsporum species, Trichophyton species, Epidermophyton species, and Candida species can cause persistent and difficult to treat infections.

Microsporum species include M. canis and M. gypseum. Microsporum is one of the several fungal genera that cause dermatophytosis. Dermatophytosis is a general term used to define the infection in hair, skin, or nails due to any dermatophyte species. Similar to other dermatophytes, Microsporum has the ability to degrade keratin and thus can reside on skin and its appendages and remains noninvasive. Notably, Microsporum spp. mostly infect the hair and skin. Microsporum canis is the principal cause of ringworm in dogs and cats and a zoophilic fungal species causing sporadic dermatophytosis in humans, especially tinea capitis in children with cats and dogs.

Skin infection by a Trichophyton species occurs mainly on the back of the neck, scalp or beard. Symptoms of a Trichophyton species infection include inflamed scalp lesions, inflamed neck lesions, inflamed beard lesions, scarring, and permanent hair loss. Examples of Trichophyton species include T. rubrum, T. tonsurans and T. mentagrophytes.

Trichophyton tonsurans is an anthropophilic endothrix species of fungi that causes epidemic dermatophytosis in Europe, South America, and the U.S. It infects some animals and requires thiamine for growth. It is the most common cause of tinea capitis in the U.S., forming black dots where hair breaks off at the skin surface. Trichophyton rubrum is a fungus that is the most common cause of tinea pedis (“athlete's foot”), tinea cruris, and tinea (ringworm). Trichophyton rubrum is the most common of the dermatophytes causing fingernail fungus infections. While most fungal skin infections are irritating and difficult to treat, there are reports of fungal infections resulting in death. Specifically, a Trichophyton mentagrophytes skin infection migrated to the lymph nodes, testes, vertebrae and CNS. Treatment with griseofulvin, amphotericin B, clotrimazole, and transfer factor failed, eventually resulting in death of the subject (Hironaga et al., J. Clin. Microbiol., 2003; 5298-5301.) Trichophyton mentagrophytes is the second most common source of fungal nail infections from the dermatophyte group.

The genus Epidermophyton contains two species; Epidermophyton floccosum and Epidermophyton stockdaleae. E. stockdaleae is known to be nonpathogenic, leaving E. floccosum as the only species causing infections in humans. E. floccosum is one of the common causes of dermatophytosis in otherwise healthy individuals. It infects skin (tinea corporis, tinea cruris, tinea pedis) and nails (onychomycosis). The infection is restricted to the nonliving cornified layers of epidermis since the fungus lacks the ability to penetrate the viable tissues of the immunocompetent host. Disseminated infections due to any of the dermatophytes are very unlikely due to the restriction of the infection to keratinized tissues.

However, invasive E. floccosum infection has been reported in an immunocompromised patient with Behcet's syndrome. As with all forms of dermatophytosis, Epidermophyton floccosum infections are communicable and usually transmitted by contact, particularly in common showers and gym facilities.

Candida species include C. albicans, C. parapsiliosis, and C. krusei. Patients with chronic mucocutaneous candidiasis may develop candida infection of the nails. Candida species may invade nails previously damaged by infection or trauma and cause infection in the periungual area and underneath the nailbed. The nailfold becomes erythematous, swollen and tender with an occasional discharge. The disease causes loss of the cuticle, nail dystrophy, and onycholysis with discoloration around the lateral nailfold. In all forms of onychomycosis, the nail becomes variously disfigured and distorted.

Methods described herein also include methods of protecting against or treating fungal infection in a subject by administering to the subject a composition described herein (e.g., a conjugate of formula (1)) or fusion protein described herein in combination with an antifungal agent.

### VI. Pharmaceutical Compositions

A composition comprising a variant Fc domain (e.g., a conjugate or fusion protein described herein) may be formulated in a pharmaceutical composition for use in the methods described herein. In some embodiments, a conjugate or fusion protein described herein may be formulated in a pharmaceutical composition alone. In some embodiments, a conjugate or fusion protein described herein may be formulated in combination with an antiviral agent, antiviral vaccine, antifungal agent, antibacterial agent, or a therapeutic agent for the treatment of a disorder in a pharmaceutical composition. In some embodiments, the pharmaceutical composition includes a conjugate described herein (e.g., a conjugate described by formula (1)) or a fusion protein described herein and pharmaceutically acceptable carriers and excipients.

Acceptable carriers and excipients in the pharmaceutical compositions are nontoxic to recipients at the dosages and concentrations employed. Acceptable carriers and excipients may include buffers such as phosphate, citrate, HEPES, and TAE, antioxidants such as ascorbic acid and methionine, preservatives such as hexamethonium chloride, octadecyldimethylbenzyl ammonium chloride, resorcinol, and benzalkonium chloride, proteins such as human serum albumin, gelatin, dextran, and immunoglobulins, hydrophilic polymers such as polyvinylpyrrolidone, amino acid residues such as glycine, glutamine, histidine, and lysine, and carbohydrates such as glucose, mannose, sucrose, and sorbitol.

Examples of other excipients include, but are not limited to, antiadherents, binders, coatings, compression aids, disintegrants, dyes, emollients, emulsifiers, fillers (diluents), film formers or coatings, flavors, fragrances, glidants (flow enhancers), lubricants, sorbents, suspensing or dispersing agents, or sweeteners. Exemplary excipients include, but are not limited to: butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, stearic acid, sucrose, talc, titanium dioxide, vitamin A, vitamin E, vitamin C, and xylitol.

The conjugates or fusion proteins described herein may have ionizable groups so as to be capable of preparation as pharmaceutically acceptable salts. These salts may be acid addition salts involving inorganic or organic acids or the salts may, in the case of acidic forms of the conjugates herein be prepared from inorganic or organic bases. Frequently, the conjugates or fusion proteins are prepared or used as pharmaceutically acceptable salts prepared as addition products of pharmaceutically acceptable acids or bases. Suitable pharmaceutically acceptable acids and bases are well-known in the art, such as hydrochloric, sulphuric, hydrobromic, acetic, lactic, citric, or tartaric acids for forming acid addition salts, and potassium hydroxide, sodium hydroxide, ammonium hydroxide, caffeine, various amines, and the like for forming basic salts. Methods for preparation of the appropriate salts are well-established in the art.

Representative acid addition salts include, but are not limited to, acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, and valerate salts. Representative alkali or alkaline earth metal salts include, but are not limited to, sodium, lithium, potassium, calcium, and magnesium, as well as nontoxic ammonium, quaternary ammonium, and amine cations, including, but not limited to ammonium, tetramethylammonium, tetraethylammonium, methylamine, dimethylamine, trimethylamine, triethylamine, and ethylamine.

Depending on the route of administration and the dosage, a conjugate herein or a pharmaceutical composition thereof used in the methods described herein will be formulated into suitable pharmaceutical compositions to permit facile delivery. A conjugate (e.g., a conjugate of formula (1)) or a pharmaceutical composition thereof may be formulated to be administered intramuscularly, intravenously (e.g., as a sterile solution and in a solvent system suitable for intravenous use), intradermally, intraarterially, intraperitoneally, intralesionally, intracranially, intraarticularly, intraprostatically, intrapleurally, intratracheally, intranasally, intravitreally, intravaginally, intrarectally, topically, intratumorally, peritoneally, subcutaneously, subconjunctival, intravesicularlly, mucosally, intrapericardially, intraumbilically, intraocularally, orally (e.g., a tablet, capsule, caplet, gelcap, or syrup), topically (e.g., as a cream, gel, lotion, or ointment), locally, by inhalation, by injection, or by infusion (e.g., continuous infusion, localized perfusion bathing target cells directly, catheter, lavage, in cremes, or lipid compositions). Depending on the route of administration, a conjugate herein or a pharmaceutical composition thereof may be in the form of, e.g., tablets, capsules, pills, powders, granulates, suspensions, emulsions, solutions, gels including hydrogels, pastes, ointments, creams, plasters, drenches, osmotic delivery devices, suppositories, enemas, injectables, implants, sprays, preparations suitable for iontophoretic delivery, or aerosols. The compositions may be formulated according to conventional pharmaceutical practice.

A composition described herein may be formulated in a variety of ways that are known in the art. For use as treatment of human and animal subjects, a conjugate described herein can be formulated as pharmaceutical or veterinary compositions. Depending on the subject (e.g., a human) to be treated, the mode of administration, and the type of treatment desired, e.g., prophylaxis or therapy, a conjugate described herein is formulated in ways consonant with these parameters. A summary of such techniques is found in Remington: The Science and Practice of Pharmacy, 22nd Edition, Lippincott Williams & Wilkins (2012); and Encyclopedia of Pharmaceutical Technology, 4th Edition, J. Swarbrick and J. C. Boylan, Marcel Dekker, New York (2013), each of which is incorporated herein by reference.

Formulations may be prepared in a manner suitable for systemic administration or topical or local administration. Systemic formulations include those designed for injection (e.g., intramuscular, intravenous, or subcutaneous injection) or may be prepared for transdermal, transmucosal, or oral administration. The formulation will generally include a diluent as well as, in some cases, adjuvants, buffers, and preservatives. The conjugates can be administered also in liposomal compositions or as microemulsions. Systemic administration may also include relatively noninvasive methods such as the use of suppositories, transdermal patches, transmucosal delivery and intranasal administration. Oral administration is also suitable for conjugates herein. Suitable forms include syrups, capsules, and tablets, as is understood in the art.

The pharmaceutical compositions can be administered parenterally in the form of an injectable formulation. Pharmaceutical compositions for injection can be formulated using a sterile solution or any pharmaceutically acceptable liquid as a vehicle. Formulations may be prepared as solid forms suitable for solution or suspension in liquid prior to injection or as emulsions. Pharmaceutically acceptable vehicles include, but are not limited to, sterile water, physiological saline, and cell culture media (e.g., Dulbecco's Modified Eagle Medium (DMEM), α-Modified Eagles Medium (α-MEM), F-12 medium). Such injectable compositions may also contain amounts of nontoxic auxiliary substances such as wetting or emulsifying agents, pH buffering agents, such as sodium acetate and sorbitan monolaurate. Formulation methods are known in the art, see e.g., Pharmaceutical Preformulation and Formulation, 2nd Edition, M. Gibson, Taylor & Francis Group, CRC Press (2009).

The pharmaceutical compositions can be prepared in the form of an oral formulation. Formulations for oral use include tablets containing the active ingredient(s) in a mixture with non-toxic pharmaceutically acceptable excipients. These excipients may be, for example, inert diluents or fillers (e.g., sucrose, sorbitol, sugar, mannitol, microcrystalline cellulose, starches including potato starch, calcium carbonate, sodium chloride, lactose, calcium phosphate, calcium sulfate, or sodium phosphate); granulating and disintegrating agents (e.g., cellulose derivatives including microcrystalline cellulose, starches including potato starch, croscarmellose sodium, alginates, or alginic acid); binding agents (e.g., sucrose, glucose, sorbitol, acacia, alginic acid, sodium alginate, gelatin, starch, pregelatinized starch, microcrystalline cellulose, magnesium aluminum silicate, carboxymethylcellulose sodium, methylcellulose, hydroxypropyl methylcellulose, ethylcellulose, polyvinylpyrrolidone, or polyethylene glycol); and lubricating agents, glidants, and antiadhesives (e.g., magnesium stearate, zinc stearate, stearic acid, silicas, hydrogenated vegetable oils, or talc). Formulations for oral use may also be provided as chewable tablets, or as hard gelatin capsules wherein the active ingredient is mixed with an inert solid diluent (e.g., potato starch, lactose, microcrystalline cellulose, calcium carbonate, calcium phosphate or kaolin), or as soft gelatin capsules wherein the active ingredient is mixed with water or an oil medium, for example, peanut oil, liquid paraffin, or olive oil. Powders, granulates, and pellets may be prepared using the ingredients mentioned above under tablets and capsules in a conventional manner using, e.g., a mixer, a fluid bed apparatus or a spray drying equipment.

Other pharmaceutically acceptable excipients for oral formulations include, but are not limited to, colorants, flavoring agents, plasticizers, humectants, and buffering agents. Formulations for oral use may also be provided as chewable tablets, or as hard gelatin capsules wherein the active ingredient is mixed with an inert solid diluent (e.g., potato starch, lactose, microcrystalline cellulose, calcium carbonate, calcium phosphate or kaolin), or as soft gelatin capsules wherein the active ingredient is mixed with water or an oil medium, for example, peanut oil, liquid paraffin, or olive oil. Powders, granulates, and pellets may be prepared using the ingredients mentioned above under tablets and capsules in a conventional manner using, e.g., a mixer, a fluid bed apparatus or a spray drying equipment.

Dissolution or diffusion controlled release of a conjugate described herein (e.g., a conjugate of formula (1)) or a pharmaceutical composition thereof can be achieved by appropriate coating of a tablet, capsule, pellet, or granulate formulation of the conjugate, or by incorporating the conjugate into an appropriate matrix. A controlled release coating may include one or more of the coating substances mentioned above and/or, e.g., shellac, beeswax, glycowax, castor wax, carnauba wax, stearyl alcohol, glyceryl monostearate, glyceryl distearate, glycerol palmitostearate, ethylcellulose, acrylic resins, dl-polylactic acid, cellulose acetate butyrate, polyvinyl chloride, polyvinyl acetate, vinyl pyrrolidone, polyethylene, polymethacrylate, methylmethacrylate, 2-hydroxymethacrylate, methacrylate hydrogels, 1,3 butylene glycol, ethylene glycol methacrylate, and/or polyethylene glycols. In a controlled release matrix formulation, the matrix material may also include, e.g., hydrated methylcellulose, carnauba wax and stearyl alcohol, carbopol 934, silicone, glyceryl tristearate, methyl acrylate-methyl methacrylate, polyvinyl chloride, polyethylene, and/or halogenated fluorocarbon.

The pharmaceutical composition may be formed in a unit dose form as needed. The amount of active component, e.g., a conjugate described herein (e.g., a conjugate of formula (1)), included in the pharmaceutical compositions are such that a suitable dose within the designated range is provided (e.g., a dose within the range of 0.01-100 mg/kg of body weight).

### VII. Routes of Administration and Dosages

In any of the methods described herein, compositions described herein may be administered by any appropriate route for treating or protecting against an infection (e.g., a viral infection, a fungal infection, or a bacterial infection), or for preventing, stabilizing, or inhibiting the proliferation or spread of an infection (e.g., a viral infection, a fungal infection, or a bacterial infection). Compositions described herein may be administered to humans, domestic pets, livestock, or other animals with a pharmaceutically acceptable diluent, carrier, or excipient. In some embodiments, administering includes administration of any of the conjugates described herein (e.g., conjugates of formula (1)) or compositions intramuscularly, intravenously (e.g., as a sterile solution and in a solvent system suitable for intravenous use), intradermally, intraarterially, intraperitoneally, intralesionally, intracranially, intraarticularly, intraprostatically, intrapleurally, intratracheally, intranasally, intravitreally, intravaginally, intrarectally, topically, intratumorally, peritoneally, subcutaneously, subconjunctival, intravesicularlly, mucosally, intrapericardially, intraumbilically, intraocularally, orally (e.g., a tablet, capsule, caplet, gelcap, or syrup), topically (e.g., as a cream, gel, lotion, or ointment), locally, by inhalation, by injection, or by infusion (e.g., continuous infusion, localized perfusion bathing target cells directly, catheter, lavage, in cremes, or lipid compositions). In some embodiments, if a second therapeutic, such as an antiviral agent, is also administered in addition to a conjugate described herein, the antiviral agent or a pharmaceutical composition thereof may also be administered in any of the routes of administration described herein.

The dosage of a composition described herein (e.g., a conjugate of formula (1)) or pharmaceutical compositions thereof depends on factors including the route of administration, the disease to be treated (e.g., the extent and/or condition of the infection (e.g., viral infection, fungal infection, or bacterial infection)), and physical characteristics, e.g., age, weight, general health, of the subject. Typically, the amount of active contained within a single dose may be an amount that effectively prevents, delays, or treats the disorder without inducing significant toxicity. A pharmaceutical composition may include a dosage of a conjugate described herein ranging from 0.01 to 500 mg/kg (e.g., 0.01, 0.1, 0.2, 0.3, 0.4, 0.5, 1, 2, 3, 4, 5, 10, 15, 20, 25, 30, 35, 40, 45, 50, 100, 150, 200, 250, 300, 350, 400, 450, or 500 mg/kg) and, in a more specific embodiment, about 0.1 to about 30 mg/kg and, in a more specific embodiment, about 1 to about 30 mg/kg. In some embodiments, when a conjugate described herein (e.g., a conjugate of formula (1)) and an antiviral agent or antiviral vaccine are administered in combination (e.g., substantially simultaneously in the same or separate pharmaceutical compositions, or separately in the same treatment regimen), the dosage needed of the conjugate described herein may be lower than the dosage needed of the conjugate if the conjugate was used alone in a treatment regimen.

A composition described herein (e.g., a conjugate of formula (1)) or a pharmaceutical composition thereof may be administered to a subject in need thereof, for example, one or more times (e.g., 1-10 times or more; 1, 2, 3, 4, 5, 6, 7, 8, 9, or 10 times) daily, weekly, monthly, biannually, annually, or as medically necessary. Dosages may be provided in either a single or multiple dosage regimens. The timing between administrations may decrease as the medical condition improves or increase as the health of the patient declines. The dosage and frequency of administration may be adapted by the physician in accordance with conventional factors such as the extent of the infection and different parameters of the subject.

### VIII. Combination Therapies

It will also be appreciated that the conjugates, fusion proteins, and compositions of the present disclosure can be formulated and employed in combination therapies, that is, the conjugates, fusion proteins, and pharmaceutical compositions can be formulated with or administered concurrently with, prior to, or subsequent to, one or more other desired therapeutics or medical procedures. The particular combination of therapies (therapeutics or procedures) to employ in a combination regimen will take into account compatibility of the desired therapeutics and/or procedures and the desired therapeutic effect to be achieved. It will also be appreciated that the therapies employed may achieve a desired effect for the same disorder, or they may achieve different effects (e.g., control of any adverse effects). In preferred embodiments, the conjugate or fusion protein and the one or more other desired therapeutic agent are formulated in separate pharmaceutical compositions (e.g., formulated for different routes of administration). In some embodiments, the conjugate or fusion protein and the one or more other desired therapeutic agent are administered simultaneously (e.g., at substantially the same time, such as within 5 minutes, 30 minutes, 1-6 hours, 1-12 hours, or 1 day) or sequentially (e.g., at different times, such as more than 1 day apart). Provided the one or more other desired therapeutic agents and the conjugate or fusion protein are administered sequentially, the one or more other desired therapeutic agents are administered 1-50 (e.g., 1-15, 10-25, 20-35, 30-45, or 35-50) times after the administration of the conjugate or fusion protein (e.g., administrations 1 day, 2, days, 5, days, 1 week, 2 weeks, 3 weeks, 1 month, 2 months, 6 months, or 12 months, or more after the conjugate or fusion protein).

### Antiviral Agents

In some embodiments, one or more antiviral agents may be administered in combination with a conjugate described herein (e.g., a conjugate of any one of formula (1)) or a fusion protein described herein.

In some embodiments the antiviral is selected from the group consisting of vidarabine, acyclovir, gancyclovir, valgancyclovir, a nucleoside-analog reverse transcriptase inhibitor (e.g., AZT (Zidovudine), ddl (Didanosine), ddC (Zalcitabine), d4T (Stavudine), or 3TC (Lamivudine)), a non-nucleoside reverse transcriptase inhibitor (e.g., (nevirapine or delavirdine), protease inhibitor (saquinavir, ritonavir, indinavir, or nelfinavir), ribavirin, or interferon). The preceding list is meant to be exemplary of antivirals known to one skilled in the art for the treatment of infection and is not meant to limit the scope of the invention.

### Antiviral Vaccines

In some embodiments, any one of conjugates described herein (e.g., a conjugate of formula (1)) is administered in combination with an antiviral vaccine (e.g., a composition that elicits an immune response in a subject directed against a virus).

In some embodiments the viral vaccine includes an immunogen that elicits an immune response in the subject against influenza virus A, B, C, or parainfluenza virus. In some embodiments the immunogen is an inactivated virus (e.g., the vaccine is a trivalent influenza vaccine that contains purified and inactivated material influenza virus A, B, C, or parainfluenza virus or any combination thereof). In some embodiments the vaccine is given as an intramuscular injection. In some embodiments, the vaccine is a live virus vaccine that contains live viruses that have been attenuated (weakened). In some embodiments the vaccine is administered as a nasal spray.

### Antibacterial Agents

In some embodiments, one or more antibacterial agents may be administered in combination with a conjugate described herein (e.g., a conjugate of any one of formula (1)) or a fusion protein described herein.

The antibacterial agent may be selected from the group consisting of amikacin, gentamicin, kanamycin, neomycin, netilmicin, tobramycin, paromomycin, streptomycin, spectinomycin, geldanamycin, herbimycin, rifaximin, loracarbef, ertapenem, doripenem, imipenem/cilastatin, meropenem, cefadroxil, cefazolin, cefalotin, cefalexin, cefaclor, cefamandole, cefoxitin, cefprozil, cefuroxime, cefixime, cefdinir, cefditoren, cefoperazone, cefotaxime, cefpodoxime, ceftazidime, ceftibuten, ceftizoxime, ceftriaxone, cefepime, ceftaroline fosamil, ceftobiprole, teicoplanin, vancomycin, telavancin, dalbavancin, oritavancin, clindamycin, lincomycin, daptomycin, azithromycin, clarithromycin, dirithromycin, erythromycin, roxithromycin, troleandomycin, telithromycin, spiramycin, aztreonam, furazolidone, nitrofurantoin, linezolid, posizolid, radezolid, torezolid, amoxicillin, ampicillin, azlocillin, carbenicillin, cloxacillin, dicloxacillin, flucloxacillin, mezlocillin, methicillin, nafcillin, oxacillin, penicillin g, penicillin v, piperacillin, penicillin g, temocillin, ticarcillin, amoxicillin clavulanate, ampicillin/sulbactam, piperacillin/tazobactam, ticarcillin/clavulanate, bacitracin, colistin, polymyxin b, ciprofloxacin, enoxacin, gatifloxacin, gemifloxacin, levofloxacin, lomefloxacin, moxifloxacin, nalidixic acid, norfloxacin, ofloxacin, trovafloxacin, grepafloxacin, sparfloxacin, temafloxacin, mafenide, sulfacetamide, sulfadiazine, silver sulfadiazine, sulfadimethoxine, sulfamethizole, sulfamethoxazole, sulfanilimide, sulfasalazine, sulfisoxazole, trimethoprim-sulfamethoxazole (tmp-smx), sulfonamidochrysoidine, demeclocycline, doxycycline, minocycline, oxytetracycline, tetracycline, clofazimine, dapsone, capreomycin, cycloserine, ethambutol(bs), ethionamide, isoniazid, pyrazinamide, rifampicin, rifabutin, rifapentine, streptomycin, arsphenamine, chloramphenicol, fosfomycin, fusidic acid, metronidazole, mupirocin, platensimycin, quinupristin/dalfopristin, thiamphenicol, tigecycline, tinidazole, and trimethoprim. The preceding list is meant to be exemplary of antibacterials known to one skilled in the art for the treatment of infection and is not meant to limit the scope of the invention.

### Antifungal Agents

In some embodiments, one or more antifungal agents may be administered in combination with a conjugate described herein (e.g., a conjugate of any one of formula (1)) or a fusion protein described herein.

In some embodiments of the above-described combination therapies for the treatment of infection in a subject in need thereof, the antifungal is selected from the group consisting of rezafungin, amphotericin B, candicidin, filipin, hamycin, natamycin, nystatin, rimocidin, bifonazole, butoconazole, clotrimazole, econazole, fenticonazole, isoconazole, ketoconazole, luliconazole, miconazole, omoconazole, oxiconazole, sertaconazole, sulconazole, tioconazole, triazoles, albaconazole, efinaconazole, epoxiconazole, fluconazole, isavuconazole, itraconazole, posaconazole, propiconazole, ravuconazole, terconazole, voriconazole, thiazoles, abafungin, amorolfin, butenafine, naftifine, terbinafine, anidulafungin, caspofungin, micafungin, ciclopirox, flucytosine, griseofulvin, tolnaftate, and undecylenic acid. The preceding list is meant to be exemplary of antifungals known to one skilled in the art for the treatment of infection and is not meant to limit the scope of the invention.

### EXAMPLES

The following examples are put forth so as to provide those of ordinary skill in the art with a description of how the compositions and methods described herein may be used, made, and evaluated, and are intended to be purely exemplary of the invention and are not intended to limit the scope of what the inventors regard as their invention.

### Example 1. General Procedure for Synthesis of Azido Fc

Preparation of PEG4-azido NHS ester solution (0.050 M) in DMF/PBS: 16.75 mg of PEG4-azido NHS ester was dissolved in 0.100 mL of DMF at 0° C. and diluted to 0.837 mL by adding PBS 1× buffer at 0° C. This solution was used for preparing other PEG4-azido Fc with a variety of DAR values by adjusting the equivalents of this PEG4-azido NHS ester PBS solution.

Pretreatment of h-IgG1 Fc (107.2 mg in 8.800 mL of pH 7.4 PBS, MW-57891 Da, 1.852 μmol): The Fc solution was transferred into four centrifugal concentrators (30,000 MWCO, 15 mL) and diluted to 15 mL with PBS×1 buffer and concentrated to a volume of ˜1.5 mL. The residue was diluted 1:10 in PBS pH 7.4, and concentrated again. This wash procedure was repeated for total of four times followed by dilution to 8.80 mL.

Preparation of PEG4-azido Fc: 0.050M PEG4-azidoNHS ester PBS buffer solution (0.593 mL, 29.6 μmol, 16 equivalents) was added to above solution of h-IgG1 Fc (SEQ ID NO: 21; the C-terminal Lys is proteolytically cleaved after expression and the mixture was shaken rotated for 2 hours at ambient temperature. The solution was concentrated by using four centrifugal concentrators (30,000 MWCO, 15 mL) to a volume of ˜1.5 mL. The crude mixture was diluted 1:10 in PBS pH 7.4, and concentrated again. This wash procedure was repeated for total of three times. The concentrated Fc-PEG4-azide was diluted to 8.80 mL with pH 7.4 PBS buffer and ready for Click conjugation. The purified material was quantified using a NANODROP™ UV visible spectrophotometer (using a calculated extinction coefficient based on the amino acid sequence of h-IgG1). Yield was quantitative after purification.

### Example 2. Synthesis of Conjugate 1 (Fc Domain Including C220S/M252Y/S254T/T256E Mutations)

Preparation of the Click reagent solution: 0.0050M CuSO₄ in PBS buffer solution: 10.0 mg CuSO₄ was dissolved in 12.53 mL PBS, then took 5.00 mL this CuSO₄ solution and added 43.1 mg BTTAA (CAS #1334179-85-9) and 247.5 mg sodium ascorbate to give the Click reagent solution (0.0050M CuSO4, 0.020M BTTAA and 0.25M sodium ascorbate).

To a solution of azido functionalized Fc (Example 1; 65.5 mg, 10.0 mL, 1.13 μmol, azido DAR-5.9, SEQ ID NO: 10) in a 15 mL centrifuge tube was added to alkyne derivatized small molecule viral inhibitor (22.7 mg, 15.2 μmol, 3.0 equivalents per each azido of the Fc). After gently agitating to dissolve all solids, the mixture was treated with the Click reagent solution (1.80 mL). The resulting mixture was gently rotated for 12 hours at ambient temperature. It was purified by affinity chromatography over a protein A column, followed size exclusion chromatography. Maldi TOF analysis of the purified final product gave an average mass of 66,420 Da (DAR=5.8). Yield 57 mg with 98% purity.

### Example 3. Synthesis of Conjugate 2 (Fc Domain Including C220S Mutation)

This conjugate was prepared analogously to conjugate 1 (Example 2) by PEG4-azido-Fc (SEQ ID NO: 21, prepared as in Example 1) and a small molecule viral inhibitor. Maldi TOF analysis of the purified final product gave an average mass of 62,927 Da (DAR=4.2).

### Example 4. 30-Day Comparative Non-Human Primate PK Study Following IV Administration of Conjugate 1 and Conjugate 2

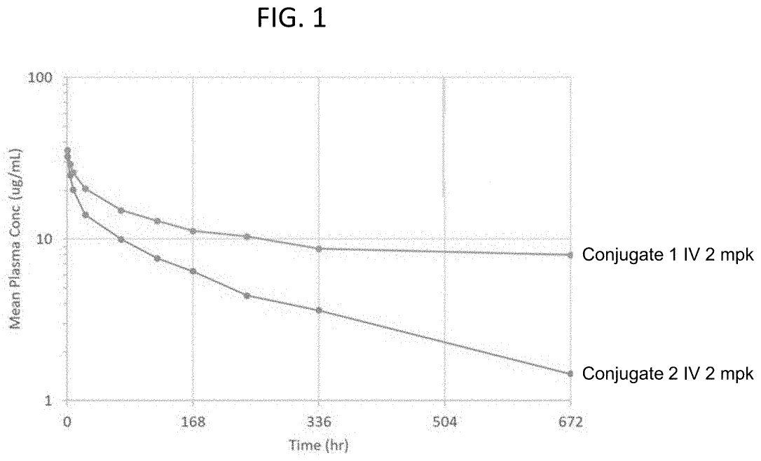

Non-human primate (NHP) PK studies were performed by BTS Research (San Diego, Calif.) using male and female cynomolgus monkeys 5-9 years old with body weights ranging from 3.5-8.5 kg. NHPs were injected IV with 2 mg/kg of test article (0.4 mL/kg dose volume). Animals were housed under standard IACUC approved housing conditions. At appropriate times animals were non-terminally bled (via femoral or cephalic veins) with blood collected in K₂EDTA tubes to prevent coagulation. Collected blood was centrifuged (2,000×g, for 10 minutes) and plasma withdrawn for analysis of test article concentrations over time. The plasma concentrations for Conjugate 1 (C220S/M252Y/S254T/T256E) and Conjugate 2 (C220S) at each time point were measured by sandwich ELISA. Briefly, test articles were captured on Fc-coated plates and then detected using a HRP-conjugated anti-human IgG-Fc antibody. Protein concentrations were calculated in GraphPad Prism using 4PL non-linear regression of Conjugate 1 or Conjugate 2 standard curves. The curves comparing Conjugate 1 and Conjugate 2 are shown in FIG. 1. Conjugate 1 demonstrates a significantly improved terminal half-life of ˜45 days compared with ˜10 days for Conjugate 2. AUCs for Conjugate 1 are 2× greater than the AUCs for Conjugate 2 (Table 2).

TABLE 1
Monkey PK, Conjugate 1 vs. Conjugate 2

| | | | | Time (hr) | | | | | | | | | | | | | |
|---|---|---|---|---|---|---|---|---|---|---|---|---|---|---|---|---|---|
| | | | | 0.25 | 4 | 8 | 24 | 72 | 120 | 168 | 240 | 336 | 672 | | | | |
| Dose | | | | Conc | | | | | | | | | | Tmax | Cmax | AUClast | Half-life |
| (mg/kg) | Route | Conjugate | | (ug/mL) | | | | | | | | | | (hr) | (ug/mL) | (hr*ug/mL) | (hr) |
| 2 | IV | Conjugate 1 | Mean | 35.4 | 29 | 25.7 | 20.5 | 15.1 | 13 | 11.2 | 10.4 | 8.71 | 7.97 | 0.25 | 35.4 | 7210 | 1080 |
| 2 | IV | Conjugate 2 | Mean | 32.6 | 24.8 | 20.1 | 14.1 | 9.97 | 7.61 | 6.33 | 4.47 | 3.62 | 1.47 | 0.25 | 32.6 | 3450 | 249 |

### Example 5. 14-Day Mouse PK Study of Plasma and Epithelial Lining Fluid (ELF) Concentrations of Conjugate 2

Female BALB/c mice from Charles River Laboratories were allowed to acclimate for 5 days prior to study commencement. Animals were housed 3-6 per cage with free access to food and water. All procedures were performed to NeoSome IACUC policies and guidelines. Mice were injected subcutaneously (SC) with 20 mg/kg of test article (10 mL/kg dose volume). At selected time points, 3 mice were euthanized by CO₂ inhalation. Blood was collected through cardiac puncture into K₂EDTA tubes for plasma retention. Following blood collection, a bronchoalveolar lavage (BAL) was performed by exposing the trachea, inserting a 23G tubing adaptor, and performing 2×0.5 mL flushes with sterile 1× PBS pH 7.4. The recovered fluid volume was recorded and retained. Once the BAL procedure was complete, the lungs were removed, weighed and stored at −80° C. Aliquots of the plasma and BAL fluid (BALF) were decanted prior to −80° C. storage of the samples for use in a urea quantification assay. The collected BALF was centrifuged at 12,000 RPM for 5 minutes at room temperature to pellet the alveolar macrophages with both the pellet and supernatant stored at −80° C. until shipment to sponsor. The plasma concentrations for conjugate 2 at each time point were measured by indirect ELISA as described in detail above. Briefly, conjugate 2 molecules were captured on small molecule viral target coated plates and then detected using a HRP-conjugated anti-human IgG Fcγ specific F(ab′)₂. The same ELISA was performed on BALF harvested as described above. Conjugate 2 plasma concentrations were calculated in GraphPad Prism using 4PL non-linear regression of conjugate 2 standard curves. ELF volume and conjugate 2 concentration in ELF was determined using urea as a dilution marker as described previously (Rennard et al., 1986 J Appl Physiol 60:532-538). The curves comparing conjugate 2 to ELF levels are shown in FIG. 2. By 2 h post injection, conjugate 2 epithelial lining fluid (ELF) levels are ˜60% of plasma exposure levels (AUCs) across the rest of the time course indicating nearly immediate partitioning of conjugate 2 from plasma to the ELF in the lung (FIG. 2, Table 2).

TABLE 2
Conjugate 2 plasma and ELF levels in mice over 2 weeks.

| | Time | | | | | | | | | | | | |
|---|---|---|---|---|---|---|---|---|---|---|---|---|---|
| | (hr) | | | | | | | | | | | | |
| | 1 | 2 | 4 | 8 | 24 | 48 | 72 | 120 | 168 | 336 | | | |
| | Conc | | | | | | | | | | Tmax | Cmax | AUClast |
| Group | (ug/mL) | | | | | | | | | | (hr) | (ug/mL) | (hr*ug/mL) |
| ELF | 5.61 | 29.9 | 70.6 | 98.4 | 149 | 105 | 94.2 | 49.5 | 47.4 | 16.1 | 24 | 149 | 19000 |
| Plasma | 30.7 | 63.9 | 110 | 180 | 197 | 178 | 144 | 104 | 87 | 29.4 | 24 | 197 | 32500 |

### Example 6. 7-Day Mouse PK Study Comparing SC Administration of Conjugate 1 and Conjugate 2

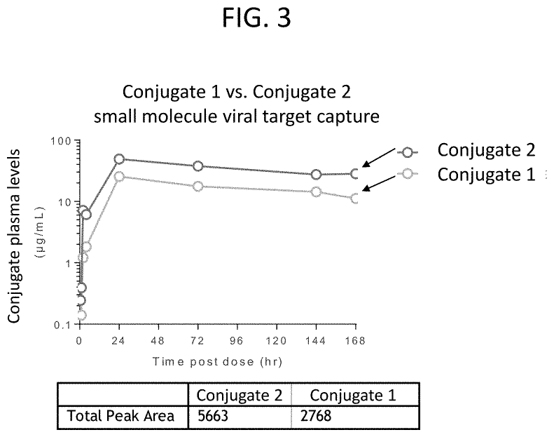

Mouse PK studies were performed using male CD-1 mice 6 weeks of age. Mice were injected SC with 10 mg/kg of test article (10 mL/kg dose volume). Animals were housed under standard IACUC approved housing conditions. At appropriate times animals were non-terminally bled (retro-orbital, cheek, or by tail vein) with blood collected in K₂EDTA tubes to prevent coagulation. Collected blood was centrifuged (2,000×g, for 10 minutes) and plasma withdrawn for analysis of test article concentrations over time. The plasma concentrations for conjugate 2 at each time point were measured by indirect ELISA as described in detail above. Briefly, conjugate 2 molecules were captured on small molecule viral target coated plates and then detected using a HRP-conjugated anti-human IgG Fcγ specific F(ab′)₂. Protein concentration was calculated in GraphPad Prism using 4PL non-linear regression of conjugate 2 standard curves. The curves comparing the 7-day PK profiles of conjugate 2 and conjugate 1 are shown in FIG. 3. The plasma exposure levels for conjugate 2 (C220S) were approximately 50% greater than for conjugate 1 (C220S/M252Y/S254T/T256E). Compared to WT human IgG1, the half-life of human IgG1 YTE Fc variant is known to be reduced in mice due to enhanced mouse FcRn binding at neutral pH, which negates the improved binding to mouse FcRn at acidic pH (Dall'Acqua et al. 2002 J Immunol 169:5171-5180).

### Example 7. 7-Day Mouse PK Study Comparing IV Administration of Fcs with Different Molecular Weights

Mouse PK studies were performed using male CD-1 mice 6 weeks of age. Mice were injected intravenously (IV) via the tail vein with 5 mg/kg of test article (5 mL/kg dose volume). Animals were housed under standard IACUC approved housing conditions. At appropriate times animals were non-terminally bled (retro-orbital, cheek, or by tail vein) with blood collected in K₂EDTA tubes to prevent coagulation. Collected blood was centrifuged (2,000×g, for 10 min) and plasma withdrawn for analysis of test article concentrations over time. The Fc plasma concentrations at each time point were measured by Fc-capture ELISA as follows. Nunc Maxisorp 96-well plates (cat no. 12-565-136, Fisher Scientific) were coated overnight at 4° C. with 0.1 μg/100 μL/well of goat anti-human IgG (Fcγ fragment specific; cat no. 109-005-098, Jackson Immunoresearch) in carbonate buffer (cat no. C3041, MilliporeSigma). Plates were washed 5× with 300 μL/well PBST and blocked with 200 μL/well 5% non-fat dry milk (cat no. 9999S, Cell Signaling) in PBST for 1 h at room temperature with shaking. Three-fold serial dilutions of the plasma samples were plated at 100 μL/well and incubated at room temperature for 2 h with shaking (sample diluent: 2.5% non-fat dry milk in PBS 0.025% Tween 20+naïve mouse plasma final concentration of 1:900). Fc standard curves ranging from 0.03 to 55 ng/mL in duplicate, were run on each plate. Following the 2 h incubation, plates were washed 5× with 300 μL/well PBST. Test articles (Fcs) bound to capture antibodies on the plates were then probed with 100 μL/well of HRP conjugated anti-human IgG Fc F(ab′)2 (cat no. 709-036-098, Jackson Immunoresearch) diluted 1:2,000 in sample diluent for 1 h at room temp with shaking. Plates were then washed 8× in 300 μL/well PBST and developed with 100 μL/well TMB substrate reagent (cat no. 555214, BD) for 7-8 minutes. The reaction was stopped with 100 μL/well 1 N H₂SO₄ and the absorbance read at 450 nm with an EnSpire multimode plate reader (PerkinElmer). Test articles in plasma samples were interpolated using GraphPad Prism Version 8 following nonlinear regression analysis (Sigmoidal, 4PL analysis) of the standard curves. The resulting mean plasma concentrations were then used to calculate the total AUC for each plasma concentration-time profile.

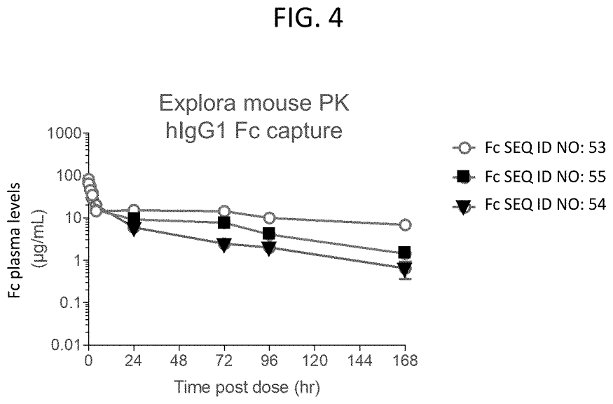

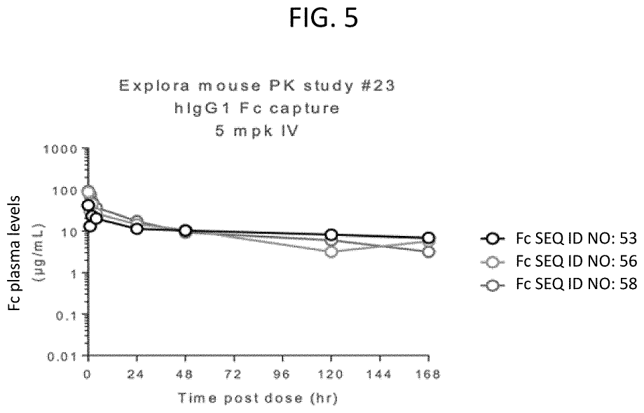

Mouse PK studies were performed to optimize the PK (by reducing clearance) based on Fc domain monomer length and molecular weight (Table 3 and 4 and FIGS. 4 and 5). Slower clearance was observed for a longer Fc domain containing an extended N-terminus comprising non-germline amino acids and a C-terminal affinity tag (Fc domain homodimer of SEQ ID NO: 53, MW: 58,272 Da). The removal of the potentially immunogenic N-terminal and C-terminal extensions from the Fc domain monomer (Fc domain homodimer of SEQ ID NO: 54, MW: 53,743 Da) resulted in a smaller Fc domain that was cleared more rapidly from mouse plasma, possibly via renal filtration. To improve the PK parameters to more closely resemble those seen in the larger Fc domain with non-endogenous N-terminal and C-terminal extensions, 6 amino acid residues from the endogenous IgG1 sequence were included on the N-terminal end of an Fc domain monomer (Fc domain homodimer of SEQ ID NO: 55, MW: 55,031 Da) showing improved Fc domain PK parameters (i.e. reduced clearance), but the Fc N-terminal elongation was unable to restore PK values to that seen in the Fc domain including undesirable N-terminal and C-terminal tags (Fc domain homodimer of SEQ ID NO: 53) (Data shown in Table 3 and FIG. 4).

TABLE 3
7-day Mouse PK #19

| | | Plasma Concentration (μg/ml) | | |
|---|---|---|---|---|
| | | Fc SEQ ID | Fc SEQ ID | Fc SEQ ID |
| Time | Mouse | NO: 53 | NO: 55 | NO: 54 |
| (hr) | No. | 5 mpk IV | 5 mpk IV | 5 mpk IV |
| 0.083 | 1 | 64.27888626 | 81.12820802 | 69.54669113 |
| 1 | 2 | 44.90558715 | 55.58212615 | 48.04730329 |
| 2 | 1 | 34.6927259. | 39.79925941 | 28.64695224 |
| 4 | 2 | 14.39948664 | 15.17752287 | 20.01025194 |
| 24 | 1 | 15.24637179 | 9.238470015 | 6.000155484 |
| 72 | 2 | 14.34487176 | 7.64802396 | 2.450216864 |
| 96 | 1 | 9.905107721 | 4.027415596 | 2.008274435 |
| 168 | 2 | 6.862316753 | 1.436095949 | 0.645661916 |

| | | | |
|---|---|---|---|
| AUC | 2,040 | 1,152 | 753 |

ISAMVRSDKTHTCPPCPAPELLGGPSVFLFPPKPKDTLMISRTPEVTCVV
VDVSHEDPEVKFNWYVDGVEVHNAKTKPREEQYNSTYRVVSVLTVLHQDW
LNGKEYKCKVSNKALPAPIEKTISKAKGQPREPQVYTLPPSR [symbol] E [symbol] TKN
QVSLTCLVKGFYPSDIAVEWESNGQPENNYKTTPPVLDSDGSFFLYSKLT
VDKSRWQQGNVFSCSVMHEALHNHYTQKSLSLSPGGGGGSEQKLISEEDL

SEQ ID NO: 54: mature human Fc IgGl, N-termina ISAMVRS amino acid residues added (italicized), allotype G1m(fa) (bold italics)

KTHTCPPCPAPELLGGPSVFLFPPKPKDTLMISRTPEVTCVVVDVSHEDP
EVKFNWYVDGVEVHNAKTKPREEQYNSTYRVVSVLTVLHQDWLNGKEYKC
KVSNKALPAPIEKTISKAKGQPREPQVYTLPPSR [symbol] E [symbol] TKNQVSLTCLVKG
FYPSDIAVEWESNGQPENNYKTTPPVLDSDGSFFLYSKLTVDKSRWQQGN
VFSCSVMHEALHNHYTQKSLSLSPG

SEQ ID NO: 55: mature human Fc IgGl. N-termina amino acid residues added (italicized), hinge residues are italicized allotype G1m(fa) (bold italics)

EPKSS(#)DKTHTCPPCPAPELLGGPSVFLFPPKPKDTLMISRTPEVTCV
VVDVSHEDPEVKFNWYVDGVEVHNAKTKPREEQYNSTYRVVSVLTVLHQD
WLNGKEYKCKVSNKALPAPIEKTISKAKGQPREPQVYTLPPSR [symbol] E [symbol] TKNQ
VSLTCLVKGFYPSDIAVEWESNGQPENNYKTTPPVLDSDGSFFLYSKLTV
DKSRWQQGNVFSCSVMHEALHNHYTQKSLSLSPG

To develop a new Fc domain that more closely resembled an endogenous IgG1 domain with PK parameters similar to the Fc of SEQ ID NO: 53, further mouse PK studies were performed with Fc domain monomers including endogenous amino acids on the N-terminal end extending into the Fab region of an antibody (Fc domain homodimer of SEQ ID NO: 56, MW: 58,154 Da). The study showed that Fc domain monomers including amino acid residues extending into the Fab region of an antibody demonstrated surprising improvements in PK parameters (data shown in Table 4 and FIG. 5). Overall, improvements in plasma exposure levels were observed with increasing molecular weight. Specifically, AUCs for an Fc domain having a molecular weight of 55,031 Da ((homodimer of SEQ ID NO: 55 were greater than an Fc domain having a molecular weight of 53,743 Da (homodimer of SEQ ID NO: 54). Further, adding N-terminal Fab residues producing an Fc domain having a molecular weight of 58,154 Da (SEQ ID NO: 56) further improved AUC for the Fc domain. Including additional Fab residues on the N-terminal end of the Fc domain monomers is believed to introduce undesirable characteristics, including, unpaired cysteines, hydrophobic regions and secondary structure of the endogenous Fab region, which might negatively impact solution properties and promote aggregation while not significantly reducing clearance from plasma.

TABLE 4
7-day Mouse PK#22

| | Average Plasma Concentration (μg/ml) | | |
|---|---|---|---|
| Time (hr) | Fc SEQ ID NO: 53 | Fc SEQ ID NO: 56 | Fc SEQ ID NO: 58 |
| 0.083 | 42.18 | 87.63 | 92.92 |
| 1 | 13.12 | 30.47 | 75.61 |
| 2 | 23.25 | 45.78 | 51.08 |
| 4 | 20.31 | 26.56 | 37.11 |
| 24 | 11.44 | 14.70 | 17.56 |
| 72 | 10.38 | 10.78 | 9.27 |
| 96 | 8.23 | 3.17 | 6.08 |
| 168 | 6.90 | 5.78 | 3.17 |
| AUC | 1,699 | 1,600 | 1,872 |

NVNHKPSNTKVDKKVEPKSS(#)DKTHTCPPCPAPELLGGPSVFLFPPKP
KDTLMISRTPEVTCVVVDVSHEDPEVKFNWYVDGVEVHNAKTKPREEQYN
STYRVVSVLTVLHQDWLNGKEYKCKVSNKALPAPIEKTISKAKGQPREPQ
VYTLPPSR [symbol] E [symbol] TKNQVSLTCLVKGFYPSDIAVEWESNGQPENNYKTTP
PVLDSDGSFFLYSKLTVDKSRWQQGNVFSCSVMHEALHNHYTQKSLSLSPG

SEQ ID NO: 58: mature human IgG1 Fc, Cys to Ser substitution (#), M428L, N434S (Bold/Underlined), allotype G1m(fa) (bold italics), N-terminal Fab residues are underlined, hinge residues are italicized

NVNHKPSNTKVDKKVEPKSS(#)DKTHTCPPCPAPELLGGPSVFLFPPKP
KDTLMISRTPEVTCVVVDVSHEDPEVKFNWYVDGVEVHNAKTKPREEQYN
STYRVVSVLTVLHQDWLNGKEYKCKVSNKALPAPIEKTISKAKGQPREPQ
VYTLPPSR [symbol] E [symbol] TKNQVSLTCLVKGFYPSDIAVEWESNGQPENNYKTTPPV
LDSDGSFFLYSKLTVDKSRWQQGNVFSCSVLHEALHSHYTQKSLSLSPG

1. A variant Fc domain monomer, wherein the variant Fc domain monomer comprises substitutions at position 220 and positions 252, 254, and 256 or positions 309, 311, and 434, wherein numbering is according to the EU index as in Kabat, and wherein the substitution at position 220 is a serine, the substitution at position 252 is a tyrosine, the substitution at position at position 254 is a threonine, the substitution at position 256 is a glutamic acid, the substitution at position 309 is an aspartic acid, the substitution at position at position 311 is a histidine, and the substitution at position 434 is a serine.

2. The variant Fc domain monomer of claim 1, wherein the variant Fc domain monomer comprises substitutions at positions 220, 252, 254, and 256, wherein numbering is according to the EU index as in Kabat, and wherein the substitution at position 220 is a serine, the substitution at position 252 is a tyrosine, the substitution at position at position 254 is a threonine, and the substitution at position 256 is a glutamic acid.

3. The variant Fc domain monomer of claim 2, wherein the variant Fc domain monomer is a variant of human IgG1 or human IgG2.

4. The variant Fc domain monomer of claim 2 or 3, wherein the substitution at position 220 a cysteine to serine (C220S).

5. The variant Fc domain monomer of any one of claims 2-4, wherein the substitution at position 252 is a methionine to tyrosine (M252Y).

6. The variant Fc domain monomer of any one of claims 2-5, wherein the substitution at position 254 is a serine to threonine (S254T).

7. The variant Fc domain monomer of any one of claims 2-6, wherein the substitution at position 256 is a threonine to glutamate (T256E).

8. The variant Fc domain monomer of claim 1, wherein the variant Fc domain monomer comprises substitutions at positions 220, 309, 311, and 434, wherein numbering is according to the EU index as in Kabat, and wherein the substitution at position 220 is a serine, the substitution at position 309 is an aspartic acid, the substitution at position at position 311 is a histidine, and the substitution at position 434 is a serine.

9. The variant Fc domain monomer of claim 8, wherein the variant Fc domain monomer is a variant of human IgG1 or human IgG2.

10. The variant Fc domain monomer of claim 8 or 9, wherein the substitution at position 220 a cysteine to serine (C220S).

11. The variant Fc domain monomer of any one of claims 8-10, wherein the substitution at position 309 is a valine to aspartic acid (V309D).

12. The variant Fc domain monomer of any one of claims 8-11, wherein the substitution at position 311 is a glutamine to histidine (Q311H).

13. The variant Fc domain monomer of any one of claims 8-12, wherein the substitution at position 434 is an asparagine to serine (N434S).

14. The variant Fc domain monomer of any one of claims 1-13, wherein the variant Fc domain monomer comprises less than 300 amino acid residues.

15. The variant Fc domain monomer of any one of claims 1-14, wherein the variant Fc domain monomer comprises at least 200 amino acid residues.

16. The variant Fc domain monomer of any one of claims 1-15, wherein the variant Fc domain monomer comprises an amino acid sequence at least 90% identical to the sequence of SEQ ID NO: 1-52, or a region thereof.

17. A variant Fc domain monomer comprising a serine at amino acid position 220, wherein the amino acid numbering is according to the EU index as in Kabat, and wherein the variant Fc domain monomer is between 200 and 300 amino acid residues in length.

18. The variant Fc domain monomer of claim 17, wherein the variant Fc domain monomer is between 240 and 255 amino acid residues in length.

19. A variant Fc domain monomer comprising a serine at amino acid position 220, wherein the amino acid numbering is according to the EU index as in Kabat, and wherein the variant Fc domain monomer is between about 20 kDa and about 40 kDa in mass.

20. The variant Fc domain monomer of claim 19, wherein the variant Fc domain monomer is between about 25 kDa and 28 kDa in mass.

21. The variant Fc domain monomer of any one of claims 17-19, wherein the variant Fc domain monomer is a variant of human IgG1 or human IgG2.

22. The variant Fc domain monomer of claim 21, wherein the variant Fc domain monomer is a variant of human IgG1.

23. The variant Fc domain monomer of any one of claims 17-22, wherein the N-terminus of the variant Fc domain monomer comprises between 10 and 20 residues of the Fab domain.

24. The variant Fc domain monomer of claim 23, wherein the N-terminus of the variant Fc domain monomer comprises an N-terminus of any one of amino acid residues 198-205.

25. The variant Fc domain monomer of claim 24, wherein the variant Fc domain monomer comprises an N-terminus of amino acid residue Asn 201.

26. The variant Fc domain monomer of claim 24, wherein the variant Fc domain monomer comprises an N-terminus of amino acid residue Val 202.

27. The variant Fc domain monomer of any one of claims 17-26, wherein the variant Fc domain monomer comprises a C-terminus of any one of amino acid residues 437-447.

28. The variant Fc domain monomer of claim 27, wherein the variant Fc domain monomer comprises a C-terminus of amino acid residue Gly 446.

29. The variant Fc domain monomer of claim 27, wherein the variant Fc domain monomer comprises a C-terminus of amino acid residue Lys 447.

30. The variant Fc domain monomer of any one of claims 17-29, wherein the variant Fc domain monomer further comprises substitutions at positions 252, 254, and 256, wherein the substitution at position 252 is a methionine to tyrosine (M252Y), the substitution at position 254 is a serine to threonine (S254T), and the substitution at position 256 is a threonine to glutamate (T256E).

31. The variant Fc domain monomer of any one of claims 17-29, wherein the variant Fc domain monomer further comprises substitutions at positions 309, 311, and 434, wherein the substitution at position 309 is a valine to aspartic acid (V309D), the substitution at position 311 is a glutamine to histidine (Q311H), and the substitution at position 434 is an asparagine to serine (N434S).

32. The variant Fc domain monomer of claim any one of claims 17-31, comprising an amino acid sequence at least 90% identical to the sequence of SEQ ID NOs: 20-52 or 56-58 or a region thereof.

33. A variant Fc domain comprising a dimer of variant Fc domain monomers each independently selected from a variant Fc domain monomer of any one of claims 1-32, wherein the variant Fc domain is between about 50 kDa and about 70 kDa in mass.

34. A conjugate comprising a variant Fc domain monomer and at least one therapeutic agent, wherein the variant Fc domain monomer is covalently conjugated to the therapeutic agent by a linker.

35. The conjugate of claim 34, wherein the conjugate is described by formula (1): 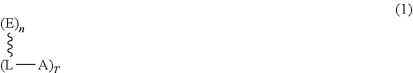
wherein each A is independently a therapeutic agent;
each E comprises a variant Fc domain monomer of any one of claims 1-21;
L is a linker;
n is 1 or 2;
T is an integer from 1 to 20; and
the squiggly line connected to the E indicates that each L-A is covalently attached to E,
or a pharmaceutcably acceptable salt thereof.

36. The conjugate of claim 35, wherein the therapeutic agent is an antiviral agent, an antifungal agent, or an antibacterial agent.

37. The conjugate of claim 36, wherein the therapeutic agent is an antiviral agent.

38. The conjugate of claim 37, wherein the therapeutic agent is an antifungal agent.

39. The conjugate of claim 37, wherein the therapeutic agent is an antibacterial agent.

40. A fusion protein comprising a variant Fc domain monomer and at least one polypeptide therapeutic agent, wherein the variant Fc domain monomer is covalently conjugated to the polypeptide therapeutic agent by a linker.

41. The fusion protein of claim 40, wherein the fusion protein comprises the structure:
(P₂-L₂)ₙ₂-B-(L₁-P₁)ₙ₁
wherein B is a variant Fc domain monomer of any one of claims 1-33 or a conjugate of any one of claims 34-39;
P₁ and P₂ are each independently a polypeptide therapeutic agent;
L₁ and L₂ are each independently a linker; and
n₁ and n₂ are each independently 0 or 1, wherein at least one of n₁ and n₂ is 1.

42. The fusion protein of claim 41, wherein n₁ is 1, n₂ is 0, and the fusion protein comprises the structure:
B-L₁-P₁.

43. The fusion protein of claim 42, wherein the linker (L₁) is conjugated to C-terminus of the Fc domain monomer (B) and to the N-terminus of the polypeptide therapeutic agent (P₁).

44. The fusion protein of claim 43, wherein the linker (L₁) is conjugated to N-terminus of the Fc domain monomer (B) and to the C-terminus of the polypeptide therapeutic agent (P₁).

45. The fusion protein of any one of claims 42-44, wherein L₁ is a peptide linker comprising between 2 and 200 amino acids.

46. The fusion protein of claim 45, wherein L₁ is a peptide linker comprising between 5 and 25 amino acids.

47. The fusion protein of any one of claims 42-46, wherein L₁ is a peptide linker comprising the amino acid sequence of any one of (GS)ₓ, (GGS)ₓ, (GGGGS)ₓ, (GGSG)ₓ, (SGGG)ₓ, wherein x is an integer from 1 to 10.

48. The fusion protein of any one of claims 42-47, wherein B, L₁, and P₁ are expressed as a single polypeptide chain.

49. The fusion protein of claim 42, wherein the linker (L₁) is conjugated to N-terminus of the Fc domain monomer (B) and to the N-terminus of the polypeptide therapeutic agent (P₁).

50. The fusion protein of claim 42, wherein the linker (L₁) is conjugated to C-terminus of the Fc domain monomer (B) and to the C-terminus of the polypeptide therapeutic agent (P₁).

51. The fusion protein of any one of claims 42-44, 49, 50, wherein L₁ comprises a chemical linker that is covalently conjugated to each of B and P₁.

52. The fusion protein of any one of claims 42-44, 49, 50, wherein B and P₁ are expressed as separate polypeptide chains and are subsequently each covalently conjugated to L₁.

53. The fusion protein of claim 42, wherein n₁ is 1, n₂ is 1, and the fusion protein comprises the structure:
P₂-L₂-B-L₁-P₁.

54. The fusion protein of claim 53, wherein
the linker (L₂) is conjugated to the C-terminus of the polypeptide therapeutic agent (P₂) and to the N-terminus of the Fc domain monomer (B), and
the linker (L₁) is conjugated to the C-terminus of the Fc domain monomer (B) and to the N-terminus of the polypeptide therapeutic agent (P₁).

55. The fusion protein of claim 53 or 54, wherein L₁ and L₂ are each an independently selected peptide linker comprising between 2 and 200 amino acids.

56. The fusion protein of claim 55, wherein L₁ and L₂ are each an independently selected peptide linker comprising between 5 and 25 amino acids.

57. The fusion protein of any one of claims 54-56, wherein L₁ and L₂ are each an independently selected peptide linker comprising the amino acid sequence of any one of (GS)ₓ, (GGS)ₓ, (GGGGS)ₓ, (GGSG)ₓ, (SGGG)ₓ, wherein x is an integer from 1 to 10.

58. The fusion protein of any one of claims 54-57, wherein P₂, L₂, B, L₁, and P₁ are expressed together as a single polypeptide chain.

59. The fusion protein of claim 54, wherein
the linker (L₂) is conjugated to the N-terminus of the polypeptide therapeutic agent (P₂) and to the N-terminus of the Fc domain monomer (B), and
the linker (L₁) is conjugated to the N-terminus of the polypeptide therapeutic agent (P₁) and to the C-terminus of the Fc domain monomer (B).

60. The fusion protein of claim 54, wherein
the linker (L₂) is conjugated to the C-terminus of the polypeptide therapeutic agent (P₂) and to the N-terminus of the Fc domain monomer (B), and
the linker (L₁) is conjugated to the C-terminus of the polypeptide therapeutic agent (P₁) and to the C-terminus of the Fc domain monomer (B).

61. The fusion protein of any one of claims 54, 55, 59, or 60, wherein L₂ comprises a chemical linker that is covalently conjugated to each of B and P₂, and L₁ comprises a chemical linker that is covalently conjugated to each of B and P₁.

62. The fusion protein of any one of claims 54, 55, 59, or 60, wherein P₂, B, and P₁ are expressed as separate polypeptide chains, P₂ and B are subsequently each covalently conjugated to L₂, and P₁ and B are subsequently each covalently conjugated to L₁.

63. The variant Fc domain monomer of any one of claims 1-33, the conjugate of any one of claims 34-39, or the fusion protein of any one of claims 40-62, wherein the Fc domain monomer dimerizes to form an Fc domain.

64. A pharmaceutical composition comprising a variant Fc domain monomer of any one of claims 1-33, a conjugate of any one of claims 34-39, a fusion protein of any one of claims 40-62, or the Fc domain of claim 63, and a pharmaceutically acceptable carrier.

65. A method of treating or preventing a respiratory disorder in a subject, the method comprising administering to the subject the pharmaceutical composition of claim 64.

66. The method of claim 65, wherein the respiratory disorder is an infection.

67. The method of claim 66, wherein the infection is a viral infection.

68. The method of claim 67, wherein the viral infection is selected from the group comprising RSV, Influenza, Dengue, a beta coronavirus, and Zika virus.

69. The method of claim 66, wherein the infection is a bacterial infection.

70. The method of claim 65, wherein the respiratory disorder is selected from the group comprising chronic obstructive pulmonary disease (COPD), chronic bronchitis, cystic fibrosis, bronchiectasis, and pneumonia.

71. The method of any one of claims 65-70, wherein a ratio of the concentration of the polypeptide, the conjugate, or the fusion protein in epithelial lining fluid is at least 30% of the concentration of the polypeptide, the conjugate, or the fusion protein in plasma within 2 hours after administration.

72. The method of claim 71, wherein the ratio of the concentration is at least 45% within 2 hours after administration.

73. The method of claim 71 or 72, wherein the ratio of concentration is at least 55% within 2 hours after administration.

74. The method of any one of claims 71-73, wherein the ratio of concentration is at least 60% within 2 hours after administration.

75. A method of treating or preventing a hepatic disorder in a subject, the method comprising administering to the subject the pharmaceutical composition of claim 64.

76. The method of claim 75, wherein the hepatic disorder is an infection.

77. The method of claim 76, wherein the infection is a viral infection.

78. The method of claim 76, wherein the viral infection is selected from the group comprising Hepatitis A, Hepatitis B, and Hepatitis C.

79. The method of claim 75, wherein the hepatic disorder is selected from the group comprising primary biliary cholangitis, primary sclerosing cholangitis, hepatocellular carcinoma, bile duct cancer, liver cell adenoma, nonalcoholic fatty liver disease (NAFLD), acute liver failure, and cirrhosis.

80. A method of treating or preventing a central nervous system (CNS) disorder in a subject, the method comprising administering to the subject the pharmaceutical composition of claim 64.

81. The method of claim 80, wherein the CNS disorder is an infection.

82. The method of claim 81, wherein the infection is a viral infection.

83. The method of claim 82, wherein the viral infection is selected from the group comprising viral meningitis, herpes simplex virus (HSV) 1, HSV 2, Epstein-Barr virus, varicella-zoster virus, poliovirus, coxsackievirus, West Nile virus, Lacrosse virus, western equine encephalitis, eastern equine encephalitis, Powassan virus, or rabies virus.

84. The method of claim 80, wherein the CNS disorder is selected from the group comprising cancer, Alzheimer disease, Parkinson disease, epilepsy, multiple sclerosis, schizophrenia, and meningitis.

85. A method of treating or preventing a muscle disorder in a subject, the method comprising administering to the subject the pharmaceutical composition of claim 64.

86. The method of claim 85, wherein the muscle disorder is cancer or myositis.

87. The method of claim 86, wherein the myositis is caused by an injury, an infection, or an immune disorder.

88. A method of treating or preventing a skin disorder in a subject, the method comprising administering to the subject the pharmaceutical composition of claim 64.

89. The method of claim 88, wherein the skin disorder is selected from the group comprising eczema, psoriasis, acne, rosacea, cold sores, cellulitis, basal cell carcinoma, squamous cell carcinoma, and melanoma.

90. A method of treating or preventing an ocular disorder in a subject, the method comprising administering to the subject the pharmaceutical composition of claim 64.

91. The method of claim 90, wherein the ocular disorder is selected from age-related macular degeneration, cataract, and glaucoma.

92. A method of treating or preventing a vascular disorder in a subject, the method comprising administering to the subject the pharmaceutical composition of claim 64.

93. A method of treating or preventing an infection in a subject, the method comprising administering to the subject the pharmaceutical composition of claim 64.

94. The method of claim 93, wherein the infection is a viral infection, a bacterial infection, or a fungal infection.
